SEQUENCE LISTING (2 sequences)

SEQ ID NO: 1 - protein, 51 aa, Artificial Sequence
  Adrenomedullin 2 - 52
  DISULFID (15)..(20)
  MOD_RES (51)..(51): AMIDATION
RQSMNNFQGLRSFGCRFGTCTVQKLAHQIYQFTDKDKDNVAPRSKISPQGY

SEQ ID NO: 2 - protein, 52 aa, Artificial Sequence
  modified Adrenomeddulin 1 - 52
  MOD_RES (1)..(1): modified Tyrosine residue
  DISULFID (16)..(21)
  MOD_RES (52)..(52): AMIDATION
YRQSMNNFQGLRSFGCRFGTCTVQKLAHQIYQFTDKDKDNVAPRSKISPQGY

(19) United States
(12) Patent Application Publication
(10) Pub. No.: US 2023/0364245 A1
(43) Pub. Date: Nov. 16, 2023

(54) PHARMACEUTICAL FORMULATIONS POLYETHYLENE GLYCOL-BASED PRODRUGS OF ADRENOMEDULLIN AND USE

(71) Applicant: Bayer Aktiengesellschaft, Leverkusen (DE)

(72) Inventors: Florian UNGER, Haan (DE); Stefan Christian SCHNEID, Duesseldorf (DE); Hans-Walter MOTZKUS, Schildow (DE); Carina HAASBACH, Wuppertal (DE)

(21) Appl. No.: 17/905,747

(22) PCT Filed: Mar. 31, 2021
(86) PCT No.: PCT/EP2021/058428

§ 371 Date: Sep. 6, 2022

(30) Foreign Application Priority Data
Apr. 3, 2020 (EP) 20168075.8

(51) Int. Cl. A61K 47/60; A61K 47/26; A61K 9/00

(52) U.S. Cl. CPC A61K 47/60; A61K 9/0078; A61K 47/26

(57) ABSTRACT

The present invention relates to novel pharmaceutical formulations, preferably for inhalation, comprising polyethylene glycol (PEG)-based prodrugs of Adrenomedullin (PEG-ADM) and the use thereof for the treatment and/or prevention of acute lung injury/acute respiratory distress syndrome (ALI/ARDS).

The present invention relates to novel pharmaceutical formulations for inhalation comprising polyethylene glycol (PEG)-based prodrugs of Adrenomedullin (ADM) and the use thereof for the treatment and/or prevention of acute lung injury/acute respiratory distress syndrome (ALI/ARDS).

The 52 amino acid peptide hormone ADM is produced in adrenal gland, lung, kidney, heart muscle and other organs. The plasma levels of ADM are in the lower picomolar range. ADM is a member of the calcitonin gene-related peptide (CGRP) family of peptides and as such binds to a heterodimeric G-protein coupled receptor that consists of CRLR and RAMP 2 or 3 (Calcitonin-receptor-like receptor and receptor activity modifying protein 2 or 3). Activation of the ADM receptor leads to intracellular elevation of adenosine 3′, 5′-cyclic monophosphate (cAMP) in the receptor-bearing cells. ADM receptors are present on different cell types in almost all organs including endothelial cells. ADM is thought to be metabolized by neutral endopeptidase and is predominantly cleared in the lung where ADM-receptors are highly expressed [Gibbons C., et al., Mol Endocrinol 21(4), 783-796 (2007)].

Experimental data from the literature suggest that ADM is involved in a variety of functional roles that include, among others, blood pressure regulation, bronchodilatation, renal function, hormone secretion, cell growth, differentiation, neurotransmission, and modulation of the immune response. Moreover, ADM plays a crucial role as autocrine factor during proliferation and regeneration of endothelial cells [Garcia M.A., et al., Expert Opin Ther Targets, 10(2), 303-317 (2006)].

There is an extensive body of evidence from the literature which shows that ADM is indispensable for an intact endothelial barrier function and that administration of ADM to supra-physiological levels exerts strong anti-edematous and anti-inflammatory functions in a variety of inflammatory conditions in animal experiments including sepsis, acute lung injury and inflammation of the intestine [Temmesfeld-Wollbrück B., et al., Thromb Haemost; 98, 944-951 (2007)].

Clinical testing of ADM was so far conducted in cardiovascular indications with a measurable hemodynamic end point such as pulmonary hypertension, hypertension, heart failure and acute myocardial infarction. ADM showed hemodynamic effects in several studies in patients suffering from the aforementioned conditions. However, effects were only short lasting and immediately ceasing after the end of administration. These findings correlated well with the known pharmacokinetic profile of ADM. Pharmacodynamic effects comprised among others lowering of systemic and pulmonary arterial blood pressure and increase of cardiac output [Troughton R.W., et al., Hypertension, 36(4), 588-93 (2000); Nagaya N. and Kangawa K., Peptides, 25(11), 2013-8 (2004); Kataoka Y., et al., J Cardiovasc Pharmacol, 56(4), 413-9 (2010)].

In this respect, compounds described in WO 2013/064508 A1 (“PEG-ADM”) act as slow release prodrugs of ADM with extended duration of pharmacological action as compared to “free” ADM and on the basis of this specific action mechanism exert in vivo sustained anti-inflammatory and hemodynamic effects such as stabilization of endothelial barrier function, and reduction of blood pressure, respectively.

The compounds according to WO 2013/064508 A1 can act systemically and/or locally. For this purpose, they can be administered in a suitable way, for example as a pharmaceutical aerosol intended for inhalation by means of a suitable inhaler device.

The respiratory tract is directly accessible from the outside and thus, an attractive avenue for a targeted administration of therapeutic agents. The basic concept of inhalation is utilized for the treatment of numerous respiratory diseases, owing to the advantages of this approach such as a rapid onset of drug action, high local drug concentration, superior therapeutic selectivity and reduction of side effects [Rau J.L., Respir Care, 50(3), 367-82 (2005)]. The lungs can be accessed by inhalation deposition of different types of pharmaceutical aerosols. Typically, these formulations are composed of particles or droplets (together referred to as “particles” throughout this specification) of a few microns in diameter containing the active ingredient (Hofmann W., J Aerosol Sci, 42(10), 693-724 (2011). Having the significant impact of aerosols’ physicochemical properties for lung deposition and hence, therapeutic efficacy of the delivered medication in mind, formulation and device design development are currently aiming for a production of optimized aerosols [Dolovich M. and Dhand R., Lancet 377(9770), 1032-45 (2011)].

PEG-ADM is under development as a therapeutic agent for inhalation. The stability of PEG-ADM in solution in liquid state is insufficient for long-term storage as the molecule can be degraded through different pathways such as aggregation, linker separation or disulphide oxidation. In addition, even if a reasonably stable formulation is found, it should be noticed that it must also be suitable for nebulization. Furthermore, it is also of importance that a therapeutically effective concentration is finally delivered to the patient.

Lyophilization is a complex process that requires a careful balancing of product, equipment, and processing techniques (see International Journal of Novel Trends in Pharmaceutical Sciences. 3(4). 2013). Besides various advantages, lyophilization also bears many disadvantages such as long processing times, aseptic processing, limitations regarding size and filling volume of suitable containers and the related costs (Pikal MJ 2002. Freeze Drying. Encyclopedia of Pharmaceutical Technology: 1299-1326). As lyophilization is a complex process, a certain experience and knowledge about critical formulation temperature/collapse temperature of the formulation and freeze-drying parameters is needed (Carpenter JF, Chang BS, Garzon-Rodriguez W, Randolph TW 2002. Rational design of stable lyophilized protein formulations: theory and practice. Pharm Biotechnol 13:109-133). Cake structure of the lyophilizate and solid state of active and inactive ingredients is affected by both composition and processing parameters.

As is known in the field of pharmaceutical formulation, lyophilization, also known as freeze drying, is a method of processing a liquid product into a dry solid product. In general, lyophilization is defined as a stabilizing process in which the product is frozen followed by elimination of the water content by sublimation. The resulting lyophilized product should have an acceptable cake structure and sufficient stability (“shelf-life”), short rehydration/reconstitution time, and sufficient in-use- stability at the required temperature (Wang W 2000. Lyophilization and development of solid protein pharmaceuticals. Int J Pharm 203(1-2):1-60).

For the development of a stable lyophilizate, both the combination of excipients, their respective concentration in the solution prior to freeze drying, and the lyophilization process parameters are of critical importance. The process design needs to consider the propensity of solutes to crystallize during the freezing or thermal treatment process, account for the product temperature not to surpass the critical formulation during primary drying, to compensate increases in the product temperature based on the evolution of product resistance, and the secondary drying conditions to obtain a suitable residual moisture content compatible with storage over an extended time (Costantino HR, Pikal MJ, American Association of Pharmaceutical Scientists. 2004. Lyophilization of biopharmaceuticals. Arlington, VA: AAPS Press).

Subsequent to reconstitution, the resulting solution needs to be compatible with the intended application mode. This can be challenging e.g. in case of high viscosity of the reconstituted solution, especially if the solution needs to be injected through a narrow canula or if the solution is nebulized for inhalation application. Especially for vibrating mesh nebulizers, an influence of changes in viscosity on the nebulization properties have been reported (Chan JGY, Traini D, Chan HK, Young PM, Kwok PCL. Delivery of High Solubility Polyols by Vibrating Mesh Nebulizer to Enhance Mucociliary Clearance. J Aerosol Medicine and Pulmonary Drug Delivery 2012, 297-305). In addition, the integrity of the therapeutic molecule needs to be preserved during the application step.

An object of the present invention is to provide a stable pharmaceutical formulation comprising PEG-based prodrugs of ADM (PEG-ADM), which are delivered to the respiratory tract via inhalation.

Another object of the present invention is to provide suitable stable pharmaceutical formulations comprising PEG-based prodrugs of ADM (PEG-ADM) for treatment and/or prevention of ALI/ARDS, which are delivered to the respiratory tract via inhalation.

Moreover, it was an object of the present invention to allow the nebulization of formulations of PEG-ADM of therapeutically-relevant concentrations. The object of the present invention is to provide suitable pharmaceutical formulations comprising PEG-based prodrugs of ADM (PEG-ADM) for treatment and/or prevention of ALI/ARDS, which are delivered to the respiratory tract via inhalation. Moreover, it was an object of the present invention to allow the nebulization of formulations of PEG-ADM of therapeutically-relevant concentrations by means of vibrating-mesh nebulizers.

Vibrating-mesh nebulizers are generally described in for example US 6,467,476 B1, US 8,398,001 B2 or US 7,331,339 B2. Vibrating-mesh nebulizers comprise a thin plate, usually made from metal, the so-called mesh. The mesh comprises a front surface and a rear surface. The mesh has a plurality of apertures extending between the front surface and the rear surface. In some embodiments the apertures are tapered to narrow from the rear surface to the front surface. The liquid to be nebulized is usually in a reservoir in fluid communication with the rear surface of the mesh.

The efficiency of formulation nebulization (i.e. size of the generated aerosol particles and the output rate, whereby the output rate is defined as the mass of aerosol delivered by the nebulizer device per time) is on one hand a function of the aperture cross-section of the vibratory mesh of the employed vibrating-mesh nebulizer. On the other hand, the physicochemical properties of the utilized formulation also reveal significant impact on the delivery of aerosol particles from the nebulizer device. A number of studies investigated the interplay of formulation parameters with the mode of vibrating-mesh nebulization [Beck-Broichsitter M. and Oesterheld N., Eur J Pharm Biopharm, 119, 11-6 (2017)] in order to match the performance to the requirements of the individual application.

Micron-scale aperture dimensions are required for the generation of fine medicament mists suitable for inhalation to the deep lungs. However, the fabrication of apertures suitable for generating smallest particles is challenging [Kohno M. and Matsuoka Y., JSME Int J, Ser B 47(3), 497-500 (2004); Shen et al., Sens. Actuators A, 144(1), 135-43 (2008)]. Furthermore, despite sophisticated techniques being around to fabricate the aperture diameter of meshes to dimensions smaller than 5 µm, the variations in size between the apertures in a single mesh are still considerable due to the small overall dimensions. This will directly lead to significant differences of the efficiency of formulation nebulization from one vibrating-mesh nebulizer to another for the same pharmaceutical formulation.

One such example are aqueous formulations of PEG-ADM (i.e. a 40 kDa PEG conjugated to ADM; cf. compound according to formula (Ia) below) when nebulized by means of the Aerogen® Solo. PEG-ADM (see WO 2013/064508 A1) is described as compound which act as slow release ADM-prodrug with extended duration of pharmacological action which is intended for an application to self-breathing and ventilated patients. The Aerogen® Solo device is well-known to the person skilled in the art [El Hansy M., et al., Pulm Pharmacol Ther, 45(XX), 159-63 (2017); Dugernier J., et al., Ann Intensive Care, 6, 73 (2016); Ari A., et al., Respir Care 55(7), 837-44 (2010)].

Surprisingly, it has been shown that the pharmaceutical formulation according to the invention has the following surprising technical effects
the pharmaceutical formulation is stable;
the pharmaceutical formulation can be provided as sterile dosage form;
the pharmaceutical formulation can be easily provided as lyophilizate, reconstituted and nebulized;
the pharmaceutical formulation can be easily provided as lyophilizate, reconstituted and nebulized at least three times without relevant changes to the nebulization performance for the nebulizer used;
the pharmaceutical formulation can be provided as sterile dosage form with improved stability at higher temperature
the pharmaceutical formulation is stable and shows good nebulization properties;
the pharmaceutical formulation is stable, even after lyophilization and reconstitution in a solvent;
the pharmaceutical formulation shows after freeze-drying and reconstitution still good stability and nebulization properties; and
the lyophilizate and/or reconstituted lyophilizate according to the invention shows a constant droplet size after multiple nebulization. This is beneficial as the same nebulizer can be used several times.

The invention provides
(1) A pharmaceutical formulation comprising PEG-ADM. A pH regulator and trehalose;
(2) A lyophilizate comprising PEG-ADM. A pH regulator and trehalose;
(3) A liquid pharmaceutical formulation comprising PEG-ADM, a pH regulator, trehalose and optionally an osmolarity regulator, wherein the pharmaceutical formulation has a pH between 3 to 5 and an osmolar concentration of 150 mosmol/l to 450 mosmol/L;
(4) A pharmaceutical formulation comprising a lyophilizate according to (2) and a solvent (reconstituted lyophilizate);
(5) A lyophilizate obtainable by freeze-drying of the liquid pharmaceutical formulation according to (3);
(6) A lyophilizate according to (2) obtainable by freeze-drying of the liquid pharmaceutical formulation according to (3);
(7) A liquid pharmaceutical formulation according to (3) or (5) obtainable by mixing the lyophilizate according to (2) or (4) with a solvent;
(8) The pharmaceutical formulation according to any one of (1) to (7) for inhalation;
(9) A medicament comprising the pharmaceutical formulation according to any one of (1) to (7);
(10) A combined pharmaceutical dose comprising the pharmaceutical formulation according to any one of (1) to (7);
(11) Combination pack comprising the pharmaceutical formulation according to any one of (1) to (7);
(12) The pharmaceutical formulation according to any one of (1) to (11) for use in the treatment and/or prevention of diseases;
(13) The use of the pharmaceutical formulation according to any one of (1) to (12) for the treatment and/or prevention of a disease and/or disorder;
(14) The pharmaceutical formulation according to any one of (1) to (12) for producing a medicament for treatment and/or prevention of a disease and/or disorder;
(15) A method of treatment and/or prevention of a disorder and/or disease comprising administering the pharmaceutical formulation according to any one of (1) to (12); and
(16) A method for preparing the pharmaceutical formulation according to any one of (1) to (12).

### I. Pharmaceutical Formulations

The disclosure in this section, i.e. in
section I.i. PEG-ADM (component a);
section I.ii. Solvent (component b);
section I.iii. PH-regulator (component c);
section I.ix. Osmolarity regulator (component d); and/or
section I.v. Trehalose (component e)
refers to embodiments of components a to e and can be applied to any one of the embodiments of the invention disclosed in section II. (pharmaceutical formulation), and/or section III (liquid pharmaceutical formulation) and/or section IV (reconstituted lyophilizate) below.

The disclosure in
section V. Excipients;
section VI. Combined pharmaceutical dosage form;
section VII. Combination pack;
section VIII. Indications; and/or
section IX. Product-by-process
can be applied to any one of the embodiments of the invention disclosed in section II. (pharmaceutical formulation), section III (liquid pharmaceutical formulation) and/or section IV (reconstituted lyophilizate) below.

The concentrations given in “wt.-%” (“percentage per mass” or “weight percentage”) of components a, c, d, e are based on the total dry weight of the pharmaceutical formulation. It is the mass fraction of a substance within a mixture is the ratio wᵢ of the mass mᵢ of that substance to the total mass mₜₒₜ of the mixture. Expressed as a formula, the mass fraction is:

$${w}_{i} = \frac{{m}_{i}}{{m}_{t o t}} .$$

Because the individual masses of the ingredients of a mixture sum mₜₒₜ, their mass fractions sum to unity:

$${\sum }_{i = 1}^{n} {w}_{i} = 1.$$

Mass fraction can also be expressed, with a denominator of 100, as percentage by mass (in commercial contexts often called percentage by weight, abbreviated wt.-%). It is one way of expressing the composition of a mixture in a dimensionless size.

The concentrations given in “mg/ml” (“milligram per milliliter”) of components a, c, d, e are based on the total volume of the liquid pharmaceutical formulation.

### I. PEG-ADM (component A)

The pharmaceutical formulation according to the invention comprises PEG-ADM. The term “the compound of formula (I)” or “compound according to the general formula (I)” or “PEG-ADM” or “PEG-based prodrugs of ADM” or “component a” are used as synonyms and refer to a compound according to the general formula (I),

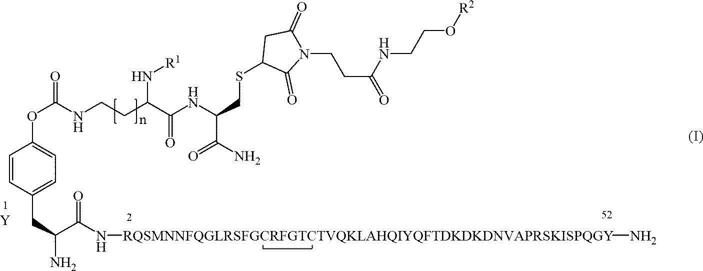

in which
n represents the number 0, 1, 2 or 3,
R¹ represents hydrogen, methyl, ethyl, n-propyl or isopropyl,
R² represents linear or branched PEG 20 kDa to 80 kDa endcapped with a methoxy-group.

The term “PEG-ADM” also comprises a hydrate thereof, solvate thereof, salt thereof, pharmaceutically acceptable salt thereof, or the solvates of salts thereof. Thus, “PEG-ADM” is a synonym for the compounds according to formula (I), compounds according to formula (Ia), a hydrate thereof, solvate thereof, salt thereof, pharmaceutically acceptable salt thereof, or the solvates of salts thereof. The synthesis of PEG-ADM is described in WO 2013/064508 A1. PEG-ADM acts as a prodrug. In the body, adrenomedullin (ADM) is released from PEG-ADM. This is described in detail in WO 2013/064508 A1. In one embodiment the pharmaceutical formulation the PEG-ADM is selected from compounds of the general formula (I),

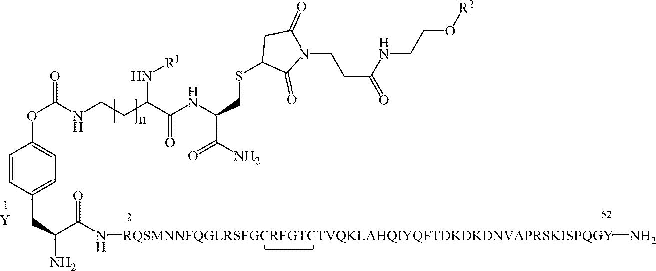

(I), in which
n represents the number 0, 1, 2 or 3,
R¹ represents hydrogen, methyl, ethyl, n-propyl or isopropyl,
R² represents linear or branched PEG 20 kDa to 80 kDa endcapped with a methoxy-group,
a hydrate thereof, solvate thereof, salt thereof, pharmaceutically acceptable salt thereof, or the solvates of salts thereof.

In one embodiment the pharmaceutical formulation the PEG-ADM is selected from compounds of the formula (I) in which
n represents the number 1 or 2,
R¹ represents hydrogen or methyl,
R² represents linear PEG 40 kDa endcapped with a methoxy-group.

In one embodiment the pharmaceutical formulation the PEG-ADM is selected from compounds of the formula (I),

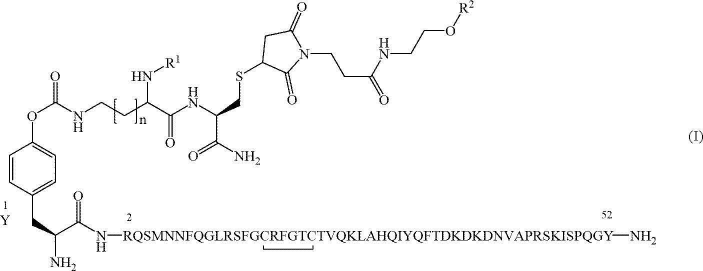

in which
n represents the number 1 or 2,
R¹ represents hydrogen,
R² represents linear PEG 40 kDa endcapped with a methoxy-group.

In one embodiment the pharmaceutical formulation the PEG-ADM is the compound according to formula (Ia)

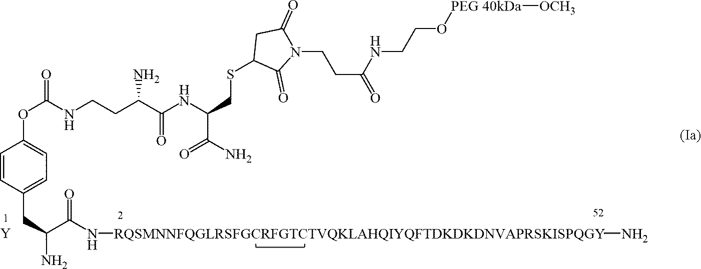

The compound according to formula (Ia) is described in detail in WO 2013/064508 A1. Its CAS number is 1432735-93-7.

In one embodiment of the pharmaceutical formulation according to the invention the PEG-ADM is the compound according to formula (Ia), a hydrate thereof, solvate thereof, salt thereof, pharmaceutically acceptable salt thereof, or the solvates of salts thereof.

Depending on their structure, the compounds according to the invention may exist in stereoisomeric forms (enantiomers, diastereomers). The invention therefore embraces the enantiomers or diastereomers and the particular mixtures thereof. The stereoisomerically homogeneous constituents can be isolated in a known manner from such mixtures of enantiomers and/or diastereomers.

When the compounds according to the invention can occur in tautomeric forms, the present invention embraces all tautomeric forms.

In the context of the present invention, preferred salts are physiologically acceptable salts of the compounds according to the invention.

“Physiologically acceptable salts” or “pharmaceutically acceptable salts” of the compounds according to the invention include acid addition salts of mineral acids, carboxylic acids and sulfonic acids, for example salts of hydrochloric acid, hydrobromic acid, sulfuric acid, phosphoric acid, methane sulfonic acid, ethane sulfonic acid, toluene sulfonic acid, benzenesulfonic acid, naphthalene disulfonic acid, acetic acid, trifluoroacetic acid, propionic acid, lactic acid, tartaric acid, maleic acid, citric acid, fumaric acid, maleic acid and benzoic acid.

“Physiologically acceptable salts” or “pharmaceutically acceptable salts” of the compounds according to the invention also include salts of customary bases, for example and with preference alkali metal salts (e.g. sodium and potassium salts), alkaline earth metal salts (e.g. calcium and magnesium salts) and ammonium salts derived from ammonia or organic amines having 1 to 16 carbon atoms, for example and with preference ethylamine, diethylamine, triethylamine, ethyl-diiso-propyl-amine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine. Suitable pharmaceutically acceptable salts that can be used in the combination according to the invention are well known to those skilled in the art and include salts of inorganic acids, organic acids, inorganic bases, alkaline cations, alkaline earth cations and organic bases. In one embodiment the pharmaceutically acceptable salt can be selected from hydrochloric acid, hydrobromic acid, sulfuric acid, phosphoric acid, methane sulphonic acid, trifluoromethanesulfonic acid, benzenesulfonic acid, p-toluene sulfonic acid, 1-naphthalenesulfonic acid, 2-naphthalenesulfonic acid, acetic acid, trifluoroacetic acid, malic acid, tartaric acid, citric acid, lactic acid, oxalic acid, succinic acid, fumaric acid, maleic acid, benzoic acid, salicylic acid, phenylacetic acid, and mandelic acid acetate, benzoate, besylate, bromide, camsylate, carbonate, citrate, edisylate, estolate, fumarate, gluceptate, gluconate, glucuronate, hippurate, iodide, isethionate, lactate, lactobionate, malate, maleate, mesylate, methylsulfate, napsylate, nitrate, oxalate, pamoate, phosphate, stearate, succinate, sulfate, tartrate, bitartrate, tosylate, calcium, diolamine, lithium, lysine, magnesium, meglumine, N-methylglucamine, olamine, potassium, tromethamine, tris(hydroxymethyl)aminomethane, benzenesulfonate, ethanesulfonate and zinc.

In one embodiment the pharmaceutically acceptable salt can be selected from hydrochloride, sulfate, mesylate, tosylate, tartrate, citrate, benzenesulfonate, ethanesulfonate, maleate, and phosphate

In the context of the invention, solvates refer to those forms of the compounds according to the invention which, in the solid or liquid state, form a complex by coordination with solvent molecules. Hydrates are a specific form of the solvates, in which the coordination is with water. Preferred solvates in the context of the present invention are hydrates.

### ii. Solvent (component b)

The pharmaceutical formulation according to the invention comprises a solvent. The term “solvent” is used as typically in the art. The terms “solvent” and “component b” are synonyms. The term solvent refers to pure solvents and/or to mixtures of different solvents.

In one embodiment of the pharmaceutical formulation according to the invention, the solvent is selected from the group of water, sodium chloride solution, citric acid, hydrochloric acid, sodium hydroxide solution, sodium citrate solution and/or mixtures thereof.

The term “citric acid” used herein, also encompasses any salt, pharmaceutical acceptable salt, derivative or mixture thereof. Further examples of salt, pharmaceutical acceptable salt, derivative of citric acid encompass citric acid anhydrous, sodium citrate and citric acid monohydrate.

In one embodiment of the pharmaceutical formulation according to the invention, the solvent comprises water. In one embodiment of the pharmaceutical formulation according to the invention, the solvent consists of water.

In one embodiment of the pharmaceutical formulation according to the invention, the solvent comprises a mixture of water and sodium chloride. In one embodiment of the pharmaceutical formulation according to the invention, the solvent consists of a mixture of water and sodium chloride.

In one embodiment of the pharmaceutical formulation according to the invention, the solvent comprises a mixture of water and citric acid. In an alternative of this embodiment the citric acid can be selected from citric acid, sodium citrate and citric acid monohydrate. In one embodiment of the pharmaceutical formulation according to the invention, the solvent consists of a mixture of water and citric acid. In an alternative of this embodiment the citric acid can be selected from citric acid, sodium citrate and citric acid monohydrate.

In one embodiment of the pharmaceutical formulation according to the invention, the solvent comprises a mixture of water and sodium citrate. In one embodiment of the pharmaceutical formulation according to the invention, the solvent consists of a mixture of water and sodium citrate.

In one embodiment of the pharmaceutical formulation according to the invention, the solvent comprises a mixture of water and hydrochloric acid. In one embodiment of the pharmaceutical formulation according to the invention, the solvent consists of a mixture of water and hydrochloric acid.

In one embodiment of the pharmaceutical formulation according to the invention, the solvent comprises a mixture of water and sodium hydroxide. In one embodiment of the pharmaceutical formulation according to the invention, the solvent consists of a mixture of water and sodium hydroxide.

In one embodiment, the solvent comprises a buffer. Examples of buffers that can be used in this embodiment are described in detail in section “pH regulator” below. In one embodiment the solvent consists of a buffer. Examples of buffers that can be used in this embodiment are described in detail in section “pH regulator” below.

In one embodiment the solvent is a reconstitution medium. A “reconstitution medium” is a solvent used for solving, dissolving, diluting or dispersing the pharmaceutical formulation, the liquid pharmaceutical formulation and/or a lyophilizate of the aforementioned formulations. In one embodiment a lyophilizate according to any one of the embodiments disclosed herein is solved, dissolved or dispersed by mixing said lyophilizate with the solvent. Here, said lyophilizate is “reconstituted” in the solvent.

In one embodiment, the solvent is or comprises water. In one embodiment, the solvent is a sodium chloride solution. Examples of sodium chloride solutions are hypotonic, isotonic and hypertonic sodium solutions. For example, a 0.9% solution of NaCl (0.9 gram in 100 mL water) is isotonic. A “hypotonic solution” has a concentration of less than 0.9%. A “hypertonic solution” has a concentration of more than 0.9%. Sometimes “sodium chloride solution” is used synonymously with “saline solution”.

### iii. pH-regulator (component c)

The pharmaceutical composition according to the invention comprises a pH regulator. The term “pH regulator” and “component c” are synonyms. The term “pH regulator” comprises substances that regulate the pH. The term “pH regulator” also refers to a plurality of pH regulators. The term “pH regulator” refers to one pH regulator or two or more pH regulators. Thus, the term “pH regulator” also encompasses mixtures comprising or consisting of different pH regulators.

One example of a pH regulator is a buffer system. A “buffer” consists of a mixture of a weak acid and its conjugate base, or vice versa. Its pH changes very little when a small amount of strong acid or base is added to it. Buffer solutions are used as a means of keeping pH at a nearly constant value in a wide variety of chemical applications. One example is the system citrate / citric acid. The citrate is the salt of citric acid, e.g. the sodium salt, the potassium salt or the calcium salt of citric acid. Further examples of salts, pharmaceutical acceptable salts, derivatives of citric acid encompass citric acid anhydrous, sodium citrate and citric acid monohydrate. Embodiments of buffers that can be used in the formulations according to the invention are citrate buffer (pH 3-6.2; pKa 3.3/4.8/6.4), phosphate citrate buffer (pH 2.2-8.0, pKa = 7.2/6.4/2.2), phosphate buffer (pH 2-12; pKa 2.2/6.9/12.3), sodium acetate buffer (pH 3.6-5.6, pKa 4.76), glycine-HCl (pH 2.2-3.6, pKa 2.35), leucine buffer (pH 2-4; pKa 2.3), aspartic acid buffer (pH 3-5; pKa 2.0/3.9), glutamic acid buffer (pH 3-6; pKa 2.2/4.3). Even if not explicitly stated herein, any buffer that is suitable for adjusting the pH to 3 to 5 can be used in the pharmaceutical formulation according to the invention.

In one embodiment the pH regulator comprises citric acid, a salt of citric acid, a pharmaceutical acceptable salt of citric acid, a derivative of citric acid, and/or mixtures thereof.

In one embodiment the pH regulator comprises hydrochloric acid, citric acid, a salt of citric acid, pharmaceutical acceptable salt of citric acid, derivative of citric acid, and/or mixtures thereof.

In one embodiment, the pH regulator comprises hydrochloric acid.

In one embodiment, the pH regulator comprises a mixture comprising hydrochloric acid and sodium hydroxide. In one embodiment, the pH regulator comprises a mixture comprising hydrochloric acid, sodium hydroxide and citric acid. In one embodiment, the pH regulator comprises a mixture comprising sodium hydroxide and citric acid. In one embodiment, the pH regulator comprises a mixture comprising sodium citrate and hydrochloric acid. In an alternative of these embodiments listed before, the citric acid is a salt of citric acid, pharmaceutical acceptable salt of citric acid, a derivative of citric acid and/or mixtures thereof, preferably citric acid anhydrous, sodium citrate and citric acid monohydrate.

In one embodiment, the pH regulator consists of hydrochloric acid. In one embodiment, the pH regulator consists of a mixture comprising hydrochloric acid and sodium hydroxide. In one embodiment, the pH regulator consists of a mixture comprising hydrochloric acid, sodium hydroxide and citric acid. In one embodiment, the pH regulator consists of a mixture comprising sodium hydroxide and citric acid. In one embodiment, the pH regulator consists of a mixture comprising sodium citrate and hydrochloric acid. In an alternative of the embodiments listed before, the citric acid is a salt of citric acid, pharmaceutical acceptable salt of citric acid, a derivative of citric acid and/or mixtures thereof, preferably citric acid anhydrous, sodium citrate and citric acid monohydrate.

In one embodiment the pharmaceutical composition according to the invention comprises at least one pH regulator. In one embodiment the pharmaceutical composition according to the invention comprises two or more pH regulators. In one embodiment the pharmaceutical composition according to the invention comprises three or more pH regulators. In one embodiment the pharmaceutical composition according to the invention comprises mixtures of pH regulators.

When a plurality of pH regulators is given, the sum of the concentrations of these pH regulators are the total concentration of the pH regulator. For example, if a concentration of 1 mg/ml citric acid und 1 mg/ml sodium hydroxide is given, the total concentration is 2 mg/mL pH regulator. For example, if a concentration of 1 wt.-% citric acid und 1 wt.-% sodium hydroxide is given, the total concentration is 2 wt.-% pH regulator.

### iv. Osmolarity regulator (component d)

The pharmaceutical formulation according to the invention comprises an osmolarity regulator. The term “osmolarity regulator” and “component d” are synonyms. The term “osmolarity regulator” refers to one osmolarity regulator as well as to mixtures of one two or more compounds for adjusting osmolarity. The osmotic concentration was determined via freezing-point depression [Osmomat 030, Gonotec, Model 030-D3P].

In one embodiment of the pharmaceutical composition according to the invention the osmolarity regulator is sodium chloride, citric acid, a salt, pharmaceutical acceptable salt, derivative of citric acid and/or mixtures thereof.

In one embodiment of the pharmaceutical composition according to the invention the osmolarity regulator is citric acid, a salt, pharmaceutical acceptable salt, derivative of citric acid. In one embodiment of the pharmaceutical composition according to the invention the osmolarity regulator is a salt, pharmaceutical acceptable salt, derivative of citric acid selected from the group consisting of citric acid anhydrous, sodium citrate and citric acid monohydrate.

In one embodiment of the pharmaceutical composition according to the invention the osmolarity regulator is sodium chloride.

When a plurality of osmolarity regulators is given, the sum of the concentrations of these osmolarity regulators are the total concentration of the pH regulator. For example, if a concentration of 1 mg/ml sodium chloride und 1 mg/ml citric is given, the total concentration is 2 mg/mL osmolarity regulators. For example, if a concentration of 1 wt.-% citric acid und 1 wt.-% sodium hydroxide is given, the total concentration is 2 wt.-% osmolarity regulators.

### v. Trehalose (component e)

The pharmaceutical formulation comprises trehalose. The term “trehalose” or “component e” are used as synonyms. “trehalose” also encompasses to any derivative thereof, solvates thereof, hydrates thereof and/or mixtures thereof.

In one embodiment trehalose is selected from the group of trehalose dihydrate, trehalose anhydrate and/or mixtures thereof.

### II. Pharmaceutical Formulation (wt.-%)

The invention provides for:

A pharmaceutical formulation comprising:
PEG-ADM (component a), wherein the PEG-ADM is a compound according to the general formula (I),

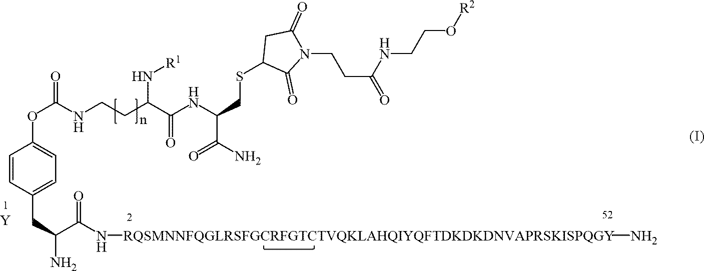

in which
n represents the number 0, 1, 2 or 3,
R¹ represents hydrogen, methyl, ethyl, n-propyl or isopropyl,
R² represents linear or branched PEG 20 kDa to 80 kDa endcapped with a methoxy-group,
or a hydrate thereof, solvate thereof, salt thereof, pharmaceutically acceptable salt thereof,
or the solvates of salts thereof;

a pH regulator (component c); and
trehalose or a hydrate thereof, solvate thereof, salt thereof, pharmaceutically acceptable salt thereof, or the solvates of salts thereof;
wherein the concentrations of components are based on the total weight of the pharmaceutical formulation.

In one embodiment the pharmaceutical formulation according to the invention is for inhalation and/or inhalative use. In one embodiment the pharmaceutical formulation is a lyophilizate.

The pharmaceutical formulation comprises at least PEG-ADM (component a), a pH regulator (component c), and trehalose (component e).

These components are described in section I (sections I.i to I.v.) detail above and display embodiments that can be used in the liquid pharmaceutical formulation described in this section II. The embodiments of the concentrations of the respective components comprised in the pharmaceutical formulation are described in section II (sections II.vi. to II.x) in detail below. A method for the preparation of the liquid pharmaceutical formulation is described in section II.x. below.

Accordingly, even if not stated specifically in the embodiments of the invention disclosed herein, the following features apply to all embodiments of the pharmaceutical formulation disclosed in this section III.:
when referring to “PEG-ADM” also a compound according to formula (I), a hydrate thereof, solvate thereof, salt thereof, pharmaceutically acceptable salt thereof, or the solvates of salts thereof, are meant; and
the concentrations of components a, c and d are based on the total weight of the pharmaceutical formulation.

The wt.-% are calcuted on the dry formulation (0 % residual moisture from water).

The concentrations given in “wt.-%” (“percentage per mass” or “weight percentage”) of components a, c, d, e are based on the total dry weight of the pharmaceutical formulation. It is the mass fraction of a substance within a mixture is the ratio wᵢ of the mass mᵢ of that substance to the total mass mₜₒₜ of the mixture. Expressed as a formula, the mass fraction is:

$${w}_{i} = \frac{{m}_{i}}{{m}_{\text{tot}}} .$$

Because the individual masses of the ingredients of a mixture sum mₜₒₜ, their mass fractions sum to unity:

$${\sum }_{i = 1}^{n} {w}_{i} = 1.$$

Mass fraction can also be expressed, with a denominator of 100, as percentage by mass (in commercial contexts often called percentage by weight, abbreviated wt.-%). It is one way of expressing the composition of a mixture in a dimensionless size.

The concentrations given in “mg/ml” (“milligram per milliliter”) of components a, c, d, e are based on the total volume of the liquid pharmaceutical formulation.

The statement regarding the overlapping functionalities of component c and d as described under section III equally apply here. The components c comprised in the pharmaceutical formulation can act also as osmolarity regulator (component d.). This means they can have overlapping functionality.

In one embodiment the PEG-ADM (component a) is selected from the embodiments disclosed under section I.i. above. In one embodiment the pH regulator (component c) is selected from the embodiments disclosed under section I.iii. above. In one embodiment the osmolarity regulator (component d) is selected from the embodiments disclosed under section I.iv. above. In one embodiment the trehalose (component e) is selected from the embodiments disclosed under section I.v. above.

In one embodiment the PEG-ADM (component a) is selected from the embodiments disclosed under section I.i. above, the pH regulator (component c) is selected from the embodiments disclosed under section I.iii. above, and/or the trehalose (component e) is selected from the embodiments disclosed under section I.v. above.

In one embodiment the PEG-ADM (component a) is selected from the embodiments disclosed under section I.i. above, the pH regulator (component c) is selected from the embodiments disclosed under section I.iii. above, the osmolarity regulator (component d) is selected from the embodiments disclosed under section I.iv. above, and/or the trehalose (component e) is selected from the embodiments disclosed under section I.v. above.

In one embodiment the PEG-ADM (component a) is selected from the embodiments disclosed under section I.i. above, the pH regulator (component c) is selected from the embodiments disclosed under section I.iii. above, and the trehalose (component e) is selected from the embodiments disclosed under section I.v. above.

In one embodiment the PEG-ADM (component a) is selected from the embodiments disclosed under section I.i. above, the pH regulator (component c) is selected from the embodiments disclosed under section I.iii. above, the osmolarity regulator (component d) is selected from the embodiments disclosed under section I.iv. above, and the trehalose (component e) is selected from the embodiments disclosed under section I.v. above.

### i. PEG-ADM (Component A) - Concentrations (wt.-%)

In one embodiment the PEG-ADM (component a) is selected from the embodiments disclosed under section I.i. above.

In one embodiment, the pharmaceutical formulation comprises 3 wt.-% to 10 wt.-% PEG-ADM, wherein the concentration is based on the total weight of the pharmaceutical formulation. In one alternative, the pharmaceutical formulation is a lyophilizate.

In one embodiment, the pharmaceutical formulation comprises 5 wt.-% to 8 wt.-% PEG-ADM, wherein the concentration is based on the total weight of the pharmaceutical formulation. In one alternative, the pharmaceutical formulation is a lyophilizate.

In one embodiment, the pharmaceutical formulation comprises 5.5 wt.-% to 7 wt.-% PEG-ADM, wherein the concentration is based on the total weight of the pharmaceutical formulation. In one alternative, the pharmaceutical formulation is a lyophilizate.

In one embodiment, the pharmaceutical formulation comprises 5.5 wt.-% to 6.8 wt.-% PEG-ADM, wherein the concentration is based on the total weight of the pharmaceutical formulation. In one alternative, the pharmaceutical formulation is a lyophilizate.

In one embodiment, the pharmaceutical formulation comprises 5.5 wt.-% to 6.5 wt.-% PEG-ADM, wherein the concentration is based on the total weight of the pharmaceutical formulation. In one alternative, the pharmaceutical formulation is a lyophilizate.

In one embodiment, the pharmaceutical formulation comprises 6 wt.-% to 6.5 wt.-% PEG-ADM, wherein the concentration is based on the total weight of the pharmaceutical formulation. In one alternative, the pharmaceutical formulation is a lyophilizate.

### ii. pH-regulator (Component C) - Concentrations (wt.-%)

In one embodiment the pH regulator (component c) is selected from the embodiments disclosed under section I.iii. above.

In one embodiment, the pharmaceutical formulation comprises 0.1 wt.-% to 25 wt.-% of a pH regulator, wherein the concentration is based on the total weight of the pharmaceutical formulation.

In one embodiment, the pharmaceutical formulation comprises 1 wt.-% to 15 wt.-% of a pH regulator, wherein the concentration is based on the total weight of the pharmaceutical formulation.

In one embodiment, the pharmaceutical formulation comprises 3 wt.-% to 12 wt.-% of a pH regulator, wherein the concentration is based on the total weight of the pharmaceutical formulation.

In one embodiment, the pharmaceutical formulation comprises 5 wt.-% to 10 wt.-% of a pH regulator, wherein the concentration is based on the total weight of the pharmaceutical formulation.

In one embodiment, the pharmaceutical formulation comprises 7 wt.-% to 10 wt.-% of a pH regulator, wherein the concentration is based on the total weight of the pharmaceutical formulation.

In one embodiment, the pharmaceutical formulation comprises 8.5 wt.-% to 10 wt.-% of a pH regulator, wherein the concentration is based on the total weight of the pharmaceutical formulation.

In one embodiment, the pharmaceutical formulation comprises 8.5 wt.-% to 9.5 wt.-% of a pH regulator, wherein the concentration is based on the total weight of the pharmaceutical formulation.

In one embodiment, the pharmaceutical formulation comprises 8.8 wt.-% to 9.5 wt.-% of a pH regulator, wherein the concentration is based on the total weight of the pharmaceutical formulation.

In one embodiment, the pharmaceutical formulation comprises 9 wt.-% to 9.5 wt.-% of a pH regulator, wherein the concentration is based on the total weight of the pharmaceutical formulation.

In one embodiment, the pharmaceutical formulation comprises

| | |
|---|---|
| 0.01 wt.-% to 5 wt.-% | Sodium hydroxide |
| 0.01 wt.-% to 5 wt.-% | Sodium chloride |
| 0.01 wt.-% to 5 wt.-% | Hydrochloric acid |

wherein the concentration is based on the total weight of the pharmaceutical formulation.

In one embodiment, the pharmaceutical formulation comprises

| | |
|---|---|
| 3 wt.-% to 4 wt.-% | Sodium hydroxide |
| 3 wt.-% to 4 wt.-% | Sodium chloride |
| 0.5 wt.-% to 1 wt.-% | Hydrochloric acid |

wherein the concentration is based on the total weight of the pharmaceutical formulation.

In one embodiment, the pharmaceutical formulation comprises

| | |
|---|---|
| 1 wt.-% to 15 wt.-% | Citric acid |
| 0.01 wt.-% to 5 wt.-% | Sodium hydroxide |
| 0.01 wt.-% to 5 wt.-% | Sodium chloride |
| 0.01 wt.-% to 5 wt.-% | Hydrochloric acid |

wherein the concentration is based on the total weight of the pharmaceutical formulation.

In one embodiment, the pharmaceutical formulation comprises

| | |
|---|---|
| 8.5 wt.-% to 10 wt.-% | Citric acid |
| 3 wt.-% to 4 wt.-% | Sodium hydroxide |
| 3 wt.-% to 4 wt.-% | Sodium chloride |
| 0.5 wt.-% to 1 wt.-% | Hydrochloric acid |

wherein the concentration is based on the total weight of the pharmaceutical formulation.

In a further alternative of all embodiments disclosed in this section II.ii, the pharmaceutical can be a lyophilizate.

### iii. Osmolarity Regulator (Component D) - Concentrations (wt.-%)

In one embodiment, the pharmaceutical formulation can comprise an osmolarity regulator. In one embodiment the osmolarity regulator (component d) is selected from the embodiments disclosed under section I.iv. above. In a further alternative of all embodiments disclosed in this section II.iii, the pharmaceutical can be a lyophilizate.

### iv. Trehalose - (Component E) - Concentrations (wt.-%)

In one embodiment the trehalose (component e) is selected from the embodiments disclosed under section I.v. above. The wt.-% of trehalose is calculated on basis of the dry trehalose.

In one embodiment, the pharmaceutical formulation comprises 60 wt.-% to 98 wt.-% of trehalose, wherein the concentration is based on the total weight of the pharmaceutical formulation.In one embodiment, the pharmaceutical formulation comprises 65 wt.-% to 95 wt.-% of trehalose, wherein the concentration is based on the total weight of the pharmaceutical formulation.In one embodiment, the pharmaceutical formulation comprises 70 wt.-% to 92 wt.-% of trehalose, wherein the concentration is based on the total weight of the pharmaceutical formulation.In one embodiment, the pharmaceutical formulation comprises 70 wt.-% to 85 wt.-% of trehalose, wherein the concentration is based on the total weight of the pharmaceutical formulation.In one embodiment, the pharmaceutical formulation comprises 70 wt.-% to 80 wt.-% of trehalose, wherein the concentration is based on the total weight of the pharmaceutical formulation.In one embodiment, the pharmaceutical formulation comprises 75 wt.-% to 80 wt.-% of trehalose, wherein the concentration is based on the total weight of the pharmaceutical formulation.In one embodiment, the pharmaceutical formulation comprises 75 wt.-% to 79 wt.-% of trehalose, wherein the concentration is based on the total weight of the pharmaceutical formulation.In one embodiment, the pharmaceutical formulation comprises 75 wt.-% to 78 wt.-% of trehalose, wherein the concentration is based on the total weight of the pharmaceutical formulation.In one embodiment, the pharmaceutical formulation comprises 76 wt.-% to 78 wt.-% of trehalose, wherein the concentration is based on the total weight of the pharmaceutical formulation.In one embodiment, the pharmaceutical formulation comprises 76.5 wt.-% to 78 wt.-% of trehalose, wherein the concentration is based on the total weight of the pharmaceutical formulation.In a further alternative of all embodiments disclosed in this section II.iv, the pharmaceutical can be a lyophilizate.

### v. Further Embodiments

In one embodiment, the pharmaceutical formulation comprises

| | |
|---|---|
| 1 wt.-% to 15 wt.-% | PEG-ADM |
| 1 wt.-% to 15 wt.-% | Citric acid anhydrous |
| 60 wt.-% to 98 wt.-% | Trehalose |

wherein the concentration is based on the total weight of the pharmaceutical formulation.

In one embodiment, the pharmaceutical formulation comprises

| | |
|---|---|
| 5.5 wt.-% to 7 wt.-% | PEG-ADM |
| 7 wt.-% to 10 wt.-% | Citric acid anhydrous |
| 70 wt.-% to 80 wt.-% | Trehalose |

wherein the concentration is based on the total weight of the pharmaceutical formulation.

In one embodiment, the pharmaceutical formulation comprises

| | |
|---|---|
| 5.5 wt.-% to 7 wt.-% | PEG-ADM |
| 8.5 wt.-% to 10 wt.-% | Citric acid anhydrous |
| 70 wt.-% to 80 wt.-% | Trehalose |

wherein the concentration is based on the total weight of the pharmaceutical formulation.

In one embodiment, the pharmaceutical formulation comprises

| | |
|---|---|
| 5.5 wt.-% to 7 wt.-% | PEG-ADM |
| 8.5 wt.-% to 9.5 wt.-% | Citric acid anhydrous |
| 70 wt.-% to 80 wt.-% | Trehalose |

wherein the concentration is based on the total weight of the pharmaceutical formulation.

In one embodiment, the pharmaceutical formulation comprises

| | |
|---|---|
| 5.5 wt.-% to 7 wt.-% | PEG-ADM |
| 8.5 wt.-% to 10 wt.-% | Citric acid anhydrous |
| 75 wt.-% to 78 wt.-% | Trehalose |

wherein the concentration is based on the total weight of the pharmaceutical formulation.

In one embodiment, the pharmaceutical formulation comprises

| | |
|---|---|
| 5.5 wt.-% to 7 wt.-% | PEG-ADM |
| 8.5 wt.-% to 9.5 wt.-% | Citric acid anhydrous |
| 75 wt.-% to 78 wt.-% | Trehalose |

wherein the concentration is based on the total weight of the pharmaceutical formulation.

In one embodiment, the pharmaceutical formulation comprises

| | |
|---|---|
| 6 wt.-% to 6.5 wt.-% | PEG-ADM |
| 8.8 wt.-% to 9.5 wt.-% | Citric acid anhydrous |
| 75 wt.-% to 78 wt.-% | Trehalose |

wherein the concentration is based on the total weight of the pharmaceutical formulation.

In one embodiment, the pharmaceutical formulation comprises

| | |
|---|---|
| 6 wt.-% to 6.5 wt.-% | PEG-ADM |
| 8.8 wt.-% to 9.5 wt.-% | Citric acid anhydrous |
| 76 wt.-% to 78 wt.-% | Trehalose |

wherein the concentration is based on the total weight of the pharmaceutical formulation.

In one embodiment, the pharmaceutical formulation comprises

| | |
|---|---|
| 1 wt.-% to 15 wt.-% | Citric acid anhydrous |
| 0.01 wt.-% to 5 wt.-% | Sodium hydroxide |
| 0.01 wt.-% to 5 wt.-% | Sodium chloride |
| 0.01 wt.-% to 5 wt.-% | Hydrochloric acid |

wherein the concentration is based on the total weight of the pharmaceutical formulation.

In one embodiment, the pharmaceutical formulation comprises

| | |
|---|---|
| 8.5 wt.-% to 10 wt.-% | Citric acid |
| 3 wt.-% to 4 wt.-% | Sodium hydroxide |
| 3 wt.-% to 4 wt.-% | Sodium chloride |
| 0.5 wt.-% to 1 wt.-% | Hydrochloric acid |

wherein the concentration is based on the total weight of the pharmaceutical formulation.

In one embodiment, the pharmaceutical formulation comprises

| | |
|---|---|
| 1 wt.-% to 15 wt.-% | Citric acid |
| 0.01 wt.-% to 5 wt.-% | Sodium hydroxide |
| 0.01 wt.-% to 5 wt.-% | Sodium chloride |
| 0.01 wt.-% to 5 wt.-% | Hydrochloric acid |
| 60 wt.-% to 98 wt.-% | Trehalose |

wherein the concentration is based on the total weight of the pharmaceutical formulation.

In one embodiment, the pharmaceutical formulation comprises

| | |
|---|---|
| 5.5 wt.-% to 7 wt.-% | PEG-ADM |
| 8.5 wt.-% to 10 wt.-% | Citric acid |
| 3 wt.-% to 4 wt.-% | Sodium hydroxide |
| 3 wt.-% to 4 wt.-% | Sodium chloride |
| 0.5 wt.-% to 1 wt.-% | Hydrochloric acid |
| 75 wt.-% to 85 wt.-% | Trehalose |

wherein the concentration is based on the total weight of the pharmaceutical formulation.

In one embodiment, the pharmaceutical formulation comprises

| | |
|---|---|
| 5.5 wt.-% to 7 wt.-% | PEG-ADM |
| 8.5 wt.-% to 10 wt.-% | Citric acid |
| 3 wt.-% to 4 wt.-% | Sodium hydroxide |
| 3 wt.-% to 4 wt.-% | Sodium chloride |
| 0.5 wt.-% to 1 wt.-% | Hydrochloric acid |
| 70 wt.-% to 80 wt.-% | Trehalose |

wherein the concentration is based on the total weight of the pharmaceutical formulation.

In one embodiment, the pharmaceutical formulation comprises

| | |
|---|---|
| 5.5 wt.-% to 7 wt.-% | PEG-ADM |
| 8.5 wt.-% to 9.5 wt.-% | Citric acid |
| 3 wt.-% to 4 wt.-% | Sodium hydroxide |
| 3 wt.-% to 4 wt.-% | Sodium chloride |
| 0.5 wt.-% to 1 wt.-% | Hydrochloric acid |
| 70 wt.-% to 80 wt.-% | Trehalose |

wherein the concentration is based on the total weight of the pharmaceutical formulation.

In one embodiment, the pharmaceutical formulation comprises

| | |
|---|---|
| 6 wt.-% to 6.5 wt.-% | PEG-ADM |
| 8.8 wt.-% to 9.5 wt.-% | Citric acid s |
| 3 wt.-% to 4 wt.-% | Sodium hydroxide |
| 3 wt.-% to 4 wt.-% | Sodium chloride |
| 0.5 wt.-% to 1 wt.-% | Hydrochloric acid |
| 75 wt.-% to 78 wt.-% | Trehalose |

wherein the concentration is based on the total weight of the pharmaceutical formulation.

In one embodiment, the pharmaceutical formulation comprises

| | |
|---|---|
| 6 wt.-% to 6.5 wt.-% | PEG-ADM |
| 8.8 wt.-% to 9.5 wt.-% | Citric acid |
| 3 wt.-% to 4 wt.-% | Sodium hydroxide |
| 3 wt.-% to 4 wt.-% | Sodium chloride |
| 0.5 wt.-% to 1 wt.-% | Hydrochloric acid |
| 76 wt.-% to 78 wt.-% | Trehalose |

wherein the concentration is based on the total weight of the pharmaceutical formulation.

In a further alternative of all embodiments disclosed in this section II.v, the pharmaceutical can be a lyophilizate.

### vi. Method for Preparing the Pharmaceutical Formulation

The invention further provides a method for the preparation of the formulation disclosed in section II, II.i to II.v above.

A method for the preparation of the pharmaceutical formulation comprising the following steps:
step 1. Providing at least components a, c and e; and
step 2. Mixing the components provided in step 1;
step 3: freeze-drying the pharmaceutical formulation obtained after any one of steps 1 and/or 2
whereby the following pharmaceutical formulation as described in any one of the embodiments described in section II, II.i to II.v is obtained.

In one embodiment, the method further comprises step 4 and/ or step 5:
step 4. Adjusting the pH of the pharmaceutical formulation to a pH of 3 to 5; and/or
step 5. Adjusting the osmolarity of the pharmaceutical formulation to an osmotic concentration between 150 - 450 mosmol/1;
wherein step 4 can be carried before, during and/or after step 1, 2 and/or step 5; and/or wherein step 4 can be carried before, during and/or after step 1, 2 and/or step 4.

In one embodiment, the method comprises the following steps
providing an aqueous formulation of PEG-ADM, which comprises citric acid and optionally at least one pH regulator to adjust the pH to 3.5 and 4.5,
optionally followed by concentration of the aqueous formulation of PEG-ADM
freeze-drying of the formulation and
subsequently reconstitution/dilution of the concentrated product by adding a solution of citric acid and/or sodium citrate, optionally at least one pH regulator and an osmolarity regulator and water, and
wherein the pharmaceutical formulation has an osmotic concentration between 150 - 450 mosmol/L; and
wherein the pH of the resulting aqueous formulation is between 3.5 and 4.5.

In one embodiment, the method comprises the following steps
providing an aqueous formulation of PEG-ADM, which comprises citric acid and optionally at least one pH regulator to adjust the pH between 3.5 and 4.5,
providing citric acid and/or sodium citrate, optionally at least one pH regulator and an osmolarity regulator and
mixing the solutions provided, and
wherein the pharmaceutical formulation has an osmotic concentration of between 150 - 450 mosmol/l; and wherein the pH of the resulting aqueous formulation is between 3.5 and 4.5.

In one embodiment, the method further comprises step 6

Step 6 freeze-drying the pharmaceutical formulation obtained after any one of steps 1, 2, 3, 4 and/or 5; wherein step 6 can be carried before, during and/or after step 1, 2, 3, 4 and/or step 5, whereby a lyophilizate is obtained.

In one embodiment, the method further comprises step 7

Step 7 reconstitution of the lyophilizate according to any one of the embodiments as described in any one of the embodiments described in section II, II.i to II.v obtained after any one of steps 1, 2, 3, 4, 5 and/or 6.

The invention also provides a f pharmaceutical formulation according as described in any one of the embodiments in section II obtainable by the method according to any one of the embodiments disclosed in section II.vi.

### III. Liquid Pharmaceutical Formulation

The invention provides for: A liquid pharmaceutical formulation comprising:
a. 0.04 mg/mL to 145 mg/mL of PEG-ADM, wherein the PEG-ADM is a compound according to the general formula (I),

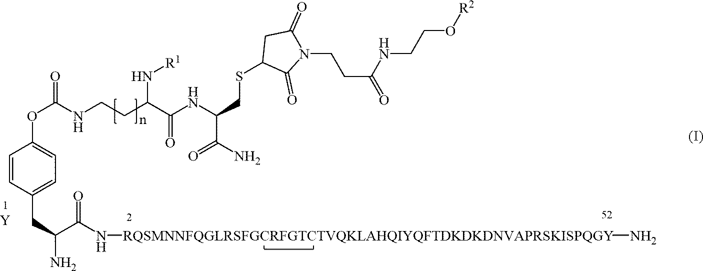

in which
   n represents the number 0, 1, 2 or 3,
   R¹ represents hydrogen, methyl, ethyl, n-propyl or isopropyl,
   R² represents linear or branched PEG 20 kDa to 80 kDa endcapped with a methoxy-group,
   or a hydrate thereof, solvate thereof, salt thereof, pharmaceutically acceptable salt thereof, or the solvates of salts thereof;
   b. a solvent;
   c. a pH regulator;
   d. an osmolarity regulator; and
   e. trehalose;
wherein the presence of the osmolarity regulator (component d) is optional;
wherein the liquid pharmaceutical formulation has a pH between 3 and 5; and
wherein the concentrations of components are based on the total volume of the liquid pharmaceutical formulation.

In one embodiment the liquid pharmaceutical formulation according to the invention is for inhalation and/or inhalative use.

The liquid pharmaceutical formulation comprises at least PEG-ADM (component a), a solvent (component b), a pH regulator (component c), trehalose (component e) and optionally an osmolarity regulator (component d).

These components are described in section I (sections I.i to I.v.) detail above and display embodiments that can be used in the liquid pharmaceutical formulation described in this section III. The embodiments of the concentrations of the respective components comprised in the liquid pharmaceutical formulation are described in section III (sections III.i. to III.ix) in detail below. A method for the preparation of the liquid pharmaceutical formulation is described in section III.x. below.

The concentrations of components are based on the total volume of the liquid pharmaceutical formulation. The liquid pharmaceutical formulation has an osmotic concentration of 150 to 450 mosmol/L. The liquid pharmaceutical formulation has a pH of 3 to 5.

Accordingly, even if not stated specifically in the embodiments of the invention disclosed herein, the following features apply to all embodiments of the liquid pharmaceutical formulation disclosed in this section III.:
when referring to “PEG-ADM” also a compound according to formula (I), a hydrate thereof, solvate thereof, salt thereof, pharmaceutically acceptable salt thereof, or the solvates of salts thereof, are meant;
the concentrations of components a, b, c and d are based on the total volume of the liquid pharmaceutical formulation, and
the liquid pharmaceutical formulation has a pH of 3 to 5
liquid pharmaceutical formulation has an osmotic concentration of 150 to 450 mosmol/L.

Some concentrations are given in “mg/mL”. Mass concentration of solution is expressed as “mg/mL” for “milligram per milliliter”. Here, a solid compound is dissolved in the liquid. For example, if 100 mg of sodium chloride is used to make up a total volume of 100 mL, then a 1 mg/mL solution of sodium chloride has been made. The concentrations of components are based on the total volume of the liquid pharmaceutical formulation.

Moreover, the components c comprised in the liquid pharmaceutical formulation can act also as osmolarity regulator (component d.). This means they can have overlapping functionality. For example, as described in more detail below, a buffer system of citric acid, sodium citrate and/or hydrochloric acid and sodium hydroxide would act as osmolarity regulator as well due to the ions contains in the solution. In that case the components c. and d. are present by one and the same component(s). d. Nevertheless, there are functionalities of the components c and d that overlap. However, these overlapping concentrations are disregarded when calculating the concentrations of the pH regulator or the osmolarity regulator, respectively. The osmolarity regulators are neutral salts, e.g. sodium chloride (NaCl). The pH regulators can contain salts or substances that contribute to osmolarity (e.g. buffer comprising citric acid, sodium citrate and hydrochloric acid comprises in solution sodium ions and chloride ions). The concentration of these contributing salts is not included in the concentration of the osmolarity regulator.

In one embodiment the liquid pharmaceutical formulation according to the invention is a solution. The term “solution” is used as typically in the art. It refers to a homogeneous liquid preparation that contain one or more substances dissolved, i.e., molecularly dispersed, in a suitable solvent and/or mixture of mutually miscible solvents.

In one embodiment the liquid pharmaceutical formulation according to the invention is an aqueous solution. The aqueous solution substantially contains or consists of water as solvent b. “Substantially” here means greater than or equal to 80% by weight, 90% by weight, 95% by weight, 99% by weight or 99.9% by weight, in each case based on the total weight of the overall weight of the liquid pharmaceutical formulation.

In one embodiment the liquid phase of the liquid pharmaceutical formulation according to the invention substantially contains or consists of water. “Substantially” here means greater than or equal to 80% by weight, 90% by weight, 95% by weight, 96% by weight, 97% by weight, 98% by weight, 99% by weight or 99.9% by weight, in each case based on the total weight of the overall weight of the liquid phase.

In one embodiment the liquid pharmaceutical formulation according to the invention is a dispersion. “Dispersions” and/or “disperse systems” are known in principle to a person skilled in the art (cf. “Pharmazeutische Technologie”, Voigt, Deutscher Apotheker Verlag Stuttgart, 2000, pp. 81 ff.). Disperse phases can be classified according to their particle size as follows: molecularly dispersed solution having a particle size of <1 nm (e.g. real solution / fluid phases); colloidally dispersed dissolved having a particle size of greater and/or equal to 1 nm to 1 µm; and coarsely dispersed having a particle size of greater of 1 µm. In one embodiment the liquid pharmaceutical formulation according to the present invention is an aqueous dispersion. The term “aqueous” is defined above and refers to the liquid phase of the dispersion.

In one embodiment the PEG-ADM (component a) is selected from the embodiments disclosed under section I.i. above. In one embodiment the solvent (component b) is selected from the embodiments disclosed under section I.ii. above. In one embodiment the pH regulator (component c) is selected from the embodiments disclosed under section I.iii. above. In one embodiment the osmolarity regulator (component d) is selected from the embodiments disclosed under section I.iv. above. In one embodiment the trehalose (component e) is selected from the embodiments disclosed under section I.v. above.

In one embodiment the PEG-ADM (component a) is selected from the embodiments disclosed under section I.i. above, the solvent (component b) is selected from the embodiments disclosed under section I.ii. above, the pH regulator (component c) is selected from the embodiments disclosed under section I.iii. above, and/or the trehalose (component e) is selected from the embodiments disclosed under section I.v. above.

In one embodiment the PEG-ADM (component a) is selected from the embodiments disclosed under section I.i. above, the solvent (component b) is selected from the embodiments disclosed under section I.ii. above, the pH regulator (component c) is selected from the embodiments disclosed under section I.iii. above, the osmolarity regulator (component d) is selected from the embodiments disclosed under section I.iv. above, and/or the trehalose (component e) is selected from the embodiments disclosed under section I.v. above.

In one embodiment the PEG-ADM (component a) is selected from the embodiments disclosed under section I.i. above, the solvent (component b) is selected from the embodiments disclosed under section I.ii. above, the pH regulator (component c) is selected from the embodiments disclosed under section I.iii. above, and the trehalose (component e) is selected from the embodiments disclosed under section I.v. above.

In one embodiment the PEG-ADM (component a) is selected from the embodiments disclosed under section I.i. above, the solvent (component b) is selected from the embodiments disclosed under section I.ii. above, the pH regulator (component c) is selected from the embodiments disclosed under section I.iii. above, the osmolarity regulator (component d) is selected from the embodiments disclosed under section I.iv. above, and the trehalose (component e) is selected from the embodiments disclosed under section I.v. above.

### i. PEG-ADM (component A) - Concentrations (mg/mL)

The liquid pharmaceutical formulation according to the invention comprises 0.04 mg/mL to 145 mg/mL of PEG-ADM. The concentration of component a is based on the total volume of the liquid pharmaceutical formulation.

As explained above, PEG-ADM acts as a prodrug and is released from PEG-ADM (cf. WO 2013/064508 A1). In therapy, the amount of ADM comprised in a medicament and/or the ADM released from the prodrug PEG-ADM in the body is an important aspect. Moreover, the respective concentration or amount of ADM comprised in a certain amount of PEG-ADM can widely vary depending on the length of the PEG chain. The length of the PEG chain has an impact on the weight of the PEG-ADM, and, thus, on the amount of PEG-ADM that is needed to provide for a certain concentration of ADM. For example, for a PEG-ADM according to formula (I), wherein R2 comprises a PEG 20 kDa endcapped with a methoxy-group, approximately 1 mg ADM is comprised in approximately 4.4 mg PEG-ADM. For example, for a PEG-ADM according to formula (I), wherein R2 represents linear PEG 40 kDa endcapped with a methoxy-group (cf. compound according to formula (Ia)), approximately 1 mg ADM is comprised in approximately 7.7 mg PEG-ADM. For example, for a PEG-ADM according to formula (I), wherein R2 comprises a PEG 80 kDa endcapped with a methoxy-group, approximately 1 mg ADM is comprised in approximately 14.35 mg PEG-ADM. Therefore, the concentrations given for PEG-ADM herein are approximations.

In one embodiment the liquid pharmaceutical formulation according to the invention comprises 0.077 mg/mL to 77 mg/mL of PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 0.77 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 0.385 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 3.85 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 7.7 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 2.31 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 3.85 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 7.7 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 0.385 mg/mL to 38.5 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 0.77 mg/mL to 38.5 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 0.77 mg/mL to 21.3 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 0.77 mg/mL to 7.7 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 2.31 mg/mL to 7.7 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 2.31 mg/mL to 3.85 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 3.08 mg/mL to 23.1 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 3.08 mg/mL to 77 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 3.08 mg/mL to 23.1 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 3.08 mg/mL to 7.7 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 7.7 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 6.16 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 4.62 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 3.85 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 00.37 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment the liquid pharmaceutical formulation according to the invention comprises 2.31 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (Ia), a hydrate thereof, solvate thereof, salt thereof, pharmaceutically acceptable salt thereof, or the solvates of salts thereof.

In one embodiment, the liquid pharmaceutical formulation comprises approximately 0.044 mg/mL to 44 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 20 kDa.

In one embodiment, the liquid pharmaceutical formulation comprises approximately 0.22 mg/mL to 22 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 20 kDa.

In one embodiment, the liquid pharmaceutical formulation comprises approximately 0.44 mg/mL to 13.2 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 20 kDa.

In one embodiment, the liquid pharmaceutical formulation comprises approximately 0.44 mg/mL to 4.4 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 20 kDa.

In one embodiment, the liquid pharmaceutical formulation comprises approximately 1.3 mg/mL to 2.2 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 20 kDa.

In one embodiment, the liquid pharmaceutical formulation comprises approximately 0.14 mg/mL to 144 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 80 kDa.

In one embodiment, the liquid pharmaceutical formulation comprises approximately 0.7 mg/mL to 71.7 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 80 kDa.

In one embodiment, the liquid pharmaceutical formulation comprises approximately 1.4 mg/mL to 43 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 80 kDa.

In one embodiment, the liquid pharmaceutical formulation comprises approximately 1.4 mg/mL to 14.3 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 80 kDa.

In one embodiment, the liquid pharmaceutical formulation comprises approximately 4.3 mg/mL to 7.2 mg/mL PEG-ADM, wherein the PEG-ADM is a compound according to the general formula (I) as defined in any one of the embodiments disclosed for PEG-ADM herein, a hydrate thereof, solvate thereof, salt thereof, pharmaceutically acceptable salt thereof, or the solvates of salts thereof. In one alternative of this embodiment, the compound is a compound according to formula (I), a hydrate thereof, solvate thereof, salt thereof, pharmaceutically acceptable salt thereof, or the solvates of salts thereof, wherein R2 represents a linear or branched PEG 80 kDa.

In one embodiment the liquid pharmaceutical formulation according to the invention comprises 0.4 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the liquid pharmaceutical formulation according to the invention comprises 0.6 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the liquid pharmaceutical formulation according to the invention comprises 2.464 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the liquid pharmaceutical formulation according to the invention comprises 2.5 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the liquid pharmaceutical formulation according to the invention comprises 3.696 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the liquid pharmaceutical formulation according to the invention comprises 3.7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the liquid pharmaceutical formulation according to the invention comprises 7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the liquid pharmaceutical formulation according to the invention comprises 10.5 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment, the liquid pharmaceutical formulation comprises approximately 0.4 mg/mL to 10.5 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof. In one embodiment, the liquid pharmaceutical formulation comprises approximately 0.4 mg/mL to 7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof. In one embodiment, the liquid pharmaceutical formulation comprises approximately 0.4 mg/mL to 3.7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof. In one embodiment, the liquid pharmaceutical formulation comprises approximately 0.4 mg/mL to 3.696 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof. In one embodiment, the liquid pharmaceutical formulation comprises approximately 0.4 mg/mL to 2.5 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof. In one embodiment, the liquid pharmaceutical formulation comprises approximately 0.4 mg/mL to 2.464 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof. In one embodiment, the liquid pharmaceutical formulation comprises approximately 0.4 mg/mL to 0.6 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment, the liquid pharmaceutical formulation comprises approximately 0.6 mg/mL to 10.5 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof. In one embodiment, the liquid pharmaceutical formulation comprises approximately 0.6 mg/mL to 7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof. In one embodiment, the liquid pharmaceutical formulation comprises approximately 0.6 mg/mL to 3.7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof. In one embodiment, the liquid pharmaceutical formulation comprises approximately 0.6 mg/mL to 3.696 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof. In one embodiment, the liquid pharmaceutical formulation comprises approximately 0.6 mg/mL to 2.5 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof.

In one embodiment the liquid pharmaceutical formulation according to the invention comprises a compound according to the general formula (I) or formula (Ia), a hydrate thereof, solvate thereof, salt thereof, pharmaceutically acceptable salt thereof, or the solvates of salts thereof, wherein the concentration of the PEG-ADM is selected from 0.4 mg/mL, 0.6 mg/mL, 2.464 mg/mL, 2.5 mg/mL, 3.696 mg/mL, 3.7 mg/mL, 7 mg/mL, and 10.5 mg/mL.

In one embodiment the liquid pharmaceutical formulation according to the invention comprises a compound according to the general formula (I) or formula (Ia), a hydrate thereof, solvate thereof, salt thereof, pharmaceutically acceptable salt thereof, or the solvates of salts thereof, wherein the concentration of the PEG-ADM is selected from 0.4 mg/mL, 0.6 mg/mL, 2.464 mg/mL, 3.696 mg/mL, 7 mg/mL, and 10.5 mg/mL.

In one embodiment the pharmaceutical formulation according to the invention comprises a PEG-ADM according to formula (I) or formula (Ia), wherein the ADM concentration comprised in the PEG-ADM is selected from 0.0779 mg/mL, 0.48 mg/mL and 1.363 mg/mL.

In one embodiment the pharmaceutical formulation according to the invention comprises a PEG-ADM according to formula (I) or formula (Ia), wherein the ADM concentration comprised in the PEG-ADM is selected from 0.077 mg/mL, 0.48 mg/mL and 1.36 mg/mL.

In one embodiment the pharmaceutical formulation according to the invention comprises a PEG-ADM according to formula (I) or formula (Ia), wherein the ADM concentration comprised in the PEG-ADM is selected from 0.078 mg/mL, 0.48 mg/mL and 1.36 mg/mL.

### ii. Solvent (Component B) in Liquid Pharmaceutical Formulation

The liquid pharmaceutical formulation according to the invention comprises a solvent. The term “solvent” is used as typically in the art. The terms “solvent” and “component b” are synonyms. The term solvent refers to pure solvents and/or to mixtures of different solvents.

In one embodiment of the liquid pharmaceutical formulation according to the invention, the solvent comprises water. In one embodiment of the liquid pharmaceutical formulation according to the invention, the solvent consists of water. In one embodiment the solvent can be an isotonic, hypertonic or hypotonic sodium chloride solution.

### iii. pH-regulator (Component C) - Concentrations (mg/mL)

In one embodiment, the liquid pharmaceutical formulation comprises 0.1 mg/mL to 250 mg/mL of the pH regulator. In one embodiment, the liquid pharmaceutical formulation comprises 0.3 mg/mL to 250 mg/mL of the pH regulator. In one embodiment, the liquid pharmaceutical formulation comprises 0.5 mg/mL to 100 mg/mL of the pH regulator. In one embodiment, the liquid pharmaceutical formulation comprises 0.9 mg/mL to 90 mg/mL of the pH regulator. In one embodiment, the liquid pharmaceutical formulation comprises 2.5 mg/mL to 46 mg/mL of the pH regulator. In one embodiment, the liquid pharmaceutical formulation comprises 7.8 mg/mL to 29 mg/mL of the pH regulator. In one embodiment, the liquid pharmaceutical formulation comprises 12.5 mg/mL to 19 mg/mL of the pH regulator. In one embodiment, the liquid pharmaceutical formulation comprises 0.01 mg/mL to 100 mg/mL of the pH regulator. In one embodiment, the liquid pharmaceutical formulation comprises 0.1 mg/mL to 50 mg/mL of the pH regulator. In one embodiment, the liquid pharmaceutical formulation comprises 0.5 mg/mL to 25 mg/mL of the pH regulator. In one embodiment, the liquid pharmaceutical formulation comprises 0.8 mg/mL to 15 mg/mL of the pH regulator. In one embodiment, the liquid pharmaceutical formulation comprises 1.5 mg/mL to 9 mg/mL of the pH regulator.

The concentration of component c. is based on the total volume of the liquid pharmaceutical formulation.

In one embodiment the liquid pharmaceutical formulation comprises 0.1 mg/mL to 100 mg/mL citric acid, a salt of citric acid, pharmaceutical acceptable salt of citric acid, a derivative of citric acid and/or mixtures thereof. In one embodiment the liquid pharmaceutical formulation comprises 0.3 mg/mL to 30 mg/mL citric acid, a salt of citric acid, pharmaceutical acceptable salt of citric acid, a derivative of citric acid and/or mixtures thereof. In one embodiment the liquid pharmaceutical formulation comprises 1 mg/mL to 15 mg/mL citric acid, a salt of citric acid, pharmaceutical acceptable salt of citric acid, a derivative of citric acid and/or mixtures thereof. In one embodiment the liquid pharmaceutical formulation comprises 2 mg/mL to 10 mg/mL citric acid, a salt of citric acid, pharmaceutical acceptable salt of citric acid, a derivative of citric acid and/or mixtures thereof. In one embodiment the liquid pharmaceutical formulation comprises 4 mg/mL to 7 mg/mL citric acid, a salt of citric acid, pharmaceutical acceptable salt of citric acid, a derivative of citric acid and/or mixtures thereof. In an alternative of the embodiments listed before, the salt of citric acid, pharmaceutical acceptable salt of citric acid, a derivative of citric acid and/or mixtures thereof, are selected from citric acid anhydrous, sodium citrate and citric acid monohydrate.

In one embodiment the liquid pharmaceutical formulation comprises 0.01 mg/mL to 50 mg/mL sodium hydroxide. In one embodiment the liquid pharmaceutical formulation comprises 0.1 mg/mL to 10 mg/mL sodium hydroxide. In one embodiment the liquid pharmaceutical formulation comprises 0.5 mg/mL to 6 mg/mL sodium hydroxide. In one embodiment the liquid pharmaceutical formulation comprises 0.8 mg/mL to 4 mg/mL sodium hydroxide. In one embodiment the liquid pharmaceutical formulation comprises 1.5 mg/mL to 3 mg/mL sodium hydroxide.

In one embodiment the liquid pharmaceutical formulation comprises 0.1 mg/mL to 100 mg/mL hydrochloric acid. In one embodiment the liquid pharmaceutical formulation comprises 0.5 mg/mL to 50 mg/mL hydrochloric acid. In one embodiment the liquid pharmaceutical formulation comprises 1 mg/mL to 25 mg/mL hydrochloric acid. In one embodiment the liquid pharmaceutical formulation comprises 5 mg/mL to 15 mg/mL hydrochloric acid. In one embodiment the liquid pharmaceutical formulation comprises 7 mg/mL to 9 mg/mL hydrochloric acid. In one alternative of these embodiments, the hydrochloric acid is or comprises hydrochloric acid 10% (m/V).

In one embodiment, the liquid pharmaceutical formulation comprises as component c the following mixture of pH regulators
0.1 mg/mL to 100 mg/mL citric acid, a salt of citric acid, pharmaceutical acceptable salt of citric acid, a derivative of citric acid and/or mixtures thereof;
0.01 mg/mL to 50 mg/mL sodium hydroxide; and
0.1 mg/mL to 100 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises as component b the following mixture of pH regulators
0.3 mg/mL to 30 mg/mL citric acid, a salt of citric acid, pharmaceutical acceptable salt of citric acid, a derivative of citric acid and/or mixtures thereof;
0.1 mg/mL to 10 mg/mL sodium hydroxide; and
0.5 mg/mL to 50 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises as component b the following mixture of pH regulators
1 mg/mL to 15 mg/mL citric acid, a salt of citric acid, pharmaceutical acceptable salt of citric acid, a derivative of citric acid and/or mixtures thereof;
0.5 mg/mL to 6 mg/mL sodium hydroxide; and
1 mg/mL to 25 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises as component b the following mixture of pH regulators
2 mg/mL to 10 mg/mL citric acid, a salt of citric acid, pharmaceutical acceptable salt of citric acid, a derivative of citric acid and/or mixtures thereof;
0.8 mg/mL to 4 mg/mL sodium hydroxide; and
5 mg/mL to 15 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises as component b the following mixture of pH regulators
4 mg/mL to 7 mg/mL citric acid, a salt of citric acid, pharmaceutical acceptable salt of citric acid, a derivative of citric acid and/or mixtures thereof;
1.5 mg/mL to 3 mg/mL sodium hydroxide; and
7 mg/mL to 9 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises
0.077 mg/mL to 77 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
0.1 mg/mL to 100 mg/mL citric acid;
0.01 mg/mL to 50 mg/mL sodium hydroxide;
0.1 mg/mL to 100 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises
0.385 mg/mL to 3.85 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
0.3 mg/mL to 30 mg/mL citric acid;
0.1 mg/mL to 10 mg/mL sodium hydroxide;
0.5 mg/mL to 50 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises
0.77 mg/mL to 23.1 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
1 mg/mL to 15 mg/mL citric acid;
0.5 mg/mL to 6 mg/mL sodium hydroxide;
1 mg/mL to 25 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises
0.77 mg/mL to 7.7 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
2 mg/mL to 10 mg/mL citric acid;
0.8 mg/mL to 4 mg/mL sodium hydroxide; and
5 mg/mL to 15 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises
2.31 mg/mL to 3.85 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
4 mg/mL to 7 mg/mL citric acid;
1.5 mg/mL to 3 mg/mL sodium hydroxide; and
7 mg/mL to 9 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises
0.077 mg/mL to 77 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
0.1 mg/mL to 100 mg/mL citric acid;
0.01 mg/mL to 50 mg/mL sodium hydroxide;
0.1 mg/mL to 100 mg/mL hydrochloric acid;

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises
0.385 mg/mL to 3.85 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
0.3 mg/mL to 30 mg/mL citric acid;
0.1 mg/mL to 10 mg/mL sodium hydroxide;
0.5 mg/mL to 50 mg/mL hydrochloric acid;

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises
0.77 mg/mL to 23.1 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
1 mg/mL to 15 mg/mL citric acid;
0.5 mg/mL to 6 mg/mL sodium hydroxide;
1 mg/mL to 25 mg/mL hydrochloric acid;

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises
0.77 mg/mL to 7.7 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
2 mg/mL to 10 mg/mL citric acid;
0.8 mg/mL to 4 mg/mL sodium hydroxide;
5 mg/mL to 15 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment, the liquid pharmaceutical formulation comprises
2.31 mg/mL to 3.85 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
4 mg/mL to 7 mg/mL citric acid;
1.5 mg/mL to 3 mg/mL sodium hydroxide;
7 mg/mL to 9 mg/mL hydrochloric acid.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment wherein the pH regulator comprises or consists of 0.1 mg/mL to 100 mg/mL citric acid.

In one embodiment wherein the pH regulator comprises or consists of 0.3 mg/mL to 30 mg/mL citric acid.

In one embodiment wherein the pH regulator comprises or consists of 1 mg/mL to 15 mg/mL citric acid.

In one embodiment wherein the pH regulator comprises or consists of 2 mg/mL to 10 mg/mL citric acid.

In one embodiment wherein the pH regulator comprises or consists of 4 mg/mL to 7 mg/mL citric acid.

In one embodiment wherein the pH regulator comprises or consists of wherein the pH regulator comprises or consists of 0.01 mg/mL to 50 mg/mL sodium hydroxide.

In one embodiment wherein the pH regulator comprises or consists of 0.1 mg/mL to 10 mg/mL sodium hydroxide.

In one embodiment wherein the pH regulator comprises or consists of 0.5 mg/mL to 6 mg/mL sodium hydroxide.

In one embodiment wherein the pH regulator comprises or consists of 0.8 mg/mL to 4 mg/mL sodium hydroxide.

In one embodiment wherein the pH regulator comprises or consists of 0.1 mg/mL to 100 mg/mL hydrochloric acid.

In one embodiment wherein the pH regulator comprises or consists of 0.5 mg/mL to 50 mg/mL hydrochloric acid.

In one embodiment wherein the pH regulator comprises or consists of 1 mg/mL to 25 mg/mL hydrochloric acid.

In one embodiment wherein the pH regulator comprises or consists of 5 mg/mL to 15 mg/mL of hydrochloric acid 10% (m/V).

In one embodiment wherein the liquid pharmaceutical formulation comprises two or more pH regulators.

In one embodiment wherein the liquid pharmaceutical formulation comprises three or more pH regulators.

In one embodiment wherein the osmolarity regulator is selected from the group consisting of sodium chloride, citric acid, a salt, pharmaceutical acceptable salt, derivative of citric acid and/or mixtures thereof.

In one embodiment wherein the citric acid is a salt, pharmaceutical acceptable salt, derivative of citric acid is selected from the group consisting of citric acid anhydrous, sodium citrate and citric acid monohydrate.

### Iv. Osmolarity Regulator (Component D) - Concentrations (mg/mL)

The concentration of component d. is based on the total volume of the liquid pharmaceutical formulation.

In one embodiment the liquid pharmaceutical formulation comprises 0.1 mg/mL to 250 mg/mL of the pH regulator. In one embodiment the liquid pharmaceutical formulation comprises 0.3 mg/mL to 250 mg/mL of the pH regulator. In one embodiment the liquid pharmaceutical formulation comprises 0.5 mg/mL to 100 mg/mL of the pH regulator. In one embodiment the liquid pharmaceutical formulation comprises 0.9 mg/mL to 90 mg/mL of the pH regulator. In one embodiment the liquid pharmaceutical formulation comprises 2.5 mg/mL to 46 mg/mL of the pH regulator. In one embodiment the liquid pharmaceutical formulation comprises 7.8 mg/mL to 29 mg/mL of the pH regulator. In one embodiment the liquid pharmaceutical formulation comprises 12.5 mg/mL to 19 mg/mL of the pH regulator. In one embodiment the liquid pharmaceutical formulation comprises 0.01 mg/mL to 100 mg/mL of the pH regulator. In one embodiment the liquid pharmaceutical formulation comprises 0.1 mg/mL to 50 mg/mL of the pH regulator. In one embodiment the liquid pharmaceutical formulation comprises 0.5 mg/mL to 25 mg/mL of the pH regulator. In one embodiment the liquid pharmaceutical formulation comprises 0.8 mg/mL to 15 mg/mL of the pH regulator. In one embodiment the liquid pharmaceutical formulation comprises 1.5 mg/mL to 9 mg/mL of the pH regulator.

### v. Trehalose (Component E) - Concentrations (mg/mL)

The concentration of “component e” or “trehalose” is based on the total volume of the liquid pharmaceutical formulation.

In one embodiment the liquid pharmaceutical formulation comprises 1 mg/mL to 300 mg/mL of trehalose. In one embodiment the liquid pharmaceutical formulation comprises 5 mg/mL to 200 mg/mL of trehalose. In one embodiment the liquid pharmaceutical formulation comprises 10 mg/mL to 100 mg/mL of trehalose. In one embodiment the liquid pharmaceutical formulation comprises 30 mg/mL to 70 mg/mL of trehalose. In one embodiment the liquid pharmaceutical formulation comprises 40 mg/mL to 60 mg/mL of trehalose.

### i. pH of the Liquid Pharmaceutical Formulation

The liquid pharmaceutical formulation according to the invention has a pH of 3 to 5. In one embodiment the liquid pharmaceutical formulation according to the invention formulation has a pH of 3.5 to 4.5. In one embodiment the liquid pharmaceutical formulation according to the invention has a pH of 3 to 4. In one embodiment the liquid pharmaceutical formulation according to the invention has a pH of 3 to 3.5. In one embodiment the liquid pharmaceutical formulation according to the invention has a pH of 3.25 to 3.75. In one embodiment the liquid pharmaceutical formulation according to the invention has a pH of 3.5 to 4. In one embodiment the liquid pharmaceutical formulation according to the invention has a pH of 3 In one embodiment the liquid pharmaceutical formulation according to the invention has a pH of 3.5. In one embodiment the liquid pharmaceutical formulation according to the invention has a pH of 4. In one embodiment the liquid pharmaceutical formulation according to the invention has a pH of 4 In one embodiment the liquid pharmaceutical formulation according to the invention has a pH of 5.

### vii. Osmolar Concentration of the Liquid Pharmaceutical Formulation

In the liquid pharmaceutical formulation according to the invention, the osmolar concentration is between 150 to 450 mosmol/L. The osmolarity is expressed as osmotic concentration of “mosmol/l” or “milliosmole per liter”. In one embodiment the liquid pharmaceutical formulation has an osmotic concentration between 150 to 450 mosmol/l. In one embodiment the liquid pharmaceutical formulation has an osmotic concentration between 200 to 400 mosmol/l. In one embodiment the liquid pharmaceutical formulation has an osmotic concentration between 270 to 330 mosmol/l. In one embodiment the liquid pharmaceutical formulation has an osmotic concentration between 250 to 310 mosmol/l. In one embodiment the liquid pharmaceutical formulation has an osmotic concentration of 300 mosmol/l.

### viii. Viscosity of the Liquid Pharmaceutical Formulation

The liquid pharmaceutical formulation according to the invention ca also be characterized by its viscosity. The unit for viscosity is “millipascal second” or “mPa*s”. The viscosity was determined by an automatic rolling ball viscometer method according to Ph.Eur. 2.2.49 (2018), using an Anton Paar AMVn Automated Microviscometer

In one embodiment the viscosity of the formulation according to the invention is 0.9 to 2.2 mPa*s. In one embodiment the viscosity of the formulation according to the invention is approximately 1 to 2 mPa*s. In one embodiment the viscosity of the formulation according to the invention is approximately 1.05 to 2 mPa*s. In one embodiment the viscosity of the formulation according to the invention is approximately 1.05 to 1.9 mPa*s. In one embodiment the viscosity of the formulation according to the invention is approximately 1.1 to 2 mPa*s. In one embodiment the viscosity of the formulation according to the invention is approximately 1.05 mPa*s. In one embodiment the viscosity of the formulation according to the invention is approximately 1.1 mPa*s. In one embodiment the viscosity of the formulation according to the invention is approximately 1.2 mPa*s. In one embodiment the viscosity of the formulation according to the invention is approximately 1.3 mPa*s. In one embodiment the viscosity of the formulation according to the invention is approximately 1.4 mPa*s. In one embodiment the viscosity of the formulation according to the invention is approximately 1.5 mPa*s. In one embodiment the viscosity of the formulation according to the invention is approximately 1.9 mPa*s. In one embodiment the viscosity of the formulation according to the invention is approximately 2 mPa*s.

### ix. Further Embodiments of the Liquid Pharmaceutical Formulation

In one embodiment the liquid pharmaceutical formulation comprises
0.077 mg/mL to 77 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
0.1 mg/mL to 100 mg/mL citric acid;
0.01 mg/mL to 50 mg/mL sodium hydroxide;
0.1 mg/mL to 100 mg/mL hydrochloric acid;
0.01 mg/mL to 100 mg/mL sodium chloride.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment the liquid pharmaceutical formulation comprises
0.385 mg/mL to 3.85 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
0.3 mg/mL to 30 mg/mL citric acid;
0.1 mg/mL to 10 mg/mL sodium hydroxide;
0.5 mg/mL to 50 mg/mL hydrochloric acid;
0.1 mg/mL to 30 mg/mL sodium chloride.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment the liquid pharmaceutical formulation comprises
0.77 mg/mL to 23.1 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
1 mg/mL to 15 mg/mL citric acid;
0.5 mg/mL to 6 mg/mL sodium hydroxide;
1 mg/mL to 25 mg/mL hydrochloric acid;
0.5 mg/mL to 15 mg/mL sodium chloride.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment the liquid pharmaceutical formulation comprises
0.77 mg/mL to 7.7 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
2 mg/mL to 10 mg/mL citric acid;
0.8 mg/mL to 4 mg/mL sodium hydroxide;
5 mg/mL to 15 mg/mL hydrochloric acid;
2 mg/mL to 10 mg/mL sodium chloride.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment the liquid pharmaceutical formulation comprises
2.31 mg/mL to 3.85 mg/mL PEG-ADM, wherein the PEG-ADM is a compound according to the general formula (I) as defined in any one of the embodiments disclosed herein, or a hydrate thereof, solvate thereof, salt thereof, pharmaceutically acceptable salt thereof, or the solvates of salts thereof, wherein the concentration refers to adrenomedullin comprised in the PEG-ADM in an concentration
4 mg/mL to 7 mg/mL citric acid;
1.5 mg/mL to 3 mg/mL sodium hydroxide;
7 mg/mL to 9 mg/mL hydrochloric acid;
5 mg/mL to 7 mg/mL sodium chloride.

In an alternative of this embodiment, the salt of citric acid, pharmaceutical acceptable salt of citric acid, a derivative of citric acid and/or mixtures thereof, are selected from citric acid anhydrous, sodium citrate and citric acid monohydrate.

In one embodiment the liquid pharmaceutical formulation according to the invention the liquid pharmaceutical formulation comprises
0.01 mg/mL to 10 mg/mL of PEG-ADM, wherein the PEG-ADM is a compound according to formula (I) or (Ia);
solvent;
0.1 mg/mL to 100 mg/mL of citric acid,
0.01 mg/mL to 50 mg/mL of sodium hydroxide,
0.1 mg/mL to 100 mg/mL hydrochloric acid 10% (m/V); and
0.01 mg/mL to 100 mg/mL of sodium chloride,
wherein the concentrations of components are based on the total volume of the liquid pharmaceutical formulation; wherein the aqueous formulation has a pH of 3.5 to 4.5, preferably a pH 3 to 4, morepreferably a pH of 4; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate; wherein optionally the hydrochloric acid is hydrochloric acid 10% (m/V); wherein optionally the solvent is or comprises water.

In one embodiment the liquid pharmaceutical formulation according to the invention the liquid pharmaceutical formulation comprises
0.01 mg/mL to 10 mg/mL of PEG-ADM, wherein the PEG-ADM is a compound according to formula (Ia);
water;
0.1 mg/mL to 100 mg/mL of citric acid,
0.01 mg/mL to 50 mg/mL of sodium hydroxide,
0.1 mg/mL to 100 mg/mL hydrochloric acid 10% (m/V); and
0.01 mg/mL to 100 mg/mL of sodium chloride,
wherein the concentrations of components are based on the total volume of the liquid pharmaceutical formulation; wherein the aqueous formulation has a pH of 3.5 to 4.5; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate.

In one embodiment the liquid pharmaceutical formulation according to the invention the liquid pharmaceutical formulation comprises
0.05 mg/mL to 5 mg/mL of PEG-ADM, wherein the PEG-ADM is a compound according to formula (I) or (Ia);
solvent;
0.3 mg/mL to 30 mg/mL of citric acid,
0.1 mg/mL to 10 mg/mL of sodium hydroxide,
0.5 mg/mL to 50 mg/mL hydrochloric acid; and
0.1 mg/mL to 30 mg/mL of sodium chloride,
wherein the concentrations of components are based on the total volume of the liquid pharmaceutical formulation; wherein the aqueous formulation has a pH of 3.5 to 4.5, preferably a pH 3 to 4, more preferably a pH of 4;wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate; wherein optionally the hydrochloric acid is hydrochloric acid 10% (m/V); wherein optionally the solvent is or comprises water.

In one embodiment the liquid pharmaceutical formulation according to the invention the liquid pharmaceutical formulation comprises
0.05 mg/mL to 5 mg/mL of PEG-ADM, wherein the PEG-ADM is a compound according to formula (Ia);
water;
0.3 mg/mL to 30 mg/mL of citric acid,
0.1 mg/mL to 10 mg/mL of sodium hydroxide,
0.5 mg/mL to 50 mg/mL hydrochloric acid 10% (m/V); and
0.1 mg/mL to 30 mg/mL of sodium chloride,
wherein the concentrations of components are based on the total volume of the liquid pharmaceutical formulation; wherein the aqueous formulation has a pH of 3.5 to 4.5; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate.

In one embodiment the liquid pharmaceutical formulation according to the invention the liquid pharmaceutical formulation comprises
0.1 mg/mL to 3 mg/mL of PEG-ADM, wherein the PEG-ADM is a compound according to formula (I) or (Ia);
solvent;
1 mg/mL to 15 mg/mL of citric acid,
0.5 mg/mL to 6 mg/mL of sodium hydroxide,
1 mg/mL to 25 mg/mL hydrochloric acid and
0.5 mg/mL to 15 of sodium chloride,

wherein the concentrations of components are based on the total volume of the liquid pharmaceutical formulation;
wherein the aqueous formulation has a pH of 3.5 to 4.5, preferably a pH 3 to 4, more preferably a pH of 4; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate; wherein optionally the hydrochloric acid is hydrochloric acid 10% (m/V); wherein optionally the solvent is or comprises water.

In one embodiment the liquid pharmaceutical formulation according to the invention the liquid pharmaceutical formulation comprises
0.1 mg/mL to 3 mg/mL of PEG-ADM, wherein the PEG-ADM is a compound according to formula (Ia);
water;
1 mg/mL to 15 mg/mL of citric acid,
0.5 mg/mL to 6 mg/mL of sodium hydroxide,
1 mg/mL to 25 mg/mL hydrochloric acid 10% (m/V); and
0.5 mg/mL to 15 mg/mL of sodium chloride,
wherein the concentrations of components are based on the total volume of the liquid pharmaceutical formulation; wherein the aqueous formulation has a pH of 3.5 to 4.5; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate.

In one embodiment the liquid pharmaceutical formulation according to the invention the liquid pharmaceutical formulation comprises
0.1 mg/mL to 1 mg/mL of PEG-ADM, wherein the PEG-ADM is a compound according to formula (I) or (Ia); solvent;
2 mg/mL to 10 mg/mL of citric acid,
0.8 mg/mL to 4 mg/mL of sodium hydroxide,
5 mg/mL to 15 mg/mL hydrochloric acid and
2 mg/mL to 10 mg/mL of sodium chloride,
wherein the concentrations of components are based on the total volume of the liquid pharmaceutical formulation; wherein the aqueous formulation has a pH of 3.5 to 4.5, preferably a pH 3 to 4, more preferably a pH of 4; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate; wherein optionally the hydrochloric acid is hydrochloric acid 10% (m/V); wherein optionally the solvent is or comprises water.

In one embodiment the liquid pharmaceutical formulation according to the invention the liquid pharmaceutical formulation comprises
0.1 mg/mL to 1 mg/mL of PEG-ADM, wherein the PEG-ADM is a compound according to formula (Ia);
water;
2 mg/mL to 10 mg/mL of citric acid,
0.8 mg/mL to 4 mg/mL of sodium hydroxide,
5 mg/mL to 15 mg/mL hydrochloric acid 10% (m/V); and
2 mg/mL to 10 mg/mL of sodium chloride,
wherein the concentrations of components are based on the total volume of the liquid pharmaceutical formulation; wherein the aqueous formulation has a pH of 3.5 to 4.5; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate.

In one embodiment the liquid pharmaceutical formulation according to the invention the liquid pharmaceutical formulation comprises
0.3 mg/mL to 0.5 mg/mL of PEG-ADM, wherein the PEG-ADM is a compound according to formula (I) or (Ia);
solvent;
4 mg/mL to 7 mg/mL of citric acid,
1.5 mg/mL to 3 mg/mL of sodium hydroxide,
7 mg/mL to 9 mg/mL hydrochloric acid and
5 mg/mL to 7 mg/mL of sodium chloride,
wherein the concentrations of components are based on the total volume of the liquid pharmaceutical formulation; wherein the aqueous formulation has a pH of 3.5 to 4.5, preferably a pH 3 to 4, more preferably a pH of 4; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate; wherein optionally the hydrochloric acid is hydrochloric acid 10% (m/V); wherein optionally the solvent is or comprises water.

In one embodiment the liquid pharmaceutical formulation according to the invention the liquid pharmaceutical formulation comprises
0.3 mg/mL to 0.5 mg/mL of PEG-ADM, wherein the PEG-ADM is a compound according to formula (Ia);
water
4 mg/mL to 7 mg/mL of citric acid,
1.5 mg/mL to 3 mg/mL of sodium hydroxide,
7 mg/mL to 9 mg/mL hydrochloric acid 10% (m/V); and
5 mg/mL to 7 mg/mL of sodium chloride,
wherein the concentrations of components are based on the total volume of the liquid pharmaceutical formulation; wherein the aqueous formulation has a pH of 3.5 to 4.5; wherein optionally the citric acid, a salt of citric acid, pharmaceutical acceptable salt of citric acid, derivative of citric acid, and/or mixtures thereof is selected from the group consisting of citric acid anhydrous, sodium citrate and citric acid monohydrate.

In one embodiment the liquid pharmaceutical formulation according to the invention the liquid pharmaceutical formulation comprises
0.48 mg/mL of PEG-ADM, wherein the PEG-ADM is a compound according to formula (Ia);
water;
5.38 mg/mL of citric acid anhydrous,
2.24 mg/mL of sodium hydroxide,
8.07 mg/mL hydrochloric acid 10% (m/V); and
6.54 mg/mL of sodium chloride,
wherein the concentrations of components are based on the total volume of the liquid pharmaceutical formulation; wherein the aqueous formulation has a pH of 3.5 to 4.5, preferably a pH 3 to 4, more preferably a pH of 4.

In one embodiment the liquid pharmaceutical formulation according to the invention the liquid pharmaceutical formulation comprises
1 mg/mL of PEG-ADM, wherein the PEG-ADM is a compound according to formula (Ia);
water;
5.4 mg/mL of citric acid anhydrous,
2.2 mg/mL of sodium hydroxide,
8.1 mg/mL hydrochloric acid 10% (m/V); and
6.54 mg/mL of sodium chloride,
wherein the concentrations of components are based on the total volume of the liquid pharmaceutical formulation; wherein the aqueous formulation has a pH of 3.5 to 4.5, preferably a pH 3 to 4, more preferably a pH of 4.

In one embodiment the liquid pharmaceutical formulation according to the invention comprises
PEG-ADM, wherein the PEG-ADM a compound according to the general formula (I) or formula (Ia), a hydrate thereof, solvate thereof, salt thereof, pharmaceutically acceptable salt thereof, or the solvates of salts thereof, wherein the concentration of the PEG-ADM is selected from 0.4 mg/mL, 0.6 mg/mL, 2.464 mg/mL, 3.696 mg/mL, 3.7 mg/mL, 7 mg/mL, and 10.5 mg/mL; and
trehalose in a concentration selected from 13.1 mg/mL, 19.65 mg/mL, 20.33 mg/mL, 30.5 mg/mL, 33. 33 mg/mL, 49.33 mg/mL, 50 mg/mL, 70.6 mg/mL, 74 mg/mL, and 106 mg/mL.

In one embodiment the liquid pharmaceutical formulation according to the invention comprises
PEG-ADM, wherein the PEG-ADM a compound according to the general formula (I) or formula (Ia), a hydrate thereof, solvate thereof, salt thereof, pharmaceutically acceptable salt thereof, or the solvates of salts thereof, wherein the concentration of the PEG-ADM is selected from 2.464 mg/mL; and
trehalose in a concentration selected from 13.1 mg/mL, 20.33 mg/mL, 33.33 mg/mL, 49.33 mg/mL, and 70.6 mg/mL.

In one embodiment the liquid pharmaceutical formulation according to the invention comprises
PEG-ADM, wherein the PEG-ADM a compound according to the general formula (I) or formula (Ia), a hydrate thereof, solvate thereof, salt thereof, pharmaceutically acceptable salt thereof, or the solvates of salts thereof, wherein the concentration of the PEG-ADM is selected from 3.696 mg/mL; and
trehalose in a concentration selected from 19.65 mg/mL, 30.5 mg/mL, 50 mg/mL, 74 mg/mL, and 106 mg/mL.

In one embodiment the liquid pharmaceutical formulation according to the invention comprises
PEG-ADM, wherein the PEG-ADM a compound according to the general formula (I) or formula (Ia), a hydrate thereof, solvate thereof, salt thereof, pharmaceutically acceptable salt thereof, or the solvates of salts thereof, wherein the concentration of the PEG-ADM is selected from 0.4 mg/mL, 2.464 mg/mL, and 7 mg/mL; and
trehalose in a concentration selected from 33.33 mg/mL.

In one embodiment the liquid pharmaceutical formulation according to the invention comprises
PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof, wherein the concentration of the PEG-ADM is selected from 0.6 mg/mL, 3.696 mg/mL, and 10.5 mg/mL; and
trehalose in a concentration selected from 50 mg/mL.

In one embodiment the liquid pharmaceutical formulation according to the invention comprises
0.4 mg/mL to 10.5 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof; and
13.1 mg/mL to 106 mg/mL trehalose.

In one embodiment the liquid pharmaceutical formulation according to the invention comprises
2.464 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof; and
13.1 mg/mL to 70.6 mg/mL trehalose.

In one embodiment the liquid pharmaceutical formulation according to the invention comprises
3.696 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts thereof; and
19.65 mg/mL to 106 mg/mL trehalose.

In one embodiment the liquid pharmaceutical formulation according to the invention comprises
0.4 to 7 mg/mL PEG-ADM, wherein the PEG-ADM is a compound according to the general formula (I) or formula (Ia), a hydrate thereof, solvate thereof, salt thereof, pharmaceutically acceptable salt thereof, or the solvates of salts; and
33.33 mg/mL trehalose.

The embodiments disclosed in this section “Further embodiments” can also have the pH, the osmolar concentration and/or the viscosity as disclosed in sections “pH of the liquid pharmaceutical formulation”, “viscosity of the liquid pharmaceutical formulation” or “osmolar concentration of the liquid pharmaceutical formulation”, respectively.

### x. Method for Preparing the Liquid Pharmaceutical Formulation

One subject of the invention is the preparation of the liquid pharmaceutical formulation according to the invention.

The method comprises at least the following steps
step 1. Providing components a, b, c and d; and
step 2. Mixing the components provided in step 1;
whereby the following liquid pharmaceutical formulation is obtained:
a liquid pharmaceutical formulation comprising:
   a. 0.04 mg/mL to 145 mg/mL of PEG-ADM, wherein the PEG-ADM is a compound according to the general formula (I), 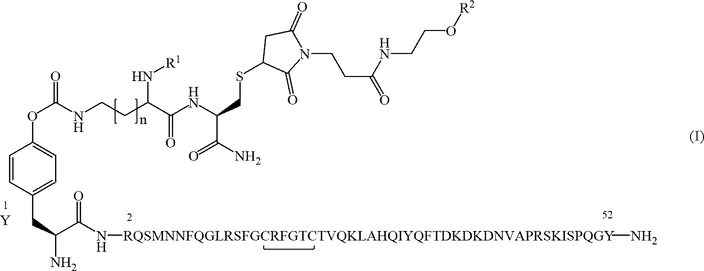
   in which
      n represents the number 0, 1, 2 or 3,
      R¹ represents hydrogen, methyl, ethyl, n-propyl or isopropyl,
      R² represents linear or branched PEG 20 kDa to 80 kDa endcapped with a methoxy-group,
      or a hydrate thereof, solvate thereof, salt thereof, pharmaceutically acceptable salt thereof,
      or the solvates of salts thereof;
   b. a solvent;
   c. a pH regulator; and
   d. an osmolarity regulator;
wherein the liquid pharmaceutical formulation has a pH of 3 to 5; and wherein the osmolar concentration is between 150 to 450 mosmol/L, and wherein the concentrations of components are based on the total volume of the liquid pharmaceutical formulation.

Steps 1 and/or 2 can be conducted separately and/or simultaneously and/or subsequently.

In one embodiment of the method, the PEG-ADM (or component a) is a compound according to any one of the embodiments disclosed under section “PEG-ADM (component a)” above. In one embodiment of the method, the PEG-ADM is a compound according to formula (Ia). In one embodiment of the method, the component b is a compound according to any one of the embodiments disclosed under section “solvent (component b)” above. In one embodiment of the method, the component c is a compound according to any one of the embodiments disclosed under section “pH regulator (component c)” above. In one embodiment of the method, the component d is a compound according to any one of the embodiments disclosed under section “osmolarity regulator (component d)” above. In one embodiment of the method, the liquid pharmaceutical formulation obtained is selected from the embodiments disclosed in the section “Further embodiments of the liquid pharmaceutical formulation”.

In one embodiment of the method, the method further comprises step 3

step 3. adjusting the pH of the liquid pharmaceutical formulation to a pH of 3 to 5,

wherein step 3 can be carried before, during and/or after step 1, 2 and/or step 4.

In one embodiment the pH can be adjusted to any pH disclosed under section “pH of the liquid pharmaceutical formulation”. Steps 1 and/or 2 and/or 3 can be conducted separately and/or simultaneously and/or subsequently. Steps 1 and/or 2 and/or 3 and/or 4 can be conducted separately and/or simultaneously and/or subsequently.

In one embodiment of the method, the method further comprises step 4

step 4. Adjusting the osmolarity of the liquid pharmaceutical formulation to an osmotic concentration of 150 to 450 mosmol/l;

wherein step 4 can be carried before, during and/or after step 1, 2 and/or step 3.

Steps 1 and/or 2 and/or 3 and/or 4 can be conducted separately and/or simultaneously and/or subsequently.

In one embodiment of the method, the method comprises steps 1 to 4 and the liquid pharmaceutical formulation is prepared as follows
providing an aqueous formulation of PEG-ADM, which comprises citric acid and optionally at least one pH regulator to adjust the pH to 3.5 and 4.5,
followed by concentration of the aqueous formulation of PEG-ADM and
subsequently reconstitution/dilution of the concentrated product by adding a solution of citric acid and/or sodium citrate, optionally at least one pH regulator and an osmolarity regulator and water, and
wherein the liquid pharmaceutical formulation has an osmotic concentration of 150 to 450 mosmol/l mosmol/l; and wherein the pH of the resulting aqueous formulation is between 3.5 and 4.5.

In one embodiment of the method, the method comprises steps 1 to 4 and the liquid pharmaceutical formulation is prepared as follows
providing an aqueous formulation of PEG-ADM, which comprises citric acid and optionally at least one pH regulator to adjust the pH to 3.5 and 4.5,
providing citric acid and/or sodium citrate, optionally at least one pH regulator and an osmolarity regulator and
mixing the solutions provided, and
wherein the liquid pharmaceutical formulation has an osmotic concentration of 150 to 450 mosmol/l mosmol/l; and wherein the pH of the resulting aqueous formulation is between 3.5 and 4.5.

The method according to the invention or the embodiments thereof can further comprise step 5:
Step 5 at least partially freezing the liquid pharmaceutical formulation obtained after any one of steps 1, 2, 3 and/or 4.

Steps 1 and/or 2 and/or 3 and/or 4 and/or 5 can be conducted separately and/or simultaneously and/or subsequently.

The invention also provides the liquid pharmaceutical formulation obtainable by the method described in section III.x.

### IV. Pharmaceutical Formulation - Reconstituted Lyophilizate

The invention also provides a pharmaceutical formulation comprising the lyophilizate as disclosed in section II and a solvent as disclosed in any one of sections I, II and/or III. The pharmaceutical formulation comprising the lyophilizate as disclosed in section II and a solvent as disclosed in any one of sections I, II and/or III is called a “reconstituted lyophilizate”. The lyophilizate s reconstituted in the solvent (component b) or “reconstitution medium”. In this section IV, the terms “solvent” and “reconstitution medium” are synonyms.

In one embodiment the solvent is a reconstitution medium. A “reconstitution medium” is a solvent used for solving, dissolving, diluting or dispersing the pharmaceutical formulation, the liquid pharmaceutical formulation and/or a lyophilizate of the aforementioned formulations. In one embodiment a lyophilizate according to any one of the embodiments disclosed herein is solved, dissolved or dispersed by mixing said lyophilizate with the solvent. Here, said lyophilizate is “reconstituted” in the solvent.

In one embodiment, the solvent is water. In one embodiment, the solvent comprises water. In one embodiment the reconstituted lyophilizate is an aqueous solution.

In one embodiment, the solvent is a sodium chloride solution. In one embodiment, the solvent is isotonic sodium chloride solution. In one embodiment, the solvent is hypotonic sodium chloride solution. In one embodiment, the solvent is hypertonic sodium chloride solution. In one embodiment the solvent is a buffer. Embodiments of buffers that can be used in the formulations according to the invention are citrate buffer (pH 3-6.2; pKa 3.3/4.8/6.4), phosphate citrate buffer (pH 2.2-8.0, pKa = 7.2/6.4/2.2), phosphate buffer (pH 2-12; pKa 2.2/6.9/12.3), sodium acetate buffer (pH 3.6-5.6, pKa 4.76), glycine-HCl (pH 2.2-3.6, pKa 2.35), leucine buffer (pH 2-4; pKa 2.3), aspartic acid buffer (pH 3-5; pKa 2.0/3.9), glutamic acid buffer (pH 3-6; pKa 2.2/4.3). Even if not explicitly stated herein, any buffer that is suitable for adjusting the pH to 3 to 5 can be used in the pharmaceutical formulation according to the invention.

In one embodiment the solvent is a mixture of the aforementioned sodium chloride solution and a buffer. In one alternative of this embodiment, the sodium chloride solution is an isotonic sodium chloride solution. In one alternative of this embodiment, the solvent is hypotonic sodium chloride solution. In one alternative of this embodiment, the solvent is hypertonic sodium chloride solution. In one alternative of this embodiment, the buffer is citrate buffer. In one alternative of this embodiment, the buffer is selected from the group consisting of citrate buffer (pH 3-6.2; pKa 3.3/4.8/6.4), phosphate citrate buffer (pH 2.2-8.0, pKa = 7.2/6.4/2.2), phosphate buffer (pH 2-12; pKa 2.2/6.9/12.3), sodium acetate buffer (pH 3.6-5.6, pKa 4.76), glycine-HCl (pH 2.2-3.6, pKa 2.35), leucine buffer (pH 2-4; pKa 2.3), aspartic acid buffer (pH 3-5; pKa 2.0/3.9) glutamic acid buffer (pH 3-6; pKa 2.2/4.3) and/or mixtures thereof.

In one embodiment the PEG-ADM (component a) is selected from the embodiments disclosed under section I.i. above. In one embodiment the solvent (component b) is selected from the embodiments disclosed under section I.ii. above. In one embodiment the pH regulator (component c) is selected from the embodiments disclosed under section I.iii. above. In one embodiment the osmolarity regulator (component d) is selected from the embodiments disclosed under section I.iv. above. In one embodiment the trehalose (component e) is selected from the embodiments disclosed under section I.v. above.

In one embodiment the PEG-ADM (component a) is selected from the embodiments disclosed under section I.i. above, the solvent (component b) is selected from the embodiments disclosed under section I.ii. above, the pH regulator (component c) is selected from the embodiments disclosed under section I.iii. above, and/or the trehalose (component e) is selected from the embodiments disclosed under section I.v. above.

In one embodiment the PEG-ADM (component a) is selected from the embodiments disclosed under section I.i. above, the solvent (component b) is selected from the embodiments disclosed under section I.ii. above, the pH regulator (component c) is selected from the embodiments disclosed under section I.iii. above, the osmolarity regulator (component d) is selected from the embodiments disclosed under section I.iv. above, and/or the trehalose (component e) is selected from the embodiments disclosed under section I.v. above.

In one embodiment the PEG-ADM (component a) is selected from the embodiments disclosed under section I.i. above, the solvent (component b) is selected from the embodiments disclosed under section I.ii. above, the pH regulator (component c) is selected from the embodiments disclosed under section I.iii. above, and the trehalose (component e) is selected from the embodiments disclosed under section I.v. above.

In one embodiment the PEG-ADM (component a) is selected from the embodiments disclosed under section I.i. above, the solvent (component b) is selected from the embodiments disclosed under section I.ii. above, the pH regulator (component c) is selected from the embodiments disclosed under section I.iii. above, the osmolarity regulator (component d) is selected from the embodiments disclosed under section I.iv. above, and the trehalose (component e) is selected from the embodiments disclosed under section I.v. above.

In one embodiment, the reconstituted lyophilizate comprises the lyophilizate as disclosed is any one of the embodiments in section II above.

In one embodiment, the reconstituted lyophilizate is any one of the embodiments of the liquid pharmaceutical formulation as disclosed in section III above.

In one embodiment, the reconstituted lyophilizate comprises PEG-ADM in a concentration according to any one of the embodiments disclosed in section II.i.

In one embodiment, the reconstituted lyophilizate comprises PEG-ADM in a concentration according to any one of the embodiments disclosed in section III.i, wherein the concentrations of components are based on the total volume of the liquid pharmaceutical formulation.

In one embodiment, the reconstituted lyophilizate comprises 0.077 mg/mL to 3.7 mg/mL PEG-ADM.

In one embodiment, the reconstituted lyophilizate comprises 0.077 mg/mL to 3.6 mg/mL PEG-ADM.

In one embodiment, the reconstituted lyophilizate comprises 0.077 mg/mL to 3.5 mg/mL PEG-ADM.

In one embodiment, the reconstituted lyophilizate comprises 0.077 mg/mL to 3.4 mg/mL PEG-ADM.

In one embodiment, the reconstituted lyophilizate comprises 0.077 mg/mL to 3.3 mg/mL PEG-ADM.

In one embodiment, the reconstituted lyophilizate comprises 0.077 mg/mL to 3.2 mg/mL PEG-ADM.

In one embodiment, the reconstituted lyophilizate comprises 0.077 mg/mL to 3.1 mg/mL PEG-ADM.

In one embodiment, the reconstituted lyophilizate comprises 0.077 mg/mL to 3 mg/mL PEG-ADM.

In one embodiment, the reconstituted lyophilizate comprises 0.077 mg/mL to 2.9 mg/mL PEG-ADM.

In one embodiment, the reconstituted lyophilizate comprises 0.077 mg/mL to 2.8 mg/mL PEG-ADM.

In one embodiment, the reconstituted lyophilizate comprises 0.077 mg/mL to 2.7 mg/mL PEG-ADM.

In one embodiment, the reconstituted lyophilizate comprises 0.077 mg/mL to 2.6 mg/mL PEG-ADM.

In one embodiment, the reconstituted lyophilizate comprises 0.077 mg/mL to 2.5 mg/mL PEG-ADM.

In one embodiment, the reconstituted lyophilizate comprises 0.077 mg/mL to 2.4 mg/mL PEG-ADM.

In one embodiment, the reconstituted lyophilizate comprises 0.077 mg/mL to 2.3 mg/mL PEG-ADM.

In one embodiment, the reconstituted lyophilizate comprises 0.077 mg/mL to 2.2 mg/mL PEG-ADM.

In one embodiment, the reconstituted lyophilizate comprises 0.077 mg/mL to 2.1 mg/mL PEG-ADM.

In one embodiment, the reconstituted lyophilizate comprises 0.077 mg/mL to 2 mg/mL PEG-ADM.

In one embodiment, the reconstituted lyophilizate comprises 0.077 mg/mL to 1.9 mg/mL PEG-ADM.

In one embodiment, the reconstituted lyophilizate comprises 0.01 mg/mL to 1.9 mg/mL PEG-ADM.

In one embodiment, the reconstituted lyophilizate comprises 0.077 mg/mL to 3.7 mg/mL PEG-ADM.

In one embodiment, the reconstituted lyophilizate comprises 0.077 mg/mL to 3.6 mg/mL PEG-ADM.

In one embodiment, the reconstituted lyophilizate comprises 0.077 mg/mL to 3.5 mg/mL PEG-ADM.

In one embodiment, the reconstituted lyophilizate comprises 0.077 mg/mL to 3.4 mg/mL PEG-ADM.

In one embodiment, the reconstituted lyophilizate comprises 0.077 mg/mL to 3.3 mg/mL PEG-ADM.

In one embodiment, the reconstituted lyophilizate comprises 0.077 mg/mL to 3.2 mg/mL PEG-ADM.

In one embodiment, the reconstituted lyophilizate comprises 0.077 mg/mL to 3.1 mg/mL PEG-ADM.

In one embodiment, the reconstituted lyophilizate comprises 0.077 mg/mL to 3 mg/mL PEG-ADM.

In one embodiment, the reconstituted lyophilizate comprises 0.077 mg/mL to 2.9 mg/mL PEG-ADM.

In one embodiment, the reconstituted lyophilizate comprises 0.077 mg/mL to 2.8 mg/mL PEG-ADM.

In one embodiment, the reconstituted lyophilizate comprises 0.077 mg/mL to 2.7 mg/mL PEG-ADM.

In one embodiment, the reconstituted lyophilizate comprises 0.077 mg/mL to 2.6 mg/mL PEG-ADM.

In one embodiment, the reconstituted lyophilizate comprises 0.077 mg/mL to 2.5 mg/mL PEG-ADM.

In one embodiment, the reconstituted lyophilizate comprises 0.077 mg/mL to 2.4 mg/mL PEG-ADM.

In one embodiment, the reconstituted lyophilizate comprises 0.077 mg/mL to 2.3 mg/mL PEG-ADM.

In one embodiment, the reconstituted lyophilizate comprises 0.077 mg/mL to 2.2 mg/mL PEG-ADM.

In one embodiment, the reconstituted lyophilizate comprises 0.077 mg/mL to 2.1 mg/mL PEG-ADM.

In one embodiment, the reconstituted lyophilizate comprises 0.077 mg/mL to 2 mg/mL PEG-ADM.

In one embodiment, the reconstituted lyophilizate comprises 0.077 mg/mL to 1.9 mg/mL PEG-ADM.

In one embodiment, the reconstituted lyophilizate comprises 0.077 mg/mL to 1.9 mg/mL PEG-ADM.

In one embodiment, the reconstituted lyophilizate comprises 0.385 mg/mL to 3.7 mg/mL PEG-ADM.

In one embodiment, the reconstituted lyophilizate comprises 0.385 mg/mL to 3.6 mg/mL PEG-ADM.

In one embodiment, the reconstituted lyophilizate comprises 0.385 mg/mL to 3.5 mg/mL PEG-ADM.

In one embodiment, the reconstituted lyophilizate comprises 0.385 mg/mL to 3.4 mg/mL PEG-ADM.

In one embodiment, the reconstituted lyophilizate comprises 0.385 mg/mL to 3.3 mg/mL PEG-ADM.

In one embodiment, the reconstituted lyophilizate comprises 0.385 mg/mL to 3.2 mg/mL PEG-ADM.

In one embodiment, the reconstituted lyophilizate comprises 0.385 mg/mL to 3.1 mg/mL PEG-ADM.

In one embodiment, the reconstituted lyophilizate comprises 0.385 mg/mL to 3 mg/mL PEG-ADM.

In one embodiment, the reconstituted lyophilizate comprises 0.385 mg/mL to 2.9 mg/mL PEG-ADM.

In one embodiment, the reconstituted lyophilizate comprises 0.385 mg/mL to 2.8 mg/mL PEG-ADM.

In one embodiment, the reconstituted lyophilizate comprises 0.385 mg/mL to 2.7 mg/mL PEG-ADM.

In one embodiment, the reconstituted lyophilizate comprises 0.385 mg/mL to 2.6 mg/mL PEG-ADM.

In one embodiment, the reconstituted lyophilizate comprises 0.385 mg/mL to 2.5 mg/mL PEG-ADM.

In one embodiment, the reconstituted lyophilizate comprises 0.385 mg/mL to 2.4 mg/mL PEG-ADM.

In one embodiment, the reconstituted lyophilizate comprises 0.385 mg/mL to 2.3 mg/mL PEG-ADM.

In one embodiment, the reconstituted lyophilizate comprises 0.385 mg/mL to 2.2 mg/mL PEG-ADM.

In one embodiment, the reconstituted lyophilizate comprises 0.385 mg/mL to 2.1 mg/mL PEG-ADM.

In one embodiment, the reconstituted lyophilizate comprises 0.385 mg/mL to 2 mg/mL PEG-ADM.

In one embodiment, the reconstituted lyophilizate comprises 0.385 mg/mL to 1.9 mg/mL PEG-ADM.

In one embodiment, the reconstituted lyophilizate comprises 0.01 mg/mL to 1.9 mg/mL PEG-ADM.

In one embodiment, the reconstituted lyophilizate comprises 0.385 mg/mL to 3.7 mg/mL PEG-ADM.

In one embodiment, the reconstituted lyophilizate comprises 0.385 mg/mL to 3.6 mg/mL PEG-ADM.In one embodiment, the reconstituted lyophilizate comprises 0.385 mg/mL to 3.5 mg/mL PEG-ADM.

In one embodiment, the reconstituted lyophilizate comprises 0.385 mg/mL to 3.4 mg/mL PEG-ADM.

In one embodiment, the reconstituted lyophilizate comprises 0.385 mg/mL to 3.3 mg/mL PEG-ADM.

In one embodiment, the reconstituted lyophilizate comprises 0.385 mg/mL to 3.2 mg/mL PEG-ADM.

In one embodiment, the reconstituted lyophilizate comprises 0.385 mg/mL to 3.1 mg/mL PEG-ADM.

In one embodiment, the reconstituted lyophilizate comprises 0.385 mg/mL to 3 mg/mL PEG-ADM.

In one embodiment, the reconstituted lyophilizate comprises 0.385 mg/mL to 2.9 mg/mL PEG-ADM.

In one embodiment, the reconstituted lyophilizate comprises 0.385 mg/mL to 2.8 mg/mL PEG-ADM.

In one embodiment, the reconstituted lyophilizate comprises 0.385 mg/mL to 2.7 mg/mL PEG-ADM.

In one embodiment, the reconstituted lyophilizate comprises 0.385 mg/mL to 2.6 mg/mL PEG-ADM.

In one embodiment, the reconstituted lyophilizate comprises 0.385 mg/mL to 2.5 mg/mL PEG-ADM.

In one embodiment, the reconstituted lyophilizate comprises 0.385 mg/mL to 2.4 mg/mL PEG-ADM.

In one embodiment, the reconstituted lyophilizate comprises 0.385 mg/mL to 2.3 mg/mL PEG-ADM.

In one embodiment, the reconstituted lyophilizate comprises 0.385 mg/mL to 2.2 mg/mL PEG-ADM.

In one embodiment, the reconstituted lyophilizate comprises 0.385 mg/mL to 2.1 mg/mL PEG-ADM.

In one embodiment, the reconstituted lyophilizate comprises 0.385 mg/mL to 2 mg/mL PEG-ADM.

In one embodiment, the reconstituted lyophilizate comprises 0.385 mg/mL to 1.9 mg/mL PEG-ADM.

In one embodiment, the reconstituted lyophilizate comprises 0.385 mg/mL to 1.9 mg/mL PEG-ADM.

In one embodiment, the reconstituted lyophilizate comprises 0.77 mg/mL to 3.7 mg/mL PEG-ADM.

In one embodiment, the reconstituted lyophilizate comprises 0.77 mg/mL to 3.6 mg/mL PEG-ADM.

In one embodiment, the reconstituted lyophilizate comprises 0.77 mg/mL to 3.5 mg/mL PEG-ADM.

In one embodiment, the reconstituted lyophilizate comprises 0.77 mg/mL to 3.4 mg/mL PEG-ADM.

In one embodiment, the reconstituted lyophilizate comprises 0.77 mg/mL to 3.3 mg/mL PEG-ADM.

In one embodiment, the reconstituted lyophilizate comprises 0.77 mg/mL to 3.2 mg/mL PEG-ADM.

In one embodiment, the reconstituted lyophilizate comprises 0.77 mg/mL to 3.1 mg/mL PEG-ADM.

In one embodiment, the reconstituted lyophilizate comprises 0.77 mg/mL to 3 mg/mL PEG-ADM.

In one embodiment, the reconstituted lyophilizate comprises 0.77 mg/mL to 2.9 mg/mL PEG-ADM.

In one embodiment, the reconstituted lyophilizate comprises 0.77 mg/mL to 2.8 mg/mL PEG-ADM.

In one embodiment, the reconstituted lyophilizate comprises 0.77 mg/mL to 2.7 mg/mL PEG-ADM.

In one embodiment, the reconstituted lyophilizate comprises 0.77 mg/mL to 2.6 mg/mL PEG-ADM.

In one embodiment, the reconstituted lyophilizate comprises 0.77 mg/mL to 2.5 mg/mL PEG-ADM.

In one embodiment, the reconstituted lyophilizate comprises 0.77 mg/mL to 2.4 mg/mL PEG-ADM.

In one embodiment, the reconstituted lyophilizate comprises 0.77 mg/mL to 2.3 mg/mL PEG-ADM.

In one embodiment, the reconstituted lyophilizate comprises 0.77 mg/mL to 2.2 mg/mL PEG-ADM.

In one embodiment, the reconstituted lyophilizate comprises 0.77 mg/mL to 2.1 mg/mL PEG-ADM.

In one embodiment, the reconstituted lyophilizate comprises 0.77 mg/mL to 2 mg/mL PEG-ADM.

In one embodiment, the reconstituted lyophilizate comprises 0.77 mg/mL to 1.9 mg/mL PEG-ADM.

In one embodiment, the reconstituted lyophilizate comprises 0.01 mg/mL to 1.9 mg/mL PEG-ADM.

In one embodiment, the reconstituted lyophilizate comprises 0.77 mg/mL to 3.7 mg/mL PEG-ADM.

In one embodiment, the reconstituted lyophilizate comprises 0.77 mg/mL to 3.6 mg/mL PEG-ADM.

In one embodiment, the reconstituted lyophilizate comprises 0.77 mg/mL to 3.5 mg/mL PEG-ADM.

In one embodiment, the reconstituted lyophilizate comprises 0.77 mg/mL to 3.4 mg/mL PEG-ADM.

In one embodiment, the reconstituted lyophilizate comprises 0.77 mg/mL to 3.3 mg/mL PEG-ADM.

In one embodiment, the reconstituted lyophilizate comprises 0.77 mg/mL to 3.2 mg/mL PEG-ADM.

In one embodiment, the reconstituted lyophilizate comprises 0.77 mg/mL to 3.1 mg/mL PEG-ADM.

In one embodiment, the reconstituted lyophilizate comprises 0.77 mg/mL to 3 mg/mL PEG-ADM.

In one embodiment, the reconstituted lyophilizate comprises 0.77 mg/mL to 2.9 mg/mL PEG-ADM.

In one embodiment, the reconstituted lyophilizate comprises 0.77 mg/mL to 2.8 mg/mL PEG-ADM.

In one embodiment, the reconstituted lyophilizate comprises 0.77 mg/mL to 2.7 mg/mL PEG-ADM.

In one embodiment, the reconstituted lyophilizate comprises 0.77 mg/mL to 2.6 mg/mL PEG-ADM.

In one embodiment, the reconstituted lyophilizate comprises 0.77 mg/mL to 2.5 mg/mL PEG-ADM.

In one embodiment, the reconstituted lyophilizate comprises 0.77 mg/mL to 2.4 mg/mL PEG-ADM.

In one embodiment, the reconstituted lyophilizate comprises 0.77 mg/mL to 2.3 mg/mL PEG-ADM.

In one embodiment, the reconstituted lyophilizate comprises 0.77 mg/mL to 2.2 mg/mL PEG-ADM.

In one embodiment, the reconstituted lyophilizate comprises 0.77 mg/mL to 2.1 mg/mL PEG-ADM.

In one embodiment, the reconstituted lyophilizate comprises 0.77 mg/mL to 2 mg/mL PEG-ADM.

In one embodiment, the reconstituted lyophilizate comprises 0.77 mg/mL to 1.9 mg/mL PEG-ADM.

In one embodiment, the reconstituted lyophilizate comprises 0.77 mg/mL to 1.9 mg/mL PEG-ADM.

In one embodiment, the reconstituted lyophilizate comprises 1.5 mg/mL to 3.7 mg/mL PEG-ADM.

In one embodiment, the reconstituted lyophilizate comprises 1.5 mg/mL to 3.6 mg/mL PEG-ADM.

In one embodiment, the reconstituted lyophilizate comprises 1.5 mg/mL to 3.5 mg/mL PEG-ADM.

In one embodiment, the reconstituted lyophilizate comprises 1.5 mg/mL to 3.4 mg/mL PEG-ADM.

In one embodiment, the reconstituted lyophilizate comprises 1.5 mg/mL to 3.3 mg/mL PEG-ADM.

In one embodiment, the reconstituted lyophilizate comprises 1.5 mg/mL to 3.2 mg/mL PEG-ADM.

In one embodiment, the reconstituted lyophilizate comprises 1.5 mg/mL to 3.1 mg/mL PEG-ADM.

In one embodiment, the reconstituted lyophilizate comprises 1.5 mg/mL to 3 mg/mL PEG-ADM.

In one embodiment, the reconstituted lyophilizate comprises 1.5 mg/mL to 2.9 mg/mL PEG-ADM.

In one embodiment, the reconstituted lyophilizate comprises 1.5 mg/mL to 2.8 mg/mL PEG-ADM.

In one embodiment, the reconstituted lyophilizate comprises 1.5 mg/mL to 2.7 mg/mL PEG-ADM.

In one embodiment, the reconstituted lyophilizate comprises 1.5 mg/mL to 2.6 mg/mL PEG-ADM.

In one embodiment, the reconstituted lyophilizate comprises 1.5 mg/mL to 2.5 mg/mL PEG-ADM.

In one embodiment, the reconstituted lyophilizate comprises 1.5 mg/mL to 2.4 mg/mL PEG-ADM.

In one embodiment, the reconstituted lyophilizate comprises 1.5 mg/mL to 2.3 mg/mL PEG-ADM.

In one embodiment, the reconstituted lyophilizate comprises 1.5 mg/mL to 2.2 mg/mL PEG-ADM.

In one embodiment, the reconstituted lyophilizate comprises 1.5 mg/mL to 2.1 mg/mL PEG-ADM.

In one embodiment, the reconstituted lyophilizate comprises 1.5 mg/mL to 2 mg/mL PEG-ADM.

In one embodiment, the reconstituted lyophilizate comprises 1.5 mg/mL to 1.9 mg/mL PEG-ADM.

In one embodiment, the reconstituted lyophilizate comprises 0.01 mg/mL to 1.9 mg/mL PEG-ADM.

In one embodiment, the reconstituted lyophilizate comprises 1.5 mg/mL to 3.7 mg/mL PEG-ADM.

In one embodiment, the reconstituted lyophilizate comprises 1.5 mg/mL to 3.6 mg/mL PEG-ADM.

In one embodiment, the reconstituted lyophilizate comprises 1.5 mg/mL to 3.5 mg/mL PEG-ADM.

In one embodiment, the reconstituted lyophilizate comprises 1.5 mg/mL to 3.4 mg/mL PEG-ADM.

In one embodiment, the reconstituted lyophilizate comprises 1.5 mg/mL to 3.3 mg/mL PEG-ADM.

In one embodiment, the reconstituted lyophilizate comprises 1.5 mg/mL to 3.2 mg/mL PEG-ADM.

In one embodiment, the reconstituted lyophilizate comprises 1.5 mg/mL to 3.1 mg/mL PEG-ADM.

In one embodiment, the reconstituted lyophilizate comprises 1.5 mg/mL to 3 mg/mL PEG-ADM.

In one embodiment, the reconstituted lyophilizate comprises 1.5 mg/mL to 2.9 mg/mL PEG-ADM.

In one embodiment, the reconstituted lyophilizate comprises 1.5 mg/mL to 2.8 mg/mL PEG-ADM.

In one embodiment, the reconstituted lyophilizate comprises 1.5 mg/mL to 2.7 mg/mL PEG-ADM.

In one embodiment, the reconstituted lyophilizate comprises 1.5 mg/mL to 2.6 mg/mL PEG-ADM.

In one embodiment, the reconstituted lyophilizate comprises 1.5 mg/mL to 2.5 mg/mL PEG-ADM.

In one embodiment, the reconstituted lyophilizate comprises 1.5 mg/mL to 2.4 mg/mL PEG-ADM.

In one embodiment, the reconstituted lyophilizate comprises 1.5 mg/mL to 2.3 mg/mL PEG-ADM.

In one embodiment, the reconstituted lyophilizate comprises 1.5 mg/mL to 2.2 mg/mL PEG-ADM.

In one embodiment, the reconstituted lyophilizate comprises 1.5 mg/mL to 2.1 mg/mL PEG-ADM.

In one embodiment, the reconstituted lyophilizate comprises 1.5 mg/mL to 2 mg/mL PEG-ADM.

In one embodiment, the reconstituted lyophilizate comprises 1.5 mg/mL to 1.9 mg/mL PEG-ADM.

In one embodiment, the reconstituted lyophilizate comprises 1.5 mg/mL to 1.9 mg/mL PEG-ADM.

In one embodiment, the reconstituted lyophilizate comprises 1 mg/mL to 300 mg/mL trehalose.

In one embodiment, the reconstituted lyophilizate comprises 0.1 mg/mL to 200 mg/mL trehalose.

In one embodiment, the reconstituted lyophilizate comprises 10 mg/mL to 100 mg/mL trehalose.

In one embodiment, the reconstituted lyophilizate comprises 30 mg/mL to 70 mg/mL trehalose.

In one embodiment, the reconstituted lyophilizate comprises 40 mg/mL to 60 mg/mL trehalose.

In one embodiment, the reconstituted lyophilizate comprises 0.3 mg/mL to 30 mg/mL of a pH regulator.

In one embodiment, the reconstituted lyophilizate comprises 1 mg/mL to 15 mg/mL of a pH regulator.

In one embodiment, the reconstituted lyophilizate comprises 2 mg/mL to 10 mg/mL of a pH regulator.

In one embodiment, the reconstituted lyophilizate comprises 4 mg/mL to 7 mg/mL of a pH regulator.

In one embodiment, the reconstituted lyophilizate comprises 0.1 mg/mL to 100 mg/mL citric acid.

In one embodiment, the reconstituted lyophilizate comprises 0.3 mg/mL to 30 mg/mL citric acid.

In one embodiment, the reconstituted lyophilizate comprises 1 mg/mL to 15 mg/mL citric acid.

In one embodiment, the reconstituted lyophilizate comprises 2 mg/mL to 10 mg/mL citric acid.

In one embodiment, the reconstituted lyophilizate comprises 4 mg/mL to 7 mg/mL citric acid.

### V. Excipients

The pharmaceutical formulation according to the invention or any embodiment disclosed herein can further comprise at least one excipient. In the context of the present invention, excipients are substances which, in the pharmaceutical formulation serve the purpose, for example, of microbiologically, chemically and physically stabilizing the preparation or improving the taste or optical appearance. The term “excipients” also comprises with an inert nontoxic pharmaceutically suitable excipient. Examples of excipients in the context of the present invention are antioxidants, stabilizers, preservatives, substances for adjusting tonicity, aromas, fragrances or dyes.

### VI. Combined Pharmaceutical Dosage Form

The invention also provides a combined pharmaceutical dose form comprising the pharmaceutical formulation according to any one of the embodiments disclosed in section I to V.

In one embodiment according to the invention, the combination is a combined pharmaceutical dose form. The “combined pharmaceutical dose form” is used to combine two or more pharmaceutical dose forms into a single term, in order to describe a medicinal product that consists of two or more manufactured items that are intended to be combined to produce a single pharmaceutical product for administration to the patient. A combined pharmaceutical dose form is not used to combine pharmaceutical dose forms that are packaged together but administered separately rather than being combined to produce a single pharmaceutical product (see instead combination packs). “Pharmaceutical dose form” and “dosage form” are synonyms. “Pharmaceutical dose form” or “dosage form” is the physical manifestation of a product that contains or comprises the active ingredient and/or inactive ingredients (e.g. carrier, excipients) that are intended to be delivered to the patient. “Dosage form” is the term used in the European Pharmacopoeia. “Dosage form” was previously used in Standard Terms, but the term “pharmaceutical dose form” is now used in order to harmonize with the vocabulary that is used across the Identification of Medicinal Products project (cf. https://www.edqm.eu/sites/default/files/standard_terms_introduction_and_guidance_for_use.pdf). Common dosage forms include pill, tablet, capsule, syrup, aerosol, liquid injection, powder, or solid crystal, and so on. Further pharmaceutical formulations or dosage forms are disclosed below. The route of administration for drug delivery is dependent on the dosage form of the active ingredient.

### VII. Combination Pack

The invention also provides a combination pack comprising the pharmaceutical formulation according to any one of the embodiments disclosed in section I to VI.

One aspect of the present invention is a combination pack. In a “combination pack” the components are included in separate dosage forms marketed in the same package. A combination is different from a combined pharmaceutical dose form. In one embodiment, the combination pack comprises any one of the embodiments of the pharmaceutical formulation disclosed herein and a nebulizer. In one embodiment the nebulizer is a mesh nebulizer or vibrating mesh nebulizer. In one embodiment the nebulizer is an Aerogen Solo nebulizer optionally combined with a Aerogen® Pro-X or Aerogen® USB controller.

### VIII. Indications

The invention further provides:

The pharmaceutical formulation according to any one of the embodiments disclosed in sections I to VII for use in the treatment and/or prevention of diseases.

The use of the pharmaceutical formulation according to any one of the embodiments disclosed in sections I to VII for the treatment and/or prevention of a disease and/or disorder.

The pharmaceutical formulation according to any one of the embodiments disclosed in sections I to VII for producing a medicament for treatment and/or prevention of a disease and/or disorder.

A method of treatment and/or prevention of a disorder and/or disease comprising administering the pharmaceutical formulation according to any one of the embodiments disclosed in sections I to VII.

In this section VIII, “pharmaceutical formulation” or “formulation” or “liquid pharmaceutical formulation” refers to any one of the embodiments disclosed in sections I to VII.

In one embodiment the pharmaceutical formulation according to the invention and the compounds according to formula (I) or (Ia) are suitable for treatment and/or prevention of pulmonary disorders, such as pulmonary hypertension; secondary pulmonary hypertension; pulmonary hypertension following pulmonary embolism with and without acute cor pulmonale; primary pulmonary hypertension; chronic obstructive pulmonary disease; asthma; acute pulmonary edema; chronic pulmonary edema; allergic alveolitis; pneumonitis due to inhaled organic dust; pneumonitis due to inhaled particles of fungal, actinomycetic or other origin; acute chemical bronchitis; acute chemical pulmonary edema and/or chronic chemical pulmonary edema (e.g. after inhalation of phosgene, nitrogen oxide); neurogenic pulmonary edema; acute pulmonary manifestations due to radiation; chronic pulmonary manifestations due to radiation; acute and/or chronic interstitial lung disorders (such as but not restricted to drug-induced interstitial lung disorders, e.g. secondary to Bleomycin treatment); acute lung injury (ALI); acute lung injury (ALI) in adult or child including newborn; acute respiratory distress syndrome (ARDS); acute respiratory distress syndrome (ARDS) in adult or child including newborn; ALI/ARDS secondary to pneumonia and sepsis, aspiration pneumonia and ALI/ARDS secondary to aspiration (such as but not restricted to aspiration pneumonia due to regurgitated gastric content); ALI/ARDS secondary to smoke gas inhalation; transfusion-related acute lung injury (TRALI), ALI/ARDS or acute pulmonary insufficiency following surgery; trauma or burns, ventilator induced lung injury (VILI); lung injury following meconium aspiration; pulmonary fibrosis; and mountain sickness.

In one embodiment the pharmaceutical formulation according to the invention and the compounds according to formula (I) or (Ia) are suitable for treatment and/or prevention of ALI/ARDS secondary to pneumonia caused by bacterial infection of the lungs, such as, but not restricted to, bacterial pneumonia caused by Pneumococci, Haemophilus Influenzae, Mycoplasma Pneumoniae, Chlamydia species, Enterococci, beta-hemolytic Streptococci, Staphylococci, Gram-negative Enterobacteriaceae, Pseudomonas species, Klebsiella species, Acinetobacter species, Legionella species, and Mycobacteria.

In one embodiment the pharmaceutical formulation according to the invention and the compounds according to formula (I) or (Ia) are suitable for treatment and/or prevention of ALI/ARDS secondary to pneumonia caused by viral infections such as, but not restricted to, Influenza viruses (e.g. caused by strains of serotypes H1N1, H5N1, H7N9), Corona viruses (e.g. SARS-CoV, the pathogen of severe acute respiratory syndrome (SARS), MERS-CoV, the pathogen of Middle East respiratory syndrome (MERS), and SARS-CoV-2 the pathogen of COVID-19 pandemic), Respiratory-Syncytial-Virus (RSV), and Cytomegalovirus (CMV).

In one embodiment the pharmaceutical formulation according to the invention and the compounds according to formula (I) or (Ia) are also suitable for treatment and/or prevention of ALI/ARDS secondary to pneumonia caused by fungal infections such as, but not restricted to, fungal pneumonia caused by Pneumocystis Jirovecii.

In one embodiment the pharmaceutical formulation according to the invention and the compounds according to formula (I) or (Ia) are suitable for treatment and/or prevention of ALI/ARDS secondary to pneumonia irrespective of the context of pneumonia origin such as for community acquired pneumonia (CAP) as well as for hospital acquired pneumonia (HAP), in particular for HAP acquired in the context of artificial ventilation (VAP).

In one embodiment the pharmaceutical formulation according to the invention and the compounds according to formula (I) or (Ia) are suitable for treatment and/or prevention of ALI/ARDS secondary to pneumonia irrespective of the diverse pathoanatomical appearances of pneumonias such as, but not restricted to, lobar (i.e. affecting an entire lung lobe), lobular (i.e. affecting smaller lung lobules), interstitial (i.e. diffuse affection of the lung tissue).

In one embodiment the pharmaceutical formulation according to the invention and the compounds according to formula (I) or (Ia) are suitable for treatment and/or prevention of ALI/ARDS secondary to pneumonia occurring in consequence of bacterial and/or virus infection.

In one embodiment the pharmaceutical formulation according to the invention and the compounds according to formula (I) or (Ia) are suitable for treatment and/or prevention of ALI/ARDS secondary to pneumonia occurring in consequence of a bacterial superinfection of a primary lung affection by viruses.

In one embodiment the pharmaceutical formulation according to the invention and the compounds according to formula (I) or (Ia) are suited for the prevention and/or treatment of lung dysfunction after lung transplantations.

On the basis of their pharmacological properties, the pharmaceutical formulation according to the invention and the compounds according to formula (I) or (Ia) according to the invention can be employed to prevent and /or ameliorate development of sepsis secondary to bacterial pneumonia (so called pneumogenic sepsis).

A further embodiment is the compound according to formula (I) or the compound according to formula (Ia) for use in the treatment and/or prevention of the disorders and/or diseases listed in this section “Indications”. The pharmaceutical formulation according to the invention and the compounds according to formula (I) or (Ia) are in particular suitable for treatment and/or prevention of ALI/ARDS in immunocompromised patients suffering from pneumonia, such as in the context of acquired immunodeficiency syndrome (AIDS), chemotherapy and bone marrow transplantation.

### IX. Product by Process

The invention further provides:

A lyophilizate according to any one of the embodiments disclosed in section II obtainable by freeze-drying of the liquid pharmaceutical formulation according to any one of the embodiments disclosed in section III.

Liquid Pharmaceutical formulation according to any one of the embodiments disclosed in section III. obtainable by mixing the lyophilizate according to any one of the embodiments disclosed in section II with a solvent.

The invention also provides a pharmaceutical formulation according as described in any one of the embodiments in section II obtainable by the method according to any one of the embodiments disclosed in section II.vi.

The invention also provides the liquid pharmaceutical formulation obtainable by the method described in section III.x.

### X. Clauses

The following clauses disclosed further embodiments according to the invention:

1. A pharmaceutical formulation comprising:
PEG-ADM, wherein the PEG-ADM is a compound according to the general formula (I),

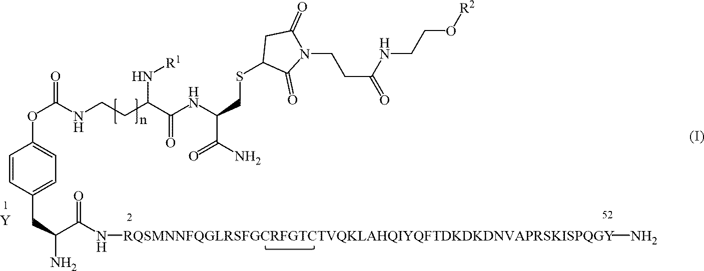

in which
   n represents the number 0, 1, 2 or 3,
   R¹ represents hydrogen, methyl, ethyl, n-propyl or isopropyl,
   R² represents linear or branched PEG 20 kDa to 80 kDa endcapped with a methoxy-group,
   or a hydrate thereof, solvate thereof, salt thereof, pharmaceutically acceptable salt thereof,
   or the solvates of salts thereof;
a pH regulator; and
trehalose or a hydrate thereof, solvate thereof, salt thereof, pharmaceutically acceptable salt thereof, or the solvates of salts thereof;
wherein the concentrations of components are based on the total weight of the pharmaceutical formulation.

2. The pharmaceutical formulation according to clause 1, wherein comprises
1 wt.-% to 15 wt.% of PEG-ADM or a hydrate thereof, solvate thereof, salt thereof, pharmaceutically acceptable salt thereof, or the solvates of salts thereof;
0.01 wt.-% to 25 wt.% of the pH regulator; and
60 wt.-% to 98 wt.-% trehalose or a hydrate thereof, solvate thereof, salt thereof, pharmaceutically acceptable salt thereof, or the solvates of salts thereof;
wherein the concentrations of components are based on the total weight of the pharmaceutical formulation.

3. The pharmaceutical formulation according to any one of the preceding clauses, wherein the pharmaceutical formulation is a lyophilizate.

4. The pharmaceutical formulation according to any one of the preceding clauses, wherein the lyophilizate comprises 3 wt.-% to 10 wt.-% PEG-ADM as defined in any one of the preceding clauses, wherein the concentration is based on the total weight of the pharmaceutical formulation.

5. The pharmaceutical formulation according to any one of the preceding clauses, wherein the pH regulator is a buffer.

6. The pharmaceutical formulation according to any one of the preceding clauses, wherein the pH regulator is selected from the group consisting of citrate, citric acid, a salt of citric acid, a pharmaceutical acceptable salt of citric acid, a derivative of citric acid, and/or mixtures thereof.

7. The pharmaceutical formulation according to any one of the preceding clauses, wherein the lyophilizate comprises 3 wt.-% to 12 wt.-% of a pH regulator as defined in any one of the preceding clauses, wherein the concentration is based on the total weight of the pharmaceutical formulation.

8. The pharmaceutical formulation according to any one of the preceding clauses, wherein trehalose is selected from the group of trehalose dihydrate, trehalose anhydrate and/or mixtures thereof.

9. The pharmaceutical formulation according to any one of the preceding clauses, wherein the lyophilizate comprises 70 wt.-% to 85 wt.-% of trehalose as defined in any one of the preceding clauses, wherein the concentration is based on the total weight of the pharmaceutical formulation.

10. A liquid pharmaceutical formulation comprising:
a. 0.04 mg/mL to 145 mg/mL of PEG-ADM, wherein the PEG-ADM is a compound according to the general formula (I),

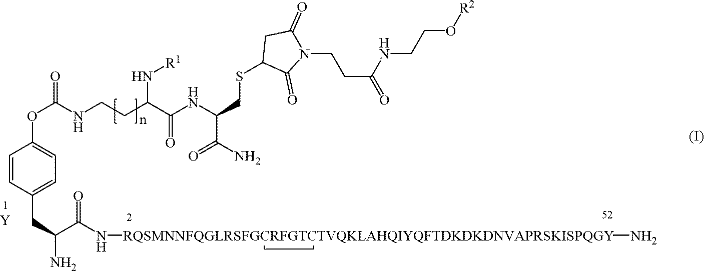

in which
   n represents the number 0, 1, 2 or 3,
   R¹ represents hydrogen, methyl, ethyl, n-propyl or isopropyl,
   R² represents linear or branched PEG 20 kDa to 80 kDa endcapped with a methoxy-group,
      or a hydrate thereof, solvate thereof, salt thereof, pharmaceutically acceptable salt thereof,
      or the solvates of salts thereof;
b. a solvent;
c. a pH regulator;
d. an osmolarity regulator; and
e. trehalose;
   wherein the presence of the osmolarity regulator (component d) is optional;
   wherein the pharmaceutical formulation has a pH between 3 and 5; and
   wherein the concentrations of components are based on the total volume of the liquid pharmaceutical formulation.

11. The pharmaceutical formulation according to clause 3, wherein the pharmaceutical formulation is a solution, an aqueous solution or dispersion.

12. The pharmaceutical formulation according to any one of the preceding clauses, wherein the solvent is a solvent selected from water, sodium chloride solution, buffer solution and mixtures thereof.

13. The pharmaceutical formulation according to any one of the preceding clauses, wherein the solvent comprises water.

14. The pharmaceutical formulation according to any one of the preceding clauses, wherein the solvent is water.

15. The pharmaceutical formulation according to any one of the preceding clauses, wherein the solvent comprises a sodium chloride solution.

16. The pharmaceutical formulation according to any one of the preceding clauses, wherein the solvent is a sodium chloride solution.

17. The pharmaceutical formulation according to any one of the preceding clauses, wherein the solvent is an isotonic sodium chloride solution.

18. The pharmaceutical formulation according to any one of the preceding clauses, wherein the solvent comprises a buffer.

19. The pharmaceutical formulation according to any one of the preceding clauses, wherein the solvent is a buffer.

20. The pharmaceutical formulation according to any one of the preceding clauses, wherein the buffer is selected from citrate buffer (pH 3-6.2; pKa 3.3/4.8/6.4), phosphate citrate buffer (pH 2.2-8.0, pKa = 7.2/6.4/2.2), phosphate buffer (pH 2-12; pKa 2.2/6.9/12.3), sodium acetate buffer (pH 3.6-5.6, pKa 4.76), glycine-HCl (pH 2.2-3.6, pKa 2.35), leucine buffer (pH 2-4; pKa 2.3), aspartic acid buffer (pH 3-5; pKa 2.0/3.9), glutamic acid buffer (pH 3-6; pKa 2.2/4.3).

21. The pharmaceutical formulation according to any one of the preceding clauses, wherein the solvent is selected from the group of water, sodium chloride solution, solution of citric acid, solution of citric acid anhydrous, solution of citric acid monohydrate, hydrochloric acid, sodium hydroxide solution, sodium citrate solution, and/or mixtures.

22. The pharmaceutical formulation according to any one of the preceding clauses, wherein the solvent is water; or comprises a mixture of water and sodium chloride; or comprises a mixture of water and sodium citrate.

23. The pharmaceutical formulation according to any one of the preceding clauses, wherein the PEG-ADM is selected from compounds of the general formula (I) and R² represents linear or branched PEG 20 kDa endcapped with a methoxy-group, wherein the PEG-ADM is a compound according to the general formula (I) as defined in any one of the preceding clauses, or a hydrate thereof, solvate thereof, salt thereof, pharmaceutically acceptable salt thereof, or the solvates of salts thereof.

24. The pharmaceutical formulation according to any one of the preceding clauses, wherein the PEG-ADM is selected from compounds of the general formula (I) and R² represents linear or branched PEG 40 kDa endcapped with a methoxy-group, wherein the PEG-ADM is a compound according to the general formula (I) as defined in any one of the preceding clauses, or a hydrate thereof, solvate thereof, salt thereof, pharmaceutically acceptable salt thereof, or the solvates of salts thereof.

25. The pharmaceutical formulation according to any one of the preceding clauses, wherein the PEG-ADM is selected from compounds of the general formula (I) and R² represents linear or branched PEG 80 kDa endcapped with a methoxy-group, wherein the PEG-ADM is a compound according to the general formula (I) as defined in any one of the preceding clauses, or a hydrate thereof, solvate thereof, salt thereof, pharmaceutically acceptable salt thereof, or the solvates of salts thereof.

26. The pharmaceutical formulation according to any one of the preceding clauses, wherein the PEG-ADM is selected from compounds of the general formula (I),

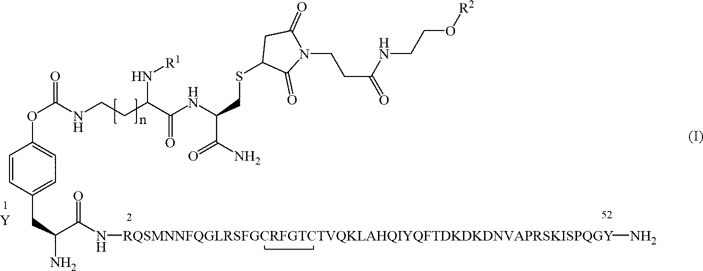

in which
n represents the number 0, 1, 2 or 3,
R¹ represents hydrogen, methyl, ethyl, n-propyl or isopropyl,
R² represents linear or branched PEG 20 kDa to 80 kDa endcapped with a methoxy-group,
or a hydrate thereof, solvate thereof, salt thereof, pharmaceutically acceptable salt thereof, or the solvates of salts thereof.

27. The pharmaceutical formulations according to any one of the preceding clauses, wherein the PEG-ADM is selected from compounds of the formula (I) in which
n represents the number 1 or 2,
R¹ represents hydrogen or methyl,
R² represents linear PEG 40 kDa endcapped with a methoxy-group.

28. The pharmaceutical formulations according to any one of the preceding clauses, wherein the PEG-ADM is selected from compounds of the formula (I) in which
n represents the number 1 or 2,
R¹ represents hydrogen,
R² represents linear PEG 40 kDa endcapped with a methoxy-group.

29. The pharmaceutical formulation according to any one of the preceding clauses, wherein the PEG-ADM is the compound according to formula (Ia)

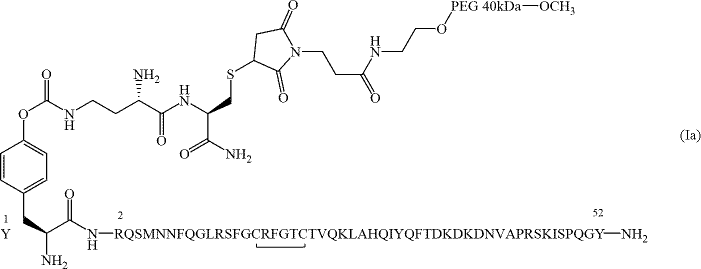

30. The pharmaceutical formulation according to any one of the preceding clauses, wherein the pharmaceutical formulation comprises 0.385 mg/mL to 77 mg/mL PEG-ADM.

31. The pharmaceutical formulation according to any one of the preceding clauses, wherein the pharmaceutical formulation comprises 2.31 mg/mL to 77 mg/mL PEG-ADM.

32. The pharmaceutical formulation according to any one of the preceding clauses, wherein the pharmaceutical formulation comprises 3.85 mg/mL to 77 mg/mL PEG-ADM.

33. The pharmaceutical formulation according to any one of the preceding clauses, wherein, wherein the pharmaceutical formulation comprises 7.7 mg/mL to 77 mg/mL PEG-ADM.

34. The pharmaceutical formulation according to any one of the preceding clauses, wherein the pharmaceutical formulation comprises 0.385 mg/mL to 38.5 mg/mL PEG-ADM.

35. The pharmaceutical formulation according to any one of the preceding clauses, wherein the pharmaceutical formulation comprises 0.77 mg/mL to 38.5 mg/mL PEGADM.

36. The pharmaceutical formulation according to any one of the preceding clauses, wherein the pharmaceutical formulation comprises 0.77 mg/mL to 23.1 mg/mL PEGADM.

37. The pharmaceutical formulation according to any one of the preceding clauses, wherein the pharmaceutical formulation comprises 0.77 mg/mL to 7.7 mg/mL PEG-ADM.

38. The pharmaceutical formulation according to any one of the preceding clauses, wherein the pharmaceutical formulation comprises 2.31 mg/mL to 7.7 mg/mL PEG-ADM.

39. The pharmaceutical formulation according to any one of the preceding clauses, wherein the pharmaceutical formulation comprises 2.31 mg/mL to 3.85 mg/mL PEG-ADM.

40. The pharmaceutical formulation according to any one of the preceding clauses, wherein the pharmaceutical formulation comprises 3.08 mg/mL to 23.1 mg/mL PEG-ADM.

41. The pharmaceutical formulation according to any one of the preceding clauses, wherein the pharmaceutical formulation comprises 3.08 mg/mL to 7.7 mg/mL PEG-ADM.

42. The pharmaceutical formulation according to any one of the preceding clauses, wherein the pharmaceutical formulation comprises 0.77 mg/mL PEG-ADM.

43. The pharmaceutical formulation according to any one of the preceding clauses, wherein the pharmaceutical formulation comprises 6.16 mg/mL PEG-ADM.

44. The pharmaceutical formulation according to any one of the preceding clauses, wherein the pharmaceutical formulation comprises 4.6 g/mL PEG-ADM.

45. The pharmaceutical formulation according to any one of the preceding clauses, wherein the pharmaceutical formulation comprises 3.85 mg/mL PEG-ADM.

46. The pharmaceutical formulation according to any one of the preceding clauses, wherein the pharmaceutical formulation comprises 3.7 mg/mL PEG-ADM.

47. The pharmaceutical formulation according to any one of the preceding clauses, wherein the pharmaceutical formulation comprises 2.31 mg/mL PEG-ADM.

48. The pharmaceutical formulation according to any one of the preceding clauses, wherein the pharmaceutical formulation comprises approximately 0.044 mg/mL to 44 mg/mL PEG-ADM.

49. The pharmaceutical formulation according to any one of the preceding clauses, wherein the pharmaceutical formulation comprises approximately 0.22 mg/mL to 22 mg/mL PEG-ADM.

50. The pharmaceutical formulation according to any one of the preceding clauses, wherein the pharmaceutical formulation comprises approximately 0.44 mg/mL to 13.2 mg/mL PEG-ADM.

51. The pharmaceutical formulation according to any one of the preceding clauses, wherein the pharmaceutical formulation comprises approximately 0.44 mg/mL to 4.4 mg/mL PEG-ADM.

52. The pharmaceutical formulation according to any one of the preceding clauses, wherein the pharmaceutical formulation comprises approximately 1.3 mg/mL to 2.2 mg/mL PEG-ADM.

53. The pharmaceutical formulation according to any one of the preceding clauses, wherein the pharmaceutical formulation comprises approximately 0.14 mg/mL to 144 mg/mL PEG-ADM.

54. The pharmaceutical formulation according to any one of the preceding clauses, wherein the pharmaceutical formulation comprises approximately 0.7 mg/mL to 71.7 mg/mL PEG-ADM.

55. The pharmaceutical formulation according to any one of the preceding clauses, wherein the pharmaceutical formulation comprises approximately 1.4 mg/mL to 43 mg/mL PEG-ADM.

56. The pharmaceutical formulation according to any one of the preceding clauses, wherein the pharmaceutical formulation comprises approximately 1.4 mg/mL to 14.3 mg/mL PEG-ADM.

57. The pharmaceutical formulation according to any one of the preceding clauses, wherein the pharmaceutical formulation comprises approximately 4.3 mg/mL to 7.2 mg/mL PEG-ADM.

58. The pharmaceutical formulation according to any one of the preceding clauses, wherein the stabilizer trehalose is selected from the group of trehalose dihydrate,trehalose anhydrate and/or mixtures.

59. The pharmaceutical formulation according to any one of the preceding clauses, wherein the pharmaceutical formulation comprises 1 mg/mL to 300 mg/mL of trehalose.

60. The pharmaceutical formulation according to any one of the preceding clauses, wherein the pharmaceutical formulation comprises 5 mg/mL to 200 mg/mL of trehalose.

61. The pharmaceutical formulation according to any one of the preceding clauses, wherein the pharmaceutical formulation comprises 10 mg/mL to 100 mg/mL of trehalose.

62. The pharmaceutical formulation according to any one of the preceding clauses, wherein the pharmaceutical formulation comprises 30 mg/mL to 70 mg/mL of trehalose.

63. The pharmaceutical formulation according to any one of the preceding clauses, wherein the pharmaceutical formulation comprises 40 mg/mL to 60 mg/mL of trehalose.

64. The pharmaceutical formulation according to any one of the preceding clauses, wherein the pharmaceutical formulation comprises 0.1 mg/mL to 250 mg/mL of the pH regulator.

65. The pharmaceutical formulation according to any one of the preceding clauses, wherein the pharmaceutical formulation comprises 0.3 mg/mL to 250 mg/mL of the pH regulator.

66. The pharmaceutical formulation according to any one of the preceding clauses, wherein the pharmaceutical formulation comprises 0.5 mg/mL to 100 mg/mL of the pH regulator.

67. The pharmaceutical formulation according to any one of the preceding clauses, wherein the pharmaceutical formulation comprises 0.9 mg/mL to 90 mg/mL of the pH regulator.

68. The pharmaceutical formulation according to any one of the preceding clauses, wherein the pharmaceutical formulation comprises 2.5 mg/mL to 46 mg/mL of the pH regulator.

69. The pharmaceutical formulation according to any one of the preceding clauses, wherein the pharmaceutical formulation comprises 7.8 mg/mL to 29 mg/mL of the pH regulator.

70. The pharmaceutical formulation according to any one of the preceding clauses, wherein the pharmaceutical formulation comprises 12.5 mg/mL to 19 mg/mL of the pH regulator.

71. The pharmaceutical formulation according to any one of the preceding clauses, wherein the pharmaceutical formulation comprises 0.01 mg/mL to 100 mg/mL of the pH regulator.

72. The pharmaceutical formulation according to any one of the preceding clauses, wherein the pharmaceutical formulation comprises 0.1 mg/mL to 50 mg/mL of the pH regulator.

73. The pharmaceutical formulation according to any one of the preceding clauses, wherein the pharmaceutical formulation comprises 0.5 mg/mL to 25 mg/mL of the pH regulator.

74. The pharmaceutical formulation according to any one of the preceding clauses, wherein the pharmaceutical formulation comprises 0.8 mg/mL to 15 mg/mL of the pH regulator.

75. The pharmaceutical formulation according to any one of the preceding clauses, wherein the pharmaceutical formulation comprises 1.5 mg/mL to 9 mg/mL of the pH regulator.

76. The pharmaceutical formulation according to any one of the preceding clauses, wherein the pH regulator comprises citric acid, a salt of citric acid, a pharmaceutical acceptable salt of citric acid, a derivative of citric acid, and/or mixtures thereof.

77. The pharmaceutical formulation according to any one of the preceding clauses, wherein the pH regulator comprises hydrochloric acid, citric acid, a salt of citric acid, pharmaceutical acceptable salt of citric acid, derivative of citric acid, and/or mixtures thereof.

78. The pharmaceutical formulation according to any one of the preceding clauses, wherein the pH regulator comprises hydrochloric acid.

79. The pharmaceutical formulation according to any one of the preceding clauses, wherein the pH regulator comprises a mixture comprising hydrochloric acid and sodium hydroxide.

80. The pharmaceutical formulation according to any one of the preceding clauses, wherein the pH regulator comprises a mixture comprising hydrochloric acid, sodium hydroxide and citric acid.

81. The pharmaceutical formulation according to any one of the preceding clauses, wherein the pH regulator comprises a mixture comprising sodium hydroxide and citric acid.

82. The pharmaceutical formulation according to any one of the preceding clauses, wherein the pH regulator comprises a mixture comprising sodium citrate and hydrochloric acid.

83. The pharmaceutical formulation according to any one of the preceding clauses, wherein the pH regulator consists of hydrochloric acid.

84. The pharmaceutical formulation according to any one of the preceding clauses, wherein the pH regulator consists of a mixture comprising hydrochloric acid and sodium hydroxide.

85. The pharmaceutical formulation according to any one of the preceding clauses, wherein the pH regulator consists of a mixture comprising hydrochloric acid, sodium hydroxide and citric acid.

86. The pharmaceutical formulation according to any one of the preceding clauses, wherein the pH regulator consists of a mixture comprising sodium hydroxide and citric acid.

87. The pharmaceutical formulation according to any one of the preceding clauses, wherein the pH regulator consists of a mixture comprising sodium citrate and hydrochloric acid.

88. The pharmaceutical formulation according to any one of the preceding clauses, wherein the citric acid is a salt of citric acid, pharmaceutical acceptable salt of citric acid, derivative of citric acid, and/or mixtures thereof.

89. The pharmaceutical formulation according to any one of the preceding clauses, wherein the salt of citric acid, pharmaceutical acceptable salt of citric acid, derivative of citric acid, and/or mixtures thereof is selected from the group consisting of citric acid anhydrous, sodium citrate and citric acid monohydrate.

90. The pharmaceutical formulation according to any one of the preceding clauses, wherein the pH regulator comprises or consists of hydrochloric acid, preferably hydrochloric acid

91. The pharmaceutical formulation according to any one of the preceding clauses, wherein the pH regulator comprises or consists of 0.1 mg/mL to 100 mg/mL citric acid.

92. The pharmaceutical formulation according to any one of the preceding clauses, wherein the pH regulator comprises or consists of 0.3 mg/mL to 30 mg/mL citric acid.

93. The pharmaceutical formulation according to any one of the preceding clauses, wherein the pH regulator comprises or consists of 1 mg/mL to 15 mg/mL citric acid.

94. The pharmaceutical formulation according to any one of the preceding clauses, wherein the pH regulator comprises or consists of 2 mg/mL to 10 mg/mL citric acid.

95. The pharmaceutical formulation according to any one of the preceding clauses, wherein the pH regulator comprises or consists of 4 mg/mL to 7 mg/mL citric acid.

96. The pharmaceutical formulation according to any one of the preceding clauses, wherein the pH regulator comprises or consists of wherein the pH regulator comprises or consists of 0.01 mg/mL to 50 mg/mL sodium hydroxide.

97. The pharmaceutical formulation according to any one of the preceding clauses, wherein the pH regulator comprises or consists of 0.1 mg/mL to 10 mg/mL sodium hydroxide.

98. The pharmaceutical formulation according to any one of the preceding clauses, wherein the pH regulator comprises or consists of 0.5 mg/mL to 6 mg/mL sodium hydroxide.

99. The pharmaceutical formulation according to any one of the preceding clauses, wherein the pH regulator comprises or consists of 0.8 mg/mL to 4 mg/mL sodium hydroxide.

100. The pharmaceutical formulation according to any one of the preceding clauses, wherein the pH regulator comprises or consists of 0.1 mg/mL to 100 mg/mL hydrochloric acid.

101. The pharmaceutical formulation according to any one of the preceding clauses, wherein the pH regulator comprises or consists of 0.5 mg/mL to 50 mg/mL hydrochloric acid.

102. The pharmaceutical formulation according to any one of the preceding clauses, wherein the pH regulator comprises or consists of 1 mg/mL to 25 mg/mL hydrochloric acid.

103. The pharmaceutical formulation according to any one of the preceding clauses, wherein the pH regulator comprises or consists of 5 mg/mL to 15 mg/mL of hydrochloric acid 10% (m/V).

104. The pharmaceutical formulation according to any one of the preceding clauses, wherein the pharmaceutical formulation comprises two or more pH regulators.

105. The pharmaceutical formulation according to any one of the preceding clauses, wherein the pharmaceutical formulation comprises three or more pH regulators.

106. The pharmaceutical formulation according to any one of the preceding clauses, wherein the osmolarity regulator is selected from the group consisting of sodium chloride, citric acid, a salt, pharmaceutical acceptable salt, derivative of citric acid and/or mixtures thereof.

107. The pharmaceutical formulation according to any one of the preceding clauses, wherein the citric acid is a salt, pharmaceutical acceptable salt, derivative of citric acid is selected from the group consisting of citric acid anhydrous, sodium citrate and citric acid monohydrate.

108. The pharmaceutical formulation according to any one of the preceding clauses, wherein the osmolarity regulator is sodium chloride.

109. The pharmaceutical formulation according to any one of the preceding clauses, wherein the pharmaceutical formulation comprises 0.01 mg/mL to 50 mg/mL of the osmolarity regulator.

110. The pharmaceutical formulation according to any one of the preceding clauses, wherein the pharmaceutical formulation comprises 0.1 mg/mL to 30 mg/mL of the osmolarity regulator.

111. The pharmaceutical formulation according to any one of the preceding clauses, wherein the pharmaceutical formulation comprises 0.3 mg/mL to 15 mg/mL of the osmolarity regulator.

112. The pharmaceutical formulation according to any one of the preceding clauses, wherein the pharmaceutical formulation comprises 0.5 mg/mL to 5 mg/mL of the osmolarity regulator.

113. The pharmaceutical formulation according to any one of the preceding clauses, wherein the pharmaceutical formulation comprises 0.7 mg/mL to 2.5 mg/mL of the osmolarity regulator.

114. The pharmaceutical formulation according to any one of the preceding clauses, wherein the pharmaceutical formulation has an osmotic concentration between 150 mosmol/l to 450 mosmol/l or 200 to 400 mosmol/l.

115. The pharmaceutical formulation according to any one of the preceding clauses, wherein the pharmaceutical formulation has an osmotic concentration between 270 to 330 mosmol/l.

116. The pharmaceutical formulation according to any one of the preceding clauses, wherein the pharmaceutical formulation has an osmotic concentration between 250 to 310 mosmol/l.

117. The pharmaceutical formulation according to any one of the preceding clauses, wherein the pharmaceutical formulation has an osmotic concentration of 300 mosmol/l.

118. The pharmaceutical formulation according to any one of the preceding clauses, wherein the formulation has a pH of 3.5 to 4.5.

119. The pharmaceutical formulation according to any one of the preceding clauses, wherein the formulation has a pH of 3 to 4.

120. The pharmaceutical formulation according to any one of the preceding clauses, wherein the formulation has a pH of 3 to 3.5.

121. The pharmaceutical formulation according to any one of the preceding clauses, wherein the formulation has a pH of 3.5 to 4.

122. The pharmaceutical formulation according to any one of the preceding clauses, wherein the formulation has a pH of 3.5.

123. The pharmaceutical formulation according to any one of the preceding clauses, wherein the formulation has a pH of 4.

124. The pharmaceutical formulation according to any one of the preceding clauses, wherein the formulation has a viscosity of 0.9 to 2.2 mPa*s, 1 to 2 mPa*s, 1.05 to 2 mPa*s, 1.1 to 2 mPa*s or 1.05 to 1.9 mPa*s.

125. The pharmaceutical formulation according to any one of the preceding clauses, wherein the pharmaceutical formulation comprises as pH regulator
0.1 mg/mL to 100 mg/mL citric acid;
0.01 mg/mL to 50 mg/mL sodium hydroxide;
0.1 mg/mL to 100 mg/mL hydrochloric acid.

126. The pharmaceutical formulation according to any one of the preceding clauses, wherein the pharmaceutical formulation comprises as pH regulator
0.3 mg/mL to 30 mg/mL citric acid;
0.1 mg/mL to 10 mg/mL sodium hydroxide;
0.5 mg/mL to 50 mg/mL hydrochloric acid.

127. The pharmaceutical formulation according to any one of the preceding clauses, wherein the pharmaceutical formulation comprises as pH regulator
1 mg/mL to 15 mg/mL citric acid;
0.5 mg/mL to 6 mg/mL sodium hydroxide;
1 mg/mL to 25 mg/mL hydrochloric acid.

128. The pharmaceutical formulation according to any one of the preceding clauses, wherein the pharmaceutical formulation comprises as pH regulator
2 mg/mL to 10 mg/mL citric acid;
0.8 mg/mL to 4 mg/mL sodium hydroxide;
5 mg/mL to 15 mg/mL hydrochloric acid.

129. The pharmaceutical formulation according to any one of the preceding clauses, wherein the pharmaceutical formulation comprises
0.077 mg/mL to 77 mg/mL PEG-ADM,
0.1 mg/mL to 100 mg/mL citric acid;
0.01 mg/mL to 50 mg/mL sodium hydroxide;
0.1 mg/mL to 100 mg/mL hydrochloric acid.

130. The pharmaceutical formulation according to any one of the preceding clauses, wherein the pharmaceutical formulation comprises
0.385 mg/mL to 3.85 mg/mL PEG-ADM,
0.3 mg/mL to 30 mg/mL citric acid;
0.1 mg/mL to 10 mg/mL sodium hydroxide;
0.5 mg/mL to 50 mg/mL hydrochloric acid.

131. The pharmaceutical formulation according to any one of the preceding clauses, wherein the pharmaceutical formulation comprises
0.77 mg/mL to 23.1 mg/mL PEG-ADM,
1 mg/mL to 15 mg/mL citric acid;
0.5 mg/mL to 6 mg/mL sodium hydroxide;
1 mg/mL to 25 mg/mL hydrochloric acid.

132. The pharmaceutical formulation according to any one of the preceding clauses, wherein the pharmaceutical formulation comprises
0.77 mg/mL to 7.7 mg/mL PEG-ADM,
2 mg/mL to 10 mg/mL citric acid;
0.8 mg/mL to 4 mg/mL sodium hydroxide; and
5 mg/mL to 15 mg/mL hydrochloric acid.

133. The pharmaceutical formulation according to any one of the preceding clauses, wherein the pharmaceutical formulation comprises
2.31 mg/mL to 3.85 mg/mL PEG-ADM,
4 mg/mL to 7 mg/mL citric acid;
1.5 mg/mL to 3 mg/mL sodium hydroxide; and
7 mg/mL to 9 mg/mL hydrochloric acid.

134. The pharmaceutical formulation according to any one of the preceding clauses, wherein the pharmaceutical formulation comprises
0.077 mg/mL to 77 mg/mL PEG-ADM,
0.1 mg/mL to 100 mg/mL citric acid;
0.01 mg/mL to 50 mg/mL sodium hydroxide;
0.1 mg/mL to 100 mg/mL hydrochloric acid;
0.01 mg/mL to 50 mg/mL sodium chloride.

135. The pharmaceutical formulation according to any one of the preceding clauses, wherein the pharmaceutical formulation comprises
0.385 mg/mL to 3.85 mg/mL PEG-ADM,
0.3 mg/mL to 30 mg/mL citric acid;
0.1 mg/mL to 10 mg/mL sodium hydroxide;
0.5 mg/mL to 50 mg/mL hydrochloric acid;
0.1 mg/mL to 30 mg/mL sodium chloride.

136. The pharmaceutical formulation according to any one of the preceding clauses, wherein the pharmaceutical formulation comprises
0.77 mg/mL to 23.1 mg/mL PEG-ADM,
1 mg/mL to 15 mg/mL citric acid;
0.5 mg/mL to 6 mg/mL sodium hydroxide;
1 mg/mL to 25 mg/mL hydrochloric acid;
0.3 mg/mL to 15 mg/mL sodium chloride.

137. The pharmaceutical formulation according to any one of the preceding clauses, wherein the pharmaceutical formulation comprises
0.77 mg/mL to 7.7 mg/mL PEG-ADM,
2 mg/mL to 10 mg/mL citric acid;
0.8 mg/mL to 4 mg/mL sodium hydroxide;
5 mg/mL to 15 mg/mL hydrochloric acid;
0.5 mg/mL to 5 mg/mL sodium chloride.

138. The pharmaceutical formulation according to any one of the preceding clauses, wherein the pharmaceutical formulation comprises
2.31 mg/mL to 3.85 mg/mL PEG-ADM,
4 mg/mL to 7 mg/mL citric acid;
1.5 mg/mL to 3 mg/mL sodium hydroxide;
7 mg/mL to 9 mg/mL hydrochloric acid;
0.5 mg/mL to 2.5 mg/mL sodium chloride.

139. The pharmaceutical formulation according to any one of the preceding clauses, wherein the pharmaceutical formulation comprises
0.077 mg/mL to 77 mg/mL PEG-ADM,
0.1 mg/mL to 100 mg/mL citric acid;
0.01 mg/mL to 50 mg/mL sodium hydroxide;
0.1 mg/mL to 100 mg/mL hydrochloric acid;
1 mg/mL to 300 mg/mL trehalose.

140. The pharmaceutical formulation according to any one of the preceding clauses, wherein the pharmaceutical formulation comprises
0.385 mg/mL to 3.85 mg/mL PEG-ADM,
0.3 mg/mL to 30 mg/mL citric acid;
0.1 mg/mL to 10 mg/mL sodium hydroxide;
0.5 mg/mL to 50 mg/mL hydrochloric acid;
5 mg/mL to 200 mg/mL trehalose.

141. The pharmaceutical formulation according to any one of the preceding clauses, wherein the pharmaceutical formulation comprises
0.77 mg/mL to 23.1 mg/mL PEG-ADM,
1 mg/mL to 15 mg/mL citric acid;
0.5 mg/mL to 6 mg/mL sodium hydroxide;
1 mg/mL to 25 mg/mL hydrochloric acid;
10 mg/mL to 100 mg/mL trehalose.

142. The pharmaceutical formulation according to any one of the preceding clauses, wherein the pharmaceutical formulation comprises
0.77 mg/mL to 7.7 mg/mL PEG-ADM,
2 mg/mL to 10 mg/mL citric acid;
0.8 mg/mL to 4 mg/mL sodium hydroxide; and
5 mg/mL to 15 mg/mL hydrochloric acid;
0.5 mg/mL to 5 mg/mL trehalose.

143. The pharmaceutical formulation according to any one of the preceding clauses, wherein the pharmaceutical formulation comprises
2.31 mg/mL to 3.85 mg/mL PEG-ADM,
4 mg/mL to 7 mg/mL citric acid;
1.5 mg/mL to 3 mg/mL sodium hydroxide; and
7 mg/mL to 9 mg/mL hydrochloric acid;
40 mg/mL to 60 mg/mL trehalose.

144. The pharmaceutical formulation according to any one of the preceding clauses, wherein the pharmaceutical formulation comprises
0.077 mg/mL to 77 mg/mL PEG-ADM,
0.1 mg/mL to 100 mg/mL citric acid;
0.01 mg/mL to 50 mg/mL sodium hydroxide;
0.1 mg/mL to 100 mg/mL hydrochloric acid;
1 mg/mL to 300 mg/mL trehalose
0.01 mg/mL to 50 mg/mL sodium chloride.

145. The pharmaceutical formulation according to any one of the preceding clauses, wherein the pharmaceutical formulation comprises
0.385 mg/mL to 3.85 mg/mL PEG-ADM,
0.3 mg/mL to 30 mg/mL citric acid;
0.1 mg/mL to 10 mg/mL sodium hydroxide;
0.5 mg/mL to 50 mg/mL hydrochloric acid;
5 mg/mL to 200 mg/mL trehalose
0.1 mg/mL to 30 mg/mL sodium chloride.

146. The pharmaceutical formulation according to any one of the preceding clauses, wherein the pharmaceutical formulation comprises
0.77 mg/mL to 23.1 mg/mL PEG-ADM,
1 mg/mL to 15 mg/mL citric acid;
0.5 mg/mL to 6 mg/mL sodium hydroxide;
1 mg/mL to 25 mg/mL hydrochloric acid;
10 mg/mL to 100 mg/mL trehalose
0.3 mg/mL to 15 mg/mL sodium chloride.

147. The pharmaceutical formulation according to any one of the preceding clauses, wherein the pharmaceutical formulation comprises
0.77 mg/mL to 7.7 mg/mL PEG-ADM,
2 mg/mL to 10 mg/mL citric acid;
0.8 mg/mL to 4 mg/mL sodium hydroxide;
5 mg/mL to 15 mg/mL hydrochloric acid;
30 mg/mL to 70 mg/mL trehalose
0.5 mg/mL to 5 mg/mL sodium chloride.

148. The pharmaceutical formulation according to any one of the preceding clauses, wherein the pharmaceutical formulation comprises
2.31 mg/mL to 3.85 mg/mL PEG-ADM,
4 mg/mL to 7 mg/mL citric acid;
1.5 mg/mL to 3 mg/mL sodium hydroxide;
7 mg/mL to 9 mg/mL hydrochloric acid;
40 mg/mL to 60 mg/mL trehalose
0.5 mg/mL to 2.5 mg/mL sodium chloride.

149. The pharmaceutical formulation according to any one of the preceding clauses, wherein the citric acid is a salt, pharmaceutical acceptable salt, derivative of citric acid is selected from the group consisting of citric acid anhydrous, sodium citrate and citric acid monohydrate.

150. Pharmaceutical formulation comprising the lyophilizate and a solvent, wherein the lyophilizate is a lyophilizate according to any one of clauses.

151. Pharmaceutical formulation according to clause 150, wherein the pharmaceutical formulation is defined according to any one of clauses.

152. The pharmaceutical formulation according to any one of the preceding clauses, wherein the pharmaceutical formulation comprises PEG-ADM in a concentration according to any one of clauses, wherein the concentrations of components are based on the total volume of the liquid pharmaceutical formulation.

153. The pharmaceutical formulation according to any one of the preceding clauses, wherein the pharmaceutical formulation is a reconstituted lyophilizate.

154. The pharmaceutical formulation according to any one of the preceding clauses, wherein the pharmaceutical formulation comprises 0.077 mg/mL to 3.7 mg/mL PEG-ADM.

155. The pharmaceutical formulation according to any one of the preceding clauses, wherein the pharmaceutical formulation comprises 0.077 mg/mL to 3.6 mg/mL PEG-ADM.

156. The pharmaceutical formulation according to any one of the preceding clauses, wherein the pharmaceutical formulation comprises 0.077 mg/mL to 3.5 mg/mL PEG-ADM.

157. The pharmaceutical formulation according to any one of the preceding clauses, wherein the pharmaceutical formulation comprises 0.077 mg/mL to 3.4 mg/mL PEG-ADM.

158. The pharmaceutical formulation according to any one of the preceding clauses, wherein the pharmaceutical formulation comprises 0.077 mg/mL to 3.3 mg/mL PEG-ADM.

159. The pharmaceutical formulation according to any one of the preceding clauses, wherein the pharmaceutical formulation comprises 0.077 mg/mL to 3.2 mg/mL PEG-ADM.

160. The pharmaceutical formulation according to any one of the preceding clauses, wherein the pharmaceutical formulation comprises 0.077 mg/mL to 3.1 mg/mL PEG-ADM.

161. The pharmaceutical formulation according to any one of the preceding clauses, wherein the pharmaceutical formulation comprises 0.077 mg/mL to 3 mg/mL PEG-ADM.

162. The pharmaceutical formulation according to any one of the preceding clauses, wherein the pharmaceutical formulation comprises 0.077 mg/mL to 2.9 mg/mL PEG-ADM.

163. The pharmaceutical formulation according to any one of the preceding clauses, wherein the pharmaceutical formulation comprises 0.077 mg/mL to 2.8 mg/mL PEG-ADM.

164. The pharmaceutical formulation according to any one of the preceding clauses, wherein the pharmaceutical formulation comprises 0.077 mg/mL to 2.7 mg/mL PEG-ADM.

165. The pharmaceutical formulation according to any one of the preceding clauses, wherein the pharmaceutical formulation comprises 0.077 mg/mL to 2.6 mg/mL PEG-ADM.

166. The pharmaceutical formulation according to any one of the preceding clauses, wherein the pharmaceutical formulation comprises 0.077 mg/mL to 2.5 mg/mL PEG-ADM.

167. The pharmaceutical formulation according to any one of the preceding clauses, wherein the pharmaceutical formulation comprises 0.077 mg/mL to 2.4 mg/mL PEG-ADM.

168. The pharmaceutical formulation according to any one of the preceding clauses, wherein the pharmaceutical formulation comprises 0.077 mg/mL to 2.3 mg/mL PEG-ADM.

169. The pharmaceutical formulation according to any one of the preceding clauses, wherein the pharmaceutical formulation comprises 0.077 mg/mL to 2.2 mg/mL PEG-ADM.

170. The pharmaceutical formulation according to any one of the preceding clauses, wherein the pharmaceutical formulation comprises 0.077 mg/mL to 2.1 mg/mL PEG-ADM.

171. The pharmaceutical formulation according to any one of the preceding clauses, wherein the pharmaceutical formulation comprises 0.077 mg/mL to 2 mg/mL PEG-ADM.

172. The pharmaceutical formulation according to any one of the preceding clauses, wherein the pharmaceutical formulation comprises 0.077 mg/mL to 1.9 mg/mL PEG-ADM.

173. The pharmaceutical formulation according to any one of the preceding clauses, wherein the pharmaceutical formulation comprises 0.01 mg/mL to 1.9 mg/mL PEG-ADM.

174. The pharmaceutical formulation according to any one of the preceding clauses, wherein the pharmaceutical formulation comprises 0.077 mg/mL to 3.7 mg/mL PEG-ADM.

175. The pharmaceutical formulation according to any one of the preceding clauses, wherein the pharmaceutical formulation comprises 0.077 mg/mL to 3.6 mg/mL PEG-ADM.

176. The pharmaceutical formulation according to any one of the preceding clauses, wherein the pharmaceutical formulation comprises 0.077 mg/mL to 3.5 mg/mL PEG-ADM.

177. The pharmaceutical formulation according to any one of the preceding clauses, wherein the pharmaceutical formulation comprises 0.077 mg/mL to 3.4 mg/mL PEG-ADM.

178. The pharmaceutical formulation according to any one of the preceding clauses, wherein the pharmaceutical formulation comprises 0.077 mg/mL to 3.3 mg/mL PEG-ADM.

179. The pharmaceutical formulation according to any one of the preceding clauses, wherein the pharmaceutical formulation comprises 0.077 mg/mL to 3.2 mg/mL PEG-ADM.

180. The pharmaceutical formulation according to any one of the preceding clauses, wherein the pharmaceutical formulation comprises 0.077 mg/mL to 3.1 mg/mL PEG-ADM.

181. The pharmaceutical formulation according to any one of the preceding clauses, wherein the pharmaceutical formulation comprises 0.077 mg/mL to 3 mg/mL PEG-ADM.

182. The pharmaceutical formulation according to any one of the preceding clauses, wherein the pharmaceutical formulation comprises 0.077 mg/mL to 2.9 mg/mL PEG-ADM.

183. The pharmaceutical formulation according to any one of the preceding clauses, wherein the pharmaceutical formulation comprises 0.077 mg/mL to 2.8 mg/mL PEG-ADM.

184. The pharmaceutical formulation according to any one of the preceding clauses, wherein the pharmaceutical formulation comprises 0.077 mg/mL to 2.7 mg/mL PEG-ADM.

185. The pharmaceutical formulation according to any one of the preceding clauses, wherein the pharmaceutical formulation comprises 0.077 mg/mL to 2.6 mg/mL PEG-ADM.

186. The pharmaceutical formulation according to any one of the preceding clauses, wherein the pharmaceutical formulation comprises 0.077 mg/mL to 2.5 mg/mL PEG-ADM.

187. The pharmaceutical formulation according to any one of the preceding clauses, wherein the pharmaceutical formulation comprises 0.077 mg/mL to 2.4 mg/mL PEG-ADM.

188. The pharmaceutical formulation according to any one of the preceding clauses, wherein the pharmaceutical formulation comprises 0.077 mg/mL to 2.3 mg/mL PEG-ADM.

189. The pharmaceutical formulation according to any one of the preceding clauses, wherein the pharmaceutical formulation comprises 0.077 mg/mL to 2.2 mg/mL PEG-ADM.

190. The pharmaceutical formulation according to any one of the preceding clauses, wherein the pharmaceutical formulation comprises 0.077 mg/mL to 2.1 mg/mL PEG-ADM.

191. The pharmaceutical formulation according to any one of the preceding clauses, wherein the pharmaceutical formulation comprises 0.077 mg/mL to 2 mg/mL PEG-ADM.

192. The pharmaceutical formulation according to any one of the preceding clauses, wherein the pharmaceutical formulation comprises 0.077 mg/mL to 1.9 mg/mL PEG-ADM.

193. The pharmaceutical formulation according to any one of the preceding clauses, wherein the pharmaceutical formulation comprises 0.077 mg/mL to 1.9 mg/mL PEG-ADM.

194. The pharmaceutical formulation according to any one of the preceding clauses, wherein the pharmaceutical formulation comprises 0.385 mg/mL to 3.7 mg/mL PEG-ADM.

195. The pharmaceutical formulation according to any one of the preceding clauses, wherein the pharmaceutical formulation comprises 0.385 mg/mL to 3.6 mg/mL PEG-ADM.

196. The pharmaceutical formulation according to any one of the preceding clauses, wherein the pharmaceutical formulation comprises 0.385 mg/mL to 3.5 mg/mL PEG-ADM.

197. The pharmaceutical formulation according to any one of the preceding clauses, wherein the pharmaceutical formulation comprises 0.385 mg/mL to 3.4 mg/mL PEG-ADM.

198. The pharmaceutical formulation according to any one of the preceding clauses, wherein the pharmaceutical formulation comprises 0.385 mg/mL to 3.3 mg/mL PEG-ADM.

199. The pharmaceutical formulation according to any one of the preceding clauses, wherein the pharmaceutical formulation comprises 0.385 mg/mL to 3.2 mg/mL PEG-ADM.

200. The pharmaceutical formulation according to any one of the preceding clauses, wherein the pharmaceutical formulation comprises 0.385 mg/mL to 3.1 mg/mL PEG-ADM.

201. The pharmaceutical formulation according to any one of the preceding clauses, wherein the pharmaceutical formulation comprises 0.385 mg/mL to 3 mg/mL PEG-ADM.

202. The pharmaceutical formulation according to any one of the preceding clauses, wherein the pharmaceutical formulation comprises 0.385 mg/mL to 2.9 mg/mL PEG-ADM.

203. The pharmaceutical formulation according to any one of the preceding clauses, wherein the pharmaceutical formulation comprises 0.385 mg/mL to 2.8 mg/mL PEG-ADM.

204. The pharmaceutical formulation according to any one of the preceding clauses, wherein the pharmaceutical formulation comprises 0.385 mg/mL to 2.7 mg/mL PEG-ADM.

205. The pharmaceutical formulation according to any one of the preceding clauses, wherein the pharmaceutical formulation comprises 0.385 mg/mL to 2.6 mg/mL PEG-ADM.

206. The pharmaceutical formulation according to any one of the preceding clauses, wherein the pharmaceutical formulation comprises 0.385 mg/mL to 2.5 mg/mL PEG-ADM.

207. The pharmaceutical formulation according to any one of the preceding clauses, wherein the pharmaceutical formulation comprises 0.385 mg/mL to 2.4 mg/mL PEG-ADM.

208. The pharmaceutical formulation according to any one of the preceding clauses, wherein the pharmaceutical formulation comprises 0.385 mg/mL to 2.3 mg/mL PEG-ADM.

209. The pharmaceutical formulation according to any one of the preceding clauses, wherein the pharmaceutical formulation comprises 0.385 mg/mL to 2.2 mg/mL PEG-ADM.

210. The pharmaceutical formulation according to any one of the preceding clauses, wherein the pharmaceutical formulation comprises 0.385 mg/mL to 2.1 mg/mL PEG-ADM.

211. The pharmaceutical formulation according to any one of the preceding clauses, wherein the pharmaceutical formulation comprises 0.385 mg/mL to 2 mg/mL PEG-ADM.

212. The pharmaceutical formulation according to any one of the preceding clauses, wherein the pharmaceutical formulation comprises 0.385 mg/mL to 1.9 mg/mL PEG-ADM.

213. The pharmaceutical formulation according to any one of the preceding clauses, wherein the pharmaceutical formulation comprises 0.01 mg/mL to 1.9 mg/mL PEG-ADM.

214. The pharmaceutical formulation according to any one of the preceding clauses, wherein the pharmaceutical formulation comprises 0.385 mg/mL to 3.7 mg/mL PEG-ADM.

215. The pharmaceutical formulation according to any one of the preceding clauses, wherein the pharmaceutical formulation comprises 0.385 mg/mL to 3.6 mg/mL PEG-ADM.

216. The pharmaceutical formulation according to any one of the preceding clauses, wherein the pharmaceutical formulation comprises 0.385 mg/mL to 3.5 mg/mL PEG-ADM.

217. The pharmaceutical formulation according to any one of the preceding clauses, wherein the pharmaceutical formulation comprises 0.385 mg/mL to 3.4 mg/mL PEG-ADM.

218. The pharmaceutical formulation according to any one of the preceding clauses, wherein the pharmaceutical formulation comprises 0.385 mg/mL to 3.3 mg/mL PEG-ADM.

219. The pharmaceutical formulation according to any one of the preceding clauses, wherein the pharmaceutical formulation comprises 0.385 mg/mL to 3.2 mg/mL PEG-ADM.

220. The pharmaceutical formulation according to any one of the preceding clauses, wherein the pharmaceutical formulation comprises 0.385 mg/mL to 3.1 mg/mL PEG-ADM.

221. The pharmaceutical formulation according to any one of the preceding clauses, wherein the pharmaceutical formulation comprises 0.385 mg/mL to 3 mg/mL PEG-ADM.

222. The pharmaceutical formulation according to any one of the preceding clauses, wherein the pharmaceutical formulation comprises 0.385 mg/mL to 2.9 mg/mL PEG-ADM.

223. The pharmaceutical formulation according to any one of the preceding clauses, wherein the pharmaceutical formulation comprises 0.385 mg/mL to 2.8 mg/mL PEG-ADM.

224. The pharmaceutical formulation according to any one of the preceding clauses, wherein the pharmaceutical formulation comprises 0.385 mg/mL to 2.7 mg/mL PEG-ADM.

225. The pharmaceutical formulation according to any one of the preceding clauses, wherein the pharmaceutical formulation comprises 0.385 mg/mL to 2.6 mg/mL PEG-ADM.

226. The pharmaceutical formulation according to any one of the preceding clauses, wherein the pharmaceutical formulation comprises 0.385 mg/mL to 2.5 mg/mL PEG-ADM.

227. The pharmaceutical formulation according to any one of the preceding clauses, wherein the pharmaceutical formulation comprises 0.385 mg/mL to 2.4 mg/mL PEG-ADM.

228. The pharmaceutical formulation according to any one of the preceding clauses, wherein the pharmaceutical formulation comprises 0.385 mg/mL to 2.3 mg/mL PEG-ADM.

229. The pharmaceutical formulation according to any one of the preceding clauses, wherein the pharmaceutical formulation comprises 0.385 mg/mL to 2.2 mg/mL PEG-ADM.

230. The pharmaceutical formulation according to any one of the preceding clauses, wherein the pharmaceutical formulation comprises 0.385 mg/mL to 2.1 mg/mL PEG-ADM.

231. The pharmaceutical formulation according to any one of the preceding clauses, wherein the pharmaceutical formulation comprises 0.385 mg/mL to 2 mg/mL PEG-ADM.

232. The pharmaceutical formulation according to any one of the preceding clauses, wherein the pharmaceutical formulation comprises 0.385 mg/mL to 1.9 mg/mL PEG-ADM.

233. The pharmaceutical formulation according to any one of the preceding clauses, wherein the pharmaceutical formulation comprises 0.385 mg/mL to 1.9 mg/mL PEG-ADM.

234. The pharmaceutical formulation according to any one of the preceding clauses, wherein the pharmaceutical formulation comprises 0.77 mg/mL to 3.7 mg/mL PEG-ADM.

235. The pharmaceutical formulation according to any one of the preceding clauses, wherein the pharmaceutical formulation comprises 0.77 mg/mL to 3.6 mg/mL PEG-ADM.

236. The pharmaceutical formulation according to any one of the preceding clauses, wherein the pharmaceutical formulation comprises 0.77 mg/mL to 3.5 mg/mL PEG-ADM.

237. The pharmaceutical formulation according to any one of the preceding clauses, wherein the pharmaceutical formulation comprises 0.77 mg/mL to 3.4 mg/mL PEG-ADM.238. The pharmaceutical formulation according to any one of the preceding clauses, wherein the pharmaceutical formulation comprises 0.77 mg/mL to 3.3 mg/mL PEG-ADM.

239. The pharmaceutical formulation according to any one of the preceding clauses, wherein the pharmaceutical formulation comprises 0.77 mg/mL to 3.2 mg/mL PEG-ADM.

240. The pharmaceutical formulation according to any one of the preceding clauses, wherein the pharmaceutical formulation comprises 0.77 mg/mL to 3.1 mg/mL PEG-ADM.

241. The pharmaceutical formulation according to any one of the preceding clauses, wherein the pharmaceutical formulation comprises 0.77 mg/mL to 3 mg/mL PEG-ADM.

242. The pharmaceutical formulation according to any one of the preceding clauses, wherein the pharmaceutical formulation comprises 0.77 mg/mL to 2.9 mg/mL PEG-ADM.

243. The pharmaceutical formulation according to any one of the preceding clauses, wherein the pharmaceutical formulation comprises 0.77 mg/mL to 2.8 mg/mL PEG-ADM.

244. The pharmaceutical formulation according to any one of the preceding clauses, wherein the pharmaceutical formulation comprises 0.77 mg/mL to 2.7 mg/mL PEG-ADM.

245. The pharmaceutical formulation according to any one of the preceding clauses, wherein the pharmaceutical formulation comprises 0.77 mg/mL to 2.6 mg/mL PEG-ADM.

246. The pharmaceutical formulation according to any one of the preceding clauses, wherein the pharmaceutical formulation comprises 0.77 mg/mL to 2.5 mg/mL PEG-ADM.

247. The pharmaceutical formulation according to any one of the preceding clauses, wherein the pharmaceutical formulation comprises 0.77 mg/mL to 2.4 mg/mL PEG-ADM.

248. The pharmaceutical formulation according to any one of the preceding clauses, wherein the pharmaceutical formulation comprises 0.77 mg/mL to 2.3 mg/mL PEG-ADM.

249. The pharmaceutical formulation according to any one of the preceding clauses, wherein the pharmaceutical formulation comprises 0.77 mg/mL to 2.2 mg/mL PEG-ADM.

250. The pharmaceutical formulation according to any one of the preceding clauses, wherein the pharmaceutical formulation comprises 0.77 mg/mL to 2.1 mg/mL PEG-ADM.

251. The pharmaceutical formulation according to any one of the preceding clauses, wherein the pharmaceutical formulation comprises 0.77 mg/mL to 2 mg/mL PEG-ADM.

252. The pharmaceutical formulation according to any one of the preceding clauses, wherein the pharmaceutical formulation comprises 0.77 mg/mL to 1.9 mg/mL PEG-ADM.

253. The pharmaceutical formulation according to any one of the preceding clauses, wherein the pharmaceutical formulation comprises 0.01 mg/mL to 1.9 mg/mL PEG-ADM.

254. The pharmaceutical formulation according to any one of the preceding clauses, wherein the pharmaceutical formulation comprises 0.77 mg/mL to 3.7 mg/mL PEG-ADM.

255. The pharmaceutical formulation according to any one of the preceding clauses, wherein the pharmaceutical formulation comprises 0.77 mg/mL to 3.6 mg/mL PEG-ADM.

256. The pharmaceutical formulation according to any one of the preceding clauses, wherein the pharmaceutical formulation comprises 0.77 mg/mL to 3.5 mg/mL PEG-ADM.

257. The pharmaceutical formulation according to any one of the preceding clauses, wherein the pharmaceutical formulation comprises 0.77 mg/mL to 3.4 mg/mL PEG-ADM.

258. The pharmaceutical formulation according to any one of the preceding clauses, wherein the pharmaceutical formulation comprises 0.77 mg/mL to 3.3 mg/mL PEG-ADM.

259. The pharmaceutical formulation according to any one of the preceding clauses, wherein the pharmaceutical formulation comprises 0.77 mg/mL to 3.2 mg/mL PEG-ADM.

260. The pharmaceutical formulation according to any one of the preceding clauses, wherein the pharmaceutical formulation comprises 0.77 mg/mL to 3.1 mg/mL PEG-ADM.

261. The pharmaceutical formulation according to any one of the preceding clauses, wherein the pharmaceutical formulation comprises 0.77 mg/mL to 3 mg/mL PEG-ADM.

262. The pharmaceutical formulation according to any one of the preceding clauses, wherein the pharmaceutical formulation comprises 0.77 mg/mL to 2.9 mg/mL PEG-ADM.

263. The pharmaceutical formulation according to any one of the preceding clauses, wherein the pharmaceutical formulation comprises 0.77 mg/mL to 2.8 mg/mL PEG-ADM.

264. The pharmaceutical formulation according to any one of the preceding clauses, wherein the pharmaceutical formulation comprises 0.77 mg/mL to 2.7 mg/mL PEG-ADM.

265. The pharmaceutical formulation according to any one of the preceding clauses, wherein the pharmaceutical formulation comprises 0.77 mg/mL to 2.6 mg/mL PEG-ADM.

266. The pharmaceutical formulation according to any one of the preceding clauses, wherein the pharmaceutical formulation comprises 0.77 mg/mL to 2.5 mg/mL PEG-ADM.

267. The pharmaceutical formulation according to any one of the preceding clauses, wherein the pharmaceutical formulation comprises 0.77 mg/mL to 2.4 mg/mL PEG-ADM.

268. The pharmaceutical formulation according to any one of the preceding clauses, wherein the pharmaceutical formulation comprises 0.77 mg/mL to 2.3 mg/mL PEG-ADM.

269. The pharmaceutical formulation according to any one of the preceding clauses, wherein the pharmaceutical formulation comprises 0.77 mg/mL to 2.2 mg/mL PEG-ADM.

270. The pharmaceutical formulation according to any one of the preceding clauses, wherein the pharmaceutical formulation comprises 0.77 mg/mL to 2.1 mg/mL PEG-ADM.

271. The pharmaceutical formulation according to any one of the preceding clauses, wherein the pharmaceutical formulation comprises 0.77 mg/mL to 2 mg/mL PEG-ADM.

272. The pharmaceutical formulation according to any one of the preceding clauses, wherein the pharmaceutical formulation comprises 0.77 mg/mL to 1.9 mg/mL PEG-ADM.

273. The pharmaceutical formulation according to any one of the preceding clauses, wherein the pharmaceutical formulation comprises 0.77 mg/mL to 1.9 mg/mL PEG-ADM.

274. The pharmaceutical formulation according to any one of the preceding clauses, wherein the pharmaceutical formulation comprises 1.5 mg/mL to 3.7 mg/mL PEG-ADM.

275. The pharmaceutical formulation according to any one of the preceding clauses, wherein the pharmaceutical formulation comprises 1.5 mg/mL to 3.6 mg/mL PEG-ADM.

276. The pharmaceutical formulation according to any one of the preceding clauses, wherein the pharmaceutical formulation comprises 1.5 mg/mL to 3.5 mg/mL PEG-ADM.

277. The pharmaceutical formulation according to any one of the preceding clauses, wherein the pharmaceutical formulation comprises 1.5 mg/mL to 3.4 mg/mL PEG-ADM.

278. The pharmaceutical formulation according to any one of the preceding clauses, wherein the pharmaceutical formulation comprises 1.5 mg/mL to 3.3 mg/mL PEG-ADM.

279. The pharmaceutical formulation according to any one of the preceding clauses, wherein the pharmaceutical formulation comprises 1.5 mg/mL to 3.2 mg/mL PEG-ADM.

280. The pharmaceutical formulation according to any one of the preceding clauses, wherein the pharmaceutical formulation comprises 1.5 mg/mL to 3.1 mg/mL PEG-ADM.

281. The pharmaceutical formulation according to any one of the preceding clauses, wherein the pharmaceutical formulation comprises 1.5 mg/mL to 3 mg/mL PEG-ADM.

282. The pharmaceutical formulation according to any one of the preceding clauses, wherein the pharmaceutical formulation comprises 1.5 mg/mL to 2.9 mg/mL PEG-ADM.

283. The pharmaceutical formulation according to any one of the preceding clauses, wherein the pharmaceutical formulation comprises 1.5 mg/mL to 2.8 mg/mL PEG-ADM.

284. The pharmaceutical formulation according to any one of the preceding clauses, wherein the pharmaceutical formulation comprises 1.5 mg/mL to 2.7 mg/mL PEG-ADM.

285. The pharmaceutical formulation according to any one of the preceding clauses, wherein the pharmaceutical formulation comprises 1.5 mg/mL to 2.6 mg/mL PEG-ADM.

286. The pharmaceutical formulation according to any one of the preceding clauses, wherein the pharmaceutical formulation comprises 1.5 mg/mL to 2.5 mg/mL PEG-ADM.

287. The pharmaceutical formulation according to any one of the preceding clauses, wherein the pharmaceutical formulation comprises 1.5 mg/mL to 2.4 mg/mL PEG-ADM.

288. The pharmaceutical formulation according to any one of the preceding clauses, wherein the pharmaceutical formulation comprises 1.5 mg/mL to 2.3 mg/mL PEG-ADM.

289. The pharmaceutical formulation according to any one of the preceding clauses, wherein the pharmaceutical formulation comprises 1.5 mg/mL to 2.2 mg/mL PEG-ADM.

290. The pharmaceutical formulation according to any one of the preceding clauses, wherein the pharmaceutical formulation comprises 1.5 mg/mL to 2.1 mg/mL PEG-ADM.

291. The pharmaceutical formulation according to any one of the preceding clauses, wherein the pharmaceutical formulation comprises 1.5 mg/mL to 2 mg/mL PEG-ADM.

292. The pharmaceutical formulation according to any one of the preceding clauses, wherein the pharmaceutical formulation comprises 1.5 mg/mL to 1.9 mg/mL PEG-ADM.

293. The pharmaceutical formulation according to any one of the preceding clauses, wherein the pharmaceutical formulation comprises 0.01 mg/mL to 1.9 mg/mL PEG-ADM.

294. The pharmaceutical formulation according to any one of the preceding clauses, wherein the pharmaceutical formulation comprises 1.5 mg/mL to 3.7 mg/mL PEG-ADM.

295. The pharmaceutical formulation according to any one of the preceding clauses, wherein the pharmaceutical formulation comprises 1.5 mg/mL to 3.6 mg/mL PEG-ADM.

296. The pharmaceutical formulation according to any one of the preceding clauses, wherein the pharmaceutical formulation comprises 1.5 mg/mL to 3.5 mg/mL PEG-ADM.

297. The pharmaceutical formulation according to any one of the preceding clauses, wherein the pharmaceutical formulation comprises 1.5 mg/mL to 3.4 mg/mL PEG-ADM.

298. The pharmaceutical formulation according to any one of the preceding clauses, wherein the pharmaceutical formulation comprises 1.5 mg/mL to 3.3 mg/mL PEG-ADM.

299. The pharmaceutical formulation according to any one of the preceding clauses, wherein the pharmaceutical formulation comprises 1.5 mg/mL to 3.2 mg/mL PEG-ADM.

300. The pharmaceutical formulation according to any one of the preceding clauses, wherein the pharmaceutical formulation comprises 1.5 mg/mL to 3.1 mg/mL PEG-ADM.

301. The pharmaceutical formulation according to any one of the preceding clauses, wherein the pharmaceutical formulation comprises 1.5 mg/mL to 3 mg/mL PEG-ADM.

302. The pharmaceutical formulation according to any one of the preceding clauses, wherein the pharmaceutical formulation comprises 1.5 mg/mL to 2.9 mg/mL PEG-ADM.

303. The pharmaceutical formulation according to any one of the preceding clauses, wherein the pharmaceutical formulation comprises 1.5 mg/mL to 2.8 mg/mL PEG-ADM.

304. The pharmaceutical formulation according to any one of the preceding clauses, wherein the pharmaceutical formulation comprises 1.5 mg/mL to 2.7 mg/mL PEG-ADM.

305. The pharmaceutical formulation according to any one of the preceding clauses, wherein the pharmaceutical formulation comprises 1.5 mg/mL to 2.6 mg/mL PEG-ADM.

306. The pharmaceutical formulation according to any one of the preceding clauses, wherein the pharmaceutical formulation comprises 1.5 mg/mL to 2.5 mg/mL PEG-ADM.

307. The pharmaceutical formulation according to any one of the preceding clauses, wherein the pharmaceutical formulation comprises 1.5 mg/mL to 2.4 mg/mL PEG-ADM.

308. The pharmaceutical formulation according to any one of the preceding clauses, wherein the pharmaceutical formulation comprises 1.5 mg/mL to 2.3 mg/mL PEG-ADM.

309. The pharmaceutical formulation according to any one of the preceding clauses, wherein the pharmaceutical formulation comprises 1.5 mg/mL to 2.2 mg/mL PEG-ADM.

310. The pharmaceutical formulation according to any one of the preceding clauses, wherein the pharmaceutical formulation comprises 1.5 mg/mL to 2.1 mg/mL PEG-ADM.

311. The pharmaceutical formulation according to any one of the preceding clauses, wherein the pharmaceutical formulation comprises 1.5 mg/mL to 2 mg/mL PEG-ADM.

312. The pharmaceutical formulation according to any one of the preceding clauses, wherein the pharmaceutical formulation comprises 1.5 mg/mL to 1.9 mg/mL PEG-ADM.

313. The pharmaceutical formulation according to any one of the preceding clauses, wherein the pharmaceutical formulation comprises 1.5 mg/mL to 1.9 mg/mL PEG-ADM.

314. The pharmaceutical formulation according to any one of the preceding clauses, wherein the formulation comprises 1 mg/mL to 300 mg/mL trehalose.

315. The pharmaceutical formulation according to any one of the preceding clauses, wherein the formulation comprises 0.1 mg/mL to 200 mg/mL trehalose.

316. The pharmaceutical formulation according to any one of the preceding clauses, wherein the formulation comprises 10 mg/mL to 100 mg/mL trehalose.

317. The pharmaceutical formulation according to any one of the preceding clauses, wherein the formulation comprises 30 mg/mL to 70 mg/mL trehalose.

318. The pharmaceutical formulation according to any one of the preceding clauses, wherein the formulation comprises 40 mg/mL to 60 mg/mL trehalose.

319. The pharmaceutical formulation according to any one of the preceding clauses, wherein the formulation comprises 0.3 mg/mL to 30 mg/mL of a pH regulator.

320. The pharmaceutical formulation according to any one of the preceding clauses, wherein the formulation comprises 1 mg/mL to 15 mg/mL of a pH regulator.

321. The pharmaceutical formulation according to any one of the preceding clauses, wherein the formulation comprises 2 mg/mL to 10 mg/mL of a pH regulator.

322. The pharmaceutical formulation according to any one of the preceding clauses, wherein the formulation comprises 4 mg/mL to 7 mg/mL of a pH regulator.

323. The pharmaceutical formulation according to any one of the preceding clauses, wherein the formulation comprises 0.1 mg/mL to 100 mg/mL citric acid.

324. The pharmaceutical formulation according to any one of the preceding clauses, wherein the formulation comprises 0.3 mg/mL to 30 mg/mL citric acid.

325. The pharmaceutical formulation according to any one of the preceding clauses, wherein the formulation comprises 1 mg/mL to 15 mg/mL citric acid.

326. The pharmaceutical formulation according to any one of the preceding clauses, wherein the formulation comprises 2 mg/mL to 10 mg/mL citric acid.

327. The pharmaceutical formulation according to any one of the preceding clauses, wherein the formulation comprises 4 mg/mL to 7 mg/mL citric acid.

328. Pharmaceutical formulation according to any of the preceding clauses 1 to 327 for inhalation.

329. Medicament comprising the pharmaceutical formulation according to any one of clauses 1 to 327 or a medicament comprising the pharmaceutical formulation according to any one of clauses 1 to 327 in combination with an inert nontoxic pharmaceutically suitable excipient, optionally in combination with a further active ingredient.

330. Combined pharmaceutical dose form comprising components (1) and (2), wherein
component (1) comprises a pharmaceutical formulation according to any one of the preceding clauses; and
component (2) comprises a solvent.

331. The combined pharmaceutical dose form according to clause 330, wherein the component (1) is solution, aqueous formulation, dispersion, reconstituted lyophilizate or lyophilizate according to any one of clauses 1 to 327.

332. The combined pharmaceutical dose form according to any one of clauses, wherein the component (1) is a solution, dispersion, soluble powder, lyophilizate, tablet or granulate, which comprises at least one of the components a, c and/or component d., and component (2) comprises component b for solving or dispersing component (1).

333. Combination pack comprising component (1) and (2), wherein
component (1) comprises the pharmaceutical formulation, the medicament, or the combined pharmaceutical dose form according to any one of clauses 1 to 332; and
component (2) comprises a nebulizer, preferably a mesh nebulizer.

334. The pharmaceutical formulation according to any one of clauses 1 to 327, the medicament according to clause 328, the combined pharmaceutical dose form according to any one of clauses 329 to 332, or the combination pack according to clause 333 for use in the treatment and/or prevention of diseases.

335. The pharmaceutical formulation according to any one of clauses 1 to 327, the medicament according to clause 328, the combined pharmaceutical dose form according to any one of clauses 329 to 332, or the combination pack according to clause 333, the compound of formula (I), the compound according to formula (Ia) for use in the treatment and/or prevention of diseases and/or disorders, wherein the disease and/or disorder is selected from
pulmonary disorders, such as pulmonary hypertension; secondary pulmonary hypertension; pulmonary hypertension following pulmonary embolism with and without acute cor pulmonale; primary pulmonary hypertension; chronic obstructive pulmonary disease; asthma; acute pulmonary edema; chronic pulmonary edema; allergic alveolitis; pneumonitis due to inhaled organic dust; pneumonitis due to inhaled particles of fungal, actinomycetic or other origin; acute chemical bronchitis; acute chemical pulmonary edema and/or chronic chemical pulmonary edema (e.g. after inhalation of phosgene, nitrogen oxide); neurogenic pulmonary edema; acute pulmonary manifestations due to radiation; chronic pulmonary manifestations due to radiation; acute and/or chronic interstitial lung disorders (such as but not restricted to drug-induced interstitial lung disorders, e.g. secondary to Bleomycin treatment); acute lung injury (ALI); acute lung injury (ALI) in adult or child including newborn; acute respiratory distress syndrome (ARDS); acute respiratory distress syndrome (ARDS) in adult or child including newborn; ALI/ARDS secondary to pneumonia and sepsis, aspiration pneumonia and ALI/ARDS secondary to aspiration (such as but not restricted to aspiration pneumonia due to regurgitated gastric content); ALI/ARDS secondary to smoke gas inhalation; transfusion-related acute lung injury (TRALI), ALI/ARDS or acute pulmonary insufficiency following surgery; trauma or burns, ventilator induced lung injury (VILI); lung injury following meconium aspiration; pulmonary fibrosis; and mountain sickness;
ALI/ARDS secondary to pneumonia caused by bacterial infection of the lungs, such as, but not restricted to, bacterial pneumonia caused by Pneumococci, Haemophilus Influenzae, Mycoplasma Pneumoniae, Chlamydia species, Enterococci, beta-hemolytic Streptococci, Staphylococci, Gram-negative Enterobacteriaceae, Pseudomonas species, Klebsiella species, Acinetobacter species, Legionella species, and Mycobacteria;
ALI/ARDS secondary to pneumonia caused by viral infections such as, but not restricted to, Influenza viruses (e.g. caused by strains of serotypes H1N1, H5N1, H7N9), Corona viruses (e.g. SARS-CoV, the pathogen of severe acute respiratory syndrome (SARS), MERS-CoV, the pathogen of Middle East respiratory syndrome (MERS), and SARS-CoV-2 the pathogen of COVID-19 pandemic), Respiratory-Syncytial-Virus (RSV), and Cytomegalovirus (CMV);
ALI/ARDS secondary to pneumonia caused by fungal infections such as, but not restricted to, fungal pneumonia caused by Pneumocystis Jirovecii;
ALI/ARDS secondary to pneumonia irrespective of the context of pneumonia origin such as for community acquired pneumonia (CAP) as well as for hospital acquired pneumonia (HAP), in particular for HAP acquired in the context of artificial ventilation (VAP);
ALI/ARDS secondary to pneumonia irrespective of the diverse pathoanatomical appearances of pneumonias such as, but not restricted to, lobar (i.e. affecting an entire lung lobe), lobular (i.e. affecting smaller lung lobules), interstitial (i.e. diffuse affection of the lung tissue);
ALI/ARDS secondary to pneumonia occurring in consequence of bacterial and/or virus infection;
ALI/ARDS secondary to pneumonia occurring in consequence of a bacterial superinfection of a primary lung affection by viruses; and
prevention and/or treatment of lung dysfunction after lung transplantations.

336. The compound of formula (I)

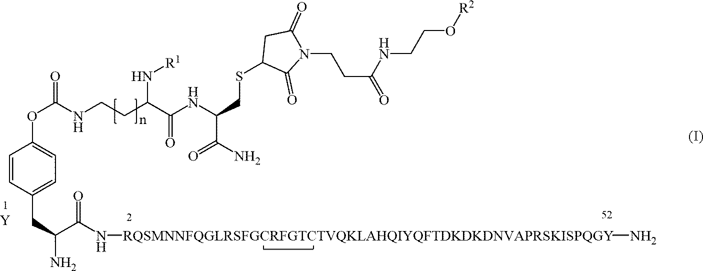

in which
n represents the number 0, 1, 2 or 3, R¹ represents hydrogen, methyl, ethyl, n-propyl or isopropyl;
R² represents linear or branched PEG 20 kDa to 80 kDa endcapped with a methoxy-group; or
a hydrate thereof, solvate thereof, salt thereof, pharmaceutically acceptable salt thereof, or the solvates of salts thereof; as defined in any one of clauses 1 to 124 or the compound according to formula (Ia)

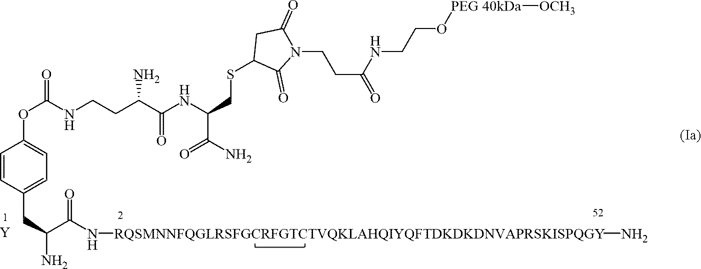

as defined in any one of clauses clauses 1 to 124 for use in the treatment and/or prevention of ALI/ARDS secondary to pneumonia caused by bacterial infection of the lungs, such as, but not restricted to, bacterial pneumonia caused by Pneumococci, Haemophilus Influenzae, Mycoplasma Pneumoniae, Chlamydia species, Enterococci, beta-hemolytic Streptococci, Staphylococci, Gram-negative Enterobacteriaceae, Pseudomonas species, Klebsiella species, Acinetobacter species, Legionella species, and Mycobacteria; ALI/ARDS secondary to pneumonia caused by viral infections such as, but not restricted to, Influenza viruses (e.g. caused by strains of serotypes H1N1, H5N1, H7N9), Corona viruses (e.g. SARS-CoV, the pathogen of severe acute respiratory syndrome (SARS), MERS-CoV, the pathogen of Middle East respiratory syndrome (MERS), and SARS-CoV-2 the pathogen of COVID-19 pandemic), Respiratory-Syncytial-Virus (RSV), and Cytomegalovirus (CMV); ALI/ARDS secondary to pneumonia caused by fungal infections such as, but not restricted to, fungal pneumonia caused by Pneumocystis Jirovecii; ALI/ARDS secondary to pneumonia irrespective of the context of pneumonia origin such as for community acquired pneumonia (CAP) as well as for hospital acquired pneumonia (HAP), in particular for HAP acquired in the context of artificial ventilation (VAP); ALI/ARDS secondary to pneumonia irrespective of the diverse pathoanatomical appearances of pneumonias such as, but not restricted to, lobar (i.e. affecting an entire lung lobe), lobular (i.e. affecting smaller lung lobules), interstitial (i.e. diffuse affection of the lung tissue); ALI/ARDS secondary to pneumonia occurring in consequence of bacterial and/or virus infection; and ALI/ARDS secondary to pneumonia occurring in consequence of a bacterial superinfection of a primary lung affection by viruses.

337. The use of the pharmaceutical formulation according to any one clauses 1 to 327, the medicament according to clause 328, the combined pharmaceutical dose form according to any one of clauses 329 to 332, or the combination pack according to clause 333, the compound of formula (I) or the compound according to formula (Ia) for the treatment and/or prevention of a disease or disorder, preferably selected from the diseases listed in clauses 404 and/or 405.

338. The pharmaceutical formulation according to any one of clauses 1 to 327 for producing a medicament for treatment and/or prevention of a disease or disorder, preferably selected from the diseases listed in clauses 335 and/or 336.

339. Method of treatment and/or prevention of a disorder and/or disease, preferably selected from the diseases listed in clauses 335 and/or 336, comprising administering the pharmaceutical formulation according to any one of clauses 1 to 327, the medicament according to clause 328, the combined pharmaceutical dose form according to any one of clauses 329 to 332, or the combination pack according to clause 333, the compound of formula (I) or the compound according to formula (Ia).

340. Lyophilizate according to any one of the preceding clauses 1 to 9 obtainable by freeze-drying of the liquid pharmaceutical formulation as defined in any one of the preceding clauses 10 to 218.

341. Liquid Pharmaceutical formulation according to any one of clauses 10 to 327 obtainable by mixing the lyophilizate according to any one of clauses 1 to 78 with a solvent.

342. Liquid Pharmaceutical formulation according to clause 341, wherein the solvent is selected from water, sodium solution, citric acid, a buffer and mixtures thereof.

343. A method for the preparation of the pharmaceutical formulation according to any one of clauses 1 to 327, comprising the following steps:
step 1. Providing at least components a, c and e; and
step 2. Mixing the components provided in step 1;
step 3: freeze-drying the pharmaceutical formulation obtained after any one of steps 1 and/or 2
whereby the following pharmaceutical formulation according to any one of clauses 1 to 327 is obtained.

344. The method according to clause 412, wherein the method further comprises step 4 and/ or step 5:
step 4. Adjusting the pH of the pharmaceutical formulation to a pH of 3 to 5; and/or
step 5. Adjusting the osmolarity of the pharmaceutical formulation to an osmotic concentration between 150 - 450 mosmol/l;
wherein step 4 can be carried before, during and/or after step 1, 2 and/or step 5; and/or wherein step 4 can be carried before, during and/or after step 1, 2 and/or step 4.

345. The method according to any one of clauses 343 to 344, wherein the method comprises the following steps
providing an aqueous formulation of PEG-ADM, which comprises citric acid and optionally at least one pH regulator to adjust the pH to 3.5 and 4.5,
optionally followed by concentration of the aqueous formulation of PEG-ADM
freeze-drying of the formulation and
subsequently reconstitution/dilution of the concentrated product by adding a solution of citric acid and/or sodium citrate, optionally at least one pH regulator and an osmolarity regulator and water, and
wherein the pharmaceutical formulation has an osmotic concentration between 150 - 450 mosmol/L; and wherein the pH of the resulting aqueous formulation is between 3.5 and 4.5.

346. The method according to any one of clauses 343 to 345, wherein the method comprises the following steps
providing an aqueous formulation of PEG-ADM, which comprises citric acid and optionally at least one pH regulator to adjust the pH between 3.5 and 4.5,
providing citric acid and/or sodium citrate, optionally at least one pH regulator and an osmolarity regulator and
mixing the solutions provided, and
wherein the pharmaceutical formulation has an osmotic concentration of between 150 - 450 mosmol/l; and wherein the pH of the resulting aqueous formulation is between 3.5 and 4.5.

347. The method according to clause according to any one of clauses 343 to 346, wherein the method further comprises step 6

Step 6 freeze-drying the pharmaceutical formulation obtained after any one of steps 1, 2, 3, 4 and/or 5; wherein step 6 can be carried before, during and/or after step 1, 2, 3, 4 and/or step 5, whereby a lyophilizate according to any one of the preceding clauses is obtained.

348. The method according to any one of clauses 343 to 347, wherein the method further comprises step 7

Step 7 reconstitution of the lyophilizate according to any one of the preceding clauses obtained after any one of steps 1, 2, 3, 4, 5 and/or 6

349. The formulation according any one of clauses 1 to 327 obtainable by the method according to any one of clauses 412 to 417.

350. A method for the preparation of the pharmaceutical formulation according to any one of clauses 1 to 327, comprising the following steps:
step 1. Providing components a, b, c and d; and
step 2. Mixing the components provided in step 1;
whereby the following pharmaceutical formulation according to any one of clauses 1 to 397 is obtained.

351. The method according to clause 350, wherein the method further comprises step 3 and/ or step 4: and/or step 5
step 3. Adjusting the pH of the pharmaceutical formulation to a pH of 3 to 5; and/or
step 4. Adjusting the osmolarity of the pharmaceutical formulation to an osmotic concentration between 150 - 450 mosmol/l;
wherein step 3 can be carried before, during and/or after step 1, 2 and/or step 4; and/or wherein step 4 can be carried before, during and/or after step 1, 2 and/or step 3.

352. The method according to any one of clauses 350 to 351, wherein the method comprises the following steps
providing an aqueous formulation of PEG-ADM, which comprises citric acid and optionally at least one pH regulator to adjust the pH to 3.5 and 4.5,
followed by concentration of the aqueous formulation of PEG-ADM and
subsequently reconstitution/dilution of the concentrated product by adding a solution of citric acid and/or sodium citrate, optionally at least one pH regulator and an osmolarity regulator and water, and
wherein the pharmaceutical formulation has an osmotic concentration between 150 - 450 mosmol/L; and wherein the pH of the resulting aqueous formulation is between 3.5 and 4.5.

353. The method according to any one of clauses 350 to 352, wherein the method comprises the following steps
providing an aqueous formulation of PEG-ADM, which comprises citric acid and optionally at least one pH regulator to adjust the pH to 3.5 and 4.5,
providing citric acid and/or sodium citrate, optionally at least one pH regulator and an osmolarity regulator and
mixing the solutions provided, and
wherein the pharmaceutical formulation has an osmotic concentration of between 150 - 450 mosmol/l; and wherein the pH of the resulting aqueous formulation is between 3.5 and 4.5.

354. The method according to clause according to any one of clauses 350 to 353, wherein the method further comprises step 5

Step 5 at least partially freezing the pharmaceutical formulation obtained after any one of steps 1, 2, 3 and/or 4; wherein step 4 can be carried before, during and/or after step 1, 2, 3 and/or step 4.

355. The formulation according any one of clauses 1 to 327 obtainable by the method according to any one of clauses 350 to 354.

### XI. DESCRIPTION OF THE FIGURES

In FIG. 1a and FIG. 1b, the remaining assay content of PEG-ADM of Examples 1 to 6 are shown (method “RP-HPLC for Assay of PEG-ADM” as described in section C-2).

In FIG. 2, the PEG-ADM remaining assay content of Example 1 and Example 7 are shown (method “RP-HPLC for Assay of PEG-ADM” as described in section C-2). The Examples 1 and 7 were stored at a temperature of 25° C. for 12 months at a relative humidity when stored in the climatic chamber of 60%.

In FIG. 3, the related substances and degradation of Example 1 and Example 7 are shown (method as described in section C-1 “SEC-HPLC for Purity, Monomer Portion”). The Examples 1 and 7 were stored at a temperature of 25° C. for 12 months at a relative humidity when stored in the climatic chamber of 60%.

FIG. 4 depicts the nebulization of isotonic saline solution 1 (cf. method in section C-3).

FIG. 5 depicts the nebulization of Example 8 - run 1 (cf. method in section C-3).

FIG. 6 depicts the nebulization of isotonic saline solution 2 (cf. method in section C-3).

FIG. 7 depicts the nebulization of isotonic saline solution 3 (cf. method in section C-3).

FIG. 8 depicts the nebulization of Example 8 - run 2(cf. method in section C-3).

FIG. 9 depicts the nebulization of isotonic saline solution 4 (cf. method in section C-3).

FIG. 10 depicts the nebulization of Example 8 - run 3 (cf. method in section C-3).

FIG. 11 depicts the nebulization of isotonic saline solution 1 (cf. method in section C-3).

FIG. 12 depicts the nebulization of Example 9 - run 1 (cf. method in section C-3).

FIG. 13 depicts the nebulization of isotonic saline solution 2.

FIG. 14 depicts the nebulization of Example 9 - run 2.

FIG. 15 depicts the nebulization of isotonic saline solution 3.

FIG. 16 depicts the nebulization of Example 9 - run 3.

FIG. 17 depicts the nebulization of reconstituted Example 10 ((a) run 1; (b) run 2; (c) run 3).

FIG. 18 depicts the nebulization of reconstituted Example 12 ((a) run 1; (b) run 2; (c) run 3).

FIG. 19 depicts the nebulization of reconstituted Example 14 ((a) run 1; (b) run 2; (c) run 3).

FIG. 20 depicts the nebulization of reconstituted Example 15 ((a) run 1; (b) run 2; (c) run 3).

### XII. EXAMPLES

The following working examples illustrate the invention. The invention is not restricted to the examples. The percentages in the following tests and examples are, unless stated otherwise, percentages by weight; parts are parts by weight. Solvent ratios, dilution ratios and concentration data for the liquid/liquid solutions are each based on volume. For all examples described below, a 40 kDa PEG-ADM was used (cf. compound according to formula (Ia)). Approx. 7.7 mg of this 40 kDa PEG-ADM equal to 1 mg ADM.

A. Abbreviations

| | |
|---|---|
| ADM | adrenomedullin (human) |
| DSC | differential scanning calorimetry |
| FPF | Fine particle fraction |
| GSD | geometric standard deviation |
| PEG | polyethylene glycol |
| p.a. | pro analysis |
| q.s. | quantum satis |
| VMD | volumetric median diameter |

Nomenclature of amino acids and peptide sequences is according to:

International Union of Pure and Applied Chemistry and International Union of Biochemistry: Nomenclature and Symbolism for Amino Acids and Peptides (Recommendations 1983). In: Pure & Appl. Chem. 56, Vol. 5, 1984, p. 595-624

TABLE 1–1

| | | |
|---|---|---|
| Trivial Name | Symbol | One-letter Symbol |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### B Preparation of Citric Acid- and Sodium Citrate-supplemented PEG-ADM Solutions

### B-1 Preparation of a Pharmaceutical Formulation (lyophilizate; Examples 1 to 7)

Different pharmaceutical formulations (Examples 1 to 7) were prepared. For all examples described below, a 40 kDa PEG-ADM was used (the compound according to formula (Ia)). Approx. 7.7 mg of this 40 kDa PEG-ADM (the compound according to formula (Ia)) equal to 1 mg ADM. The composition of the pharmaceutical formulations and the resulting concentrations of PEG-ADM [ADM] comprised in the final pharmaceutical formulations is listed in table 1-1 below:

Table 1-1 shows the composition of Examples 1 to 7. The concentration of ADM comprised in PEG-ADM is given in squared brackets. When referring to PEG-ADM the compound of formula (Ia) was used. In the PEG-ADM batch used, approx. 7.7 mg PEG-ADM comprised approx.1 mg ADM The citrate buffer pH 4 used for Examples 1 to 6 had the following composition:5.38 mg/mL citric acid anhydrous, 2.242 mg/mL sodium hydroxide, ~8 mg/mL hydrochloric acid 10% in water for injection. The citrate buffer pH 4 used for Example 7 had the following composition: 7.35 mg/mL sodium citrate, 45 mg/mL hydrochloric acid 10% in water for injection.

TABLE 1–2

| Example | formulation |
|---|---|
| 1 | 3.696 mg/mL PEG-ADM [comprising 0.48 mg/mL ADM], 50 mg/mL trehalose dihydrate, 1.78 mg/mL sodium chloride, citrate buffer pH 4 |
| 2 | 3.696 mg/mL PEG-ADM [comprising 0.48 mg/mL ADM], 50 mg/mL trehalose dihydrate, citrate buffer pH 4 |
| 3 | 3.696 mg/mL PEG-ADM [comprising 0.48 mg/mL ADM], 45 mg/mL mannitol, 2.2 mg/mL L-methionine, 1.35 mg/mL sodium edetate, citrate buffer pH 4 |
| 4 | 3.696 mg/mL PEG-ADM [comprising 0.48 mg/mL ADM], 50 mg/mL sucrose, citrate buffer pH4 |
| 5 | 3.696 mg/mL PEG-ADM [comprising 0.48 mg/mL ADM], 50 mg/mL sucrose, 1.5 mg/mL sodium chloride, citrate buffer pH 4 |
| 6 | 3.696 mg/mL PEG-ADM [comprising 0.48 mg/mL ADM], citrate buffer pH 4 |
| 7 | 2.5 mg/mL PEG-ADM [comprising 0.32 mg/mL ADM], 45 mg/mL Mannitol, 1.49 mg/mL L-Methionine, 1.00 mg/mL Sodium calcium edetate, citrate buffer pH 4 |

The density (g/mL), osmolarity (mOsmol/l) and the residual moisture (%) of Examples 1 to 6 is listed in table 2 below. The density was determined using a Anton Paar DMA 48 unit. The osmolar concentration was determined using a cryo-osmometer capable of measuring the freezing temperature reduction, either a Gonotec 030-D3P or a Fiske Associates 210. The residual moisture content was determined with a Karl Fischer oven method using a Metrohm 851 Titrando with Tiamo software and is provided as % water in the dry lyophilizate.

Table 1-2: density (g/mL), osmolarity (mOsmol/l) and the residual moisture (%) of Examples 1 to 6. The density was determined using a Anton Paar DMA 48 unit. The sample was filled into the air-dried U-tubing without any bubbles. The measurement was conducted by the vibration of the U-tubing. The osmolar concentration was determined using a cryo-osmometer capable of measuring the freezing temperature reduction, either a Gonotec 030-D3P or a Fiske Associates 210. Calibration was done with a 300 mOsmol/kg and 850 mOsmol/kg standard. The residual moisture content was determined with a Karl Fischer oven method using a Metrohm 851 Titrando with Tiamo software and is provided as % water in the dry lyophilizate.

| Example | Density [g/mL] | Osmolarity (mOsmol/l) | Residual moisture (%) |
|---|---|---|---|
| 1 | 1.0 | 300 | 0.5 |
| 2 | 1.0209 | 245 | 0.705 |
| 3 | 1.0203 | 355 | 0.680 |
| 4 | 1.0224 | 249 | 0.987 |
| 5 | 1.0235 | 280 | 1.273 |
| 6 | 1.0033 | 93 | 1.441 |
| 7 | 1.06 | approx. 300 | 1.0 |

Examples 1 and 2 are pharmaceutical formulation according to the invention comprising PEG-ADM and trehalose.

Examples 3 to 5 are PEG-ADM formulations comprising a stabilizator different to the trehalose, i.e. mannitol, sucrose.

Example 6 is a formulation comprising the PEG-ADM only.

Example 7 is a formulation comprising PEG-ADM, mannitol, an antioxidant, and a chelating agent.

The formulations were prepared by dissolving the excipients in purified water (formulation buffer), adjusting to pH 4 and mixing this formulation buffer with a stock solution containing approx. 7.7 mg/mL of PEG-ADM. The solutions were sterile-filtered and filled into type 1 glass vials and semi-stoppered with 20 mm lyophilization stoppers. The products according to example 2 - 6 were freeze dried in a Lyostar 3 freeze dryer with three temperature-controlled shelves. The products according to Example 1 and Example 7 were freeze dried in a HOF pilot freeze dryer with 1.2 m² shelf area. The freeze-drying process applied was different depending on the formulation characteristics.

For the formulations according to Example 1, the freezing step was started by cooling the shelf temperature to 5° C. in order to equilibrate the product temperature for 15 minutes. Subsequently the shelf temperature was decreased at 1° C./min to -45° C., and held for 2.5 hours to achieve complete solidification of the solution. The primary drying step was performed at a shelf temperature of -18° C. and a chamber pressure of 90 µbar over 42 hours. The secondary drying step was performed at a shelf temperature of 30° C. and a chamber pressure of 50 µbar for 4 hours.

For the formulations according to Example 2, 4 and 6, the freezing step was started by cooling the shelf temperature to 5° C. in order to equilibrate the product temperature for 15 minutes. Subsequently the shelf temperature was decreased at 1° C./min to -45° C., and held for 2.5 hours to achieve complete solidification of the solution. The primary drying step was performed at a shelf temperature of -23° C. and a chamber pressure of 50 µbar over 52 hours. The secondary drying step was performed at a shelf temperature of 30° C. and a chamber pressure of 50 µbar for 4 hours.

The formulation according to Example 5 was processed identically as those described above for Example 2, 4 and 6, except that the primary drying shelf temperature was -28° C. and the chamber pressure was 65 µbar.

For the formulation according to Example 3, which contains the crystallizable solute Mannitol, the freezing process was performed by cooling the solution to -45° C., but afterwards an annealing hold step at -20° C. for 4 hours was included to ensure quantitative crystallization of Mannitol and prevent vial breakage or modification transformations during storage. The primary drying step was conducted at a shelf temperature of 5° C. and at a chamber pressure of 90 µbar for 20 hours; the secondary drying step was conducted at a shelf temperature of 30° C. and a chamber pressure of 90 µbar for 4 hours.

For the formulation according to Example 7, which contains the crystallizable solute Mannitol, the freezing process was performed by cooling the solution to -45° C., but afterwards an annealing hold step at -20° C. for 2 hours was included to ensure quantitative crystallization of Mannitol and prevent vial breakage or modification transformations during storage. The primary drying step was conducted at a shelf temperature of 10° C. and at a chamber pressure of 100 µbar for 10 hours; the secondary drying step was conducted at a shelf temperature of 40° C. and a chamber pressure of 100 µbar for 8 hours.

### B-2 Preparation of a Pharmaceutical Formulation (lyophilizate; Examples 10 to 15)

Further pharmaceutical formulations (Examples 10 to 15) were prepared. For all examples described below, a 40 kDa PEG-ADM was used (a compound according to formula (Ia)). Approx. 7.7 mg of this 40 kDa PEG (a compound according to formula (Ia)) ADM equal to 1 mg ADM. The composition of the pharmaceutical formulations and the resulting concentrations of PEG-ADM [ADM] comprised in the final pharmaceutical formulations is listed in table 2-1 below:

Table 2-1 shows the composition of Examples 10 to 15. The concentration of ADM comprised in PEG-ADM is given in squared brackets. When referring in PEG-ADM the compound of formula (Ia) was used. In the PEG-ADM batch used, approx. 7.7 mg PEG-ADM comprised approx.1 mg ADM

TABLE 2–1

| COMPOSITION | EXAMPLES | | | | | |
|---|---|---|---|---|---|---|
| | 10 | 11 | 12 | 13 | 14 | 15 |
| Drug substance | mg/mL | mg/mL | mg/mL | mg/mL | mg/mL | mg/mL |
| PEG-ADM [concentration ADM] | 0.60 [0.0779] | 10.50 [1.3636] | 3.696 [0.48] | 3.696 [0.48] | 3.696 [0.48] | 3.696 [0.48] |
| Excipients | | | | | | |
| Citric acid anhydrous | 5.38 | 5.38 | 5.38 | 5.38 | 5.38 | 5.38 |
| Sodium hydroxide | 2.242 | 2.242 | 2.242 | 2.242 | 2.242 | 2.242 |
| Sodium chloride | 1.78 | 1.78 | 1.78 | 1.78 | 1.78 | 1.78 |
| Trehalose dihydrate | 50 | 50 | 19.65 | 30.5 | 74 | 106 |
| Hydrochloric acid 10%: qs | q.s. | q.s. | q.s. | q.s. | q.s. | q.s. |
| purified water (solvent) | q.s. | q.s. | q.s. | q.s. | q.s. | q.s. |

The formulations according to Example 10 and 12 - 15 were prepared by dissolving the excipients in purified water, adjusting to pH 4 and mixing this formulation buffer with a stock solution containing approx. 7.7 mg/mL of PEG-ADM. For Example 11, a higher-concentrated stock solution with 2.847 mg/mL PEG-ADM (compound (Ia)) was used. The solutions were sterile-filtered and filled into type 1 glass vials and semi-stoppered with 20 mm lyophilization stoppers. The products according to Example 10 - 15 were freeze dried in a Lyostar 3 freeze dryer with three temperature-controlled shelves.

The freezing step was started by cooling the shelf temperature to 5° C. in order to equilibrate the product temperature for 20 minutes. Subsequently the shelf temperature was decreased at 0.7° C./min to -45° C. and held for 3 hours to achieve complete solidification of the solution. The primary drying step was performed at a shelf temperature of -25° C. and a chamber pressure of 60 µbar over 55 hours. The secondary drying step was performed at a shelf temperature of 35° C. and a chamber pressure of 60 µbar for 5 hours.

Examples 10 to 15 were reconstituted in 2.2 mL purified water. The density, osmlarity and residual moisture was measured as described before. The density (g/mL), osmolarity (mOsmol/l) and the residual moisture (%) of Examples 10 to 15 is listed in table 2-2 below. The density was determined using a Anton Paar DMA 48 unit. The osmolar concentration was determined using a cryo-osmometer capable of measuring the freezing temperature reduction, either a Gonotec 030-D3P or a Fiske Associates 210. The residual moisture content was determined with a Karl Fischer oven method using a Metrohm 851 Titrando with Tiamo software and is provided as % water in the dry lyophilizate.

TABLE 2-2

density (g/mL), osmolarity (mOsmol/l) and the residual moisture (%) of Examples 10 to 15

| Example | Density [g/mL] | Osmolarity (mOsmol/l) | Residual moisture (%) | Viscosity (mPa*s) |
|---|---|---|---|---|
| 10 | 1.02154 | 294 | 2.091 | 1.0938 |
| 11 | 1.02353 | 310 | 0.141 | 1.6775 |
| 12 | 1.01177 | 206 | 1.482 | 1.1978 |
| 13 | 1.01553 | 238 | 0.919 | 1.2229 |
| 14 | 1.03032 | 365 | 1.170 | 1.3145 |
| 15 | 1.04071 | 477 | 1.081 | 1.3746 |

In summary, the lyophilizates according to Examples 10 to 15 comprising PEG-ADM and trehalose could also be reconstituted.

### C Analysis: Methods

### C-1 Method SEC-HPLC for Purity, Monomer Portion

SEC-HPLC, size exclusion chromatography (SEC-HPLC) with UV detection at 280 nm, analysis via 100 % method comparing peak areas. The separation and quantitation of PEG-ADM (the monomer portion) as well as the dimer and the HMW aggregates (high molecular weight aggregates) are conducted by SEC-HPLC on an SEC-column using the 100 % area method. (Ph. Eur., 2.2.29 (2015), USP <621> (2011)).

Mobile phase is prepared from NaH₂PO₄ monohydrate, NaCl p.a., Water for chromatography, Ethanol HPLC grade, and 25 mM Citrate buffer pH 4.0.

As stationary phase, e.g. Wyatt SEC Protein Column WTC-030S5 with 300 mm length and 7.8 mm inner diameter can be used. An isocratic elution with a flow of 0.5 mL/min is applied at a temperature of 22° C. and a run time of 30 min, the injection volume is 50 µL.

### C-2 Method: RP-HPLC for Assay of PEG-ADM

The separation, quantitation and identification of PEG-ADM as well as the related substances and degradation products are conducted by RP-HPLC on a reversed phase column and UV detection at 280 nm using an external standard method or by 100% area method with UV detection at 210 nm, resp. (Ph. Eur., 2.2.29 (2015), USP <621> (2011)).

Mobile phase is prepared from trifluoracetic acid > 99.0 %, acetonitrile for chromatography, water for chromatography, and 25 mM citrate buffer. A gradient between 0.1 % TFA in water for chromatography and 0.1 % TFA in acetonitrile for chromatography is applied. As stationary phase, e.g. YMC-Triart Bio C4 with 150 mm length and 3.0 mm inner diameter can be used. The column temperature was 40° C. and the run time was 30 minutes, the injection volume was 50 µL.

### C-3 Method: Determination of Nebulization Properties

Nebulization experiments were performed with the vibrating-mesh nebulizer Aerogen® Solo (Aerogen, Ireland) using isotonic saline and aqueous formulations of PEG-ADM. Samples were filtered prior to use. The device was used either with the Aerogen® ProX Controller or the Aerogen® USB controller. The generated APSD (VMD, GSD and FPF) for each nebulizer unit was characterized by means of laser diffraction (HELOS BR, Sympatec, Germany). The filled nebulizer was clamped into a stand and the exit of the nebulizer was positioned ~50 mm from the Fourier lens face and ~30-60 mm from the laser beam axis. The solutions were then nebulized through the laser beam. The aerosol behind the laser beam was extracted by suction to prevent a re-entry of the aerosol droplets into the laser sensing zone. For calculating the VMD and the FPF the Mie Method was used. The output rate was determined gravimetrically in combination with measuring the duration of the nebulization. The GSD was calculated according the following equation

$$G S D = \sqrt{} \frac{{X}_{84}}{{X}_{16}}$$

All measurements with laser diffraction were carried out at least in triplicate and values are presented as the mean averaged from measurements every 30 s for 2 s.

### C-4 Determination of Density (g/mL), Osmolarity (mOsmol/l) and the Residual Moisture (%)

The density was determined using a Anton Paar DMA 48 unit. The sample was filled into the air-dried U-tubing without any bubbles. The measurement was conducted by the vibration of the U-tubing. The osmolar concentration was determined using a cryo-osmometer capable of measuring the freezing temperature reduction, either a Gonotec 030-D3P or a Fiske Associates 210. Calibration was done with a 300 mOsmol/kg and 850 mOsmol/kg standard. The residual moisture content was determined with a Karl Fischer oven method using a Metrohm 851 Titrando with Tiamo software and is provided as % water in the dry lyophilizate.

### D Results

### D-1 Stability Results

### Stability of Examples 2 to 6:

The stability over 22 months of Examples 1 to 6 (lyophilizates) was analyzed. The analysis was performed according to the methods described in section C-1 and C-2. The Examples 1 to 6 were stored at a temperature of 40° C. for 22 months and a relative humidity of 75%. The results are depicted in FIGS. 1a and 1b.

In FIG. 1a and FIG. 1b, the remaining assay content of PEG-ADM of Examples 1 to 6 are shown (method “RP-HPLC for Assay of PEG-ADM” as described in section C-2). From FIG. 1a and FIG. 1b, over a time period of 22 months at a temperature of 40° C. and a relative humidity of 75%:

Examples 1 and 2 show a PEG-ADM content of approx. 80%. Thus, less than 20% of related substances and degradation products were built over the 22-months-period at the stress condition of 40° C. and 75% relative humidity.

Example 3 shows a PEG-ADM content of approx. 70 %. Thus, less than 30% of related substances and degradation products were built over the 22-months-period at the stress condition of 40° C. and 75% relative humidity.

Example 4 shows a PEG-ADM content of approx. 30 %. Thus, less than 70% of related substances and degradation products were built over the 22-months-period at the stress condition of 40° C. and 75% relative humidity.

Example 5 shows a PEG-ADM content of approx. 45 %. Thus, less than 20% of related substances and degradation products were built over the 22-months-period at the stress condition of 40° C. and 75% relative humidity.

Example 6 shows a PEG-ADM content of approx. 75 %. Thus, less than 25 % of related substances and degradation products were built over the 22-months-period at the stress condition of 40° C. and 75% relative humidity.

In summary it can be seen that the trehalose-based formulation according to Example 2 shows excellent stability during storage at 40° C. and 75% relative humidity, which is confirmed for both size exclusion chromatography and RP HPLC. While the sucrose-based formulations indicate relatively good stability in the SEC method, they show the worst performance in the RP HPLC method. This is due to the formation of a sucrose-related degradation product which is formed at the low pH present in the formulation, therefore sucrose is not suitable as a stabilizer. The formulations containing Mannitol as well as the PEG-ADM without stabilizers show clearly worse stability than the trehalose-based formulations.

### Stability of Example 1 and 7:

The stability of Example 1 and 7 were compared. The stability over 22 months of Examples 1 and 7 (lyophilizates) were analyzed. The analysis was performed according to the methods described in section C-1 and C-2. The Examples 1 and 7 were stored at a temperature of 25° C. for 12 months at a relative humidity when stored in the climatic chamber of 60%.

The results are depicted in FIGS. 2 and 3.

In FIG. 2, the PEG-ADM remaining assay content of Example 1 and Example 7 are shown (method “RP-HPLC for Assay of PEG-ADM” as described in section C-2). From the FIG. 2 it can be seen that over a time period of 12 months at a temperature of 25° C. and a relative humidity of 60%, Example 1 consistently shows a normalized PEG-ADM content over approx. 96%. Thus, only a small amount of related substances and degradation products were built over the 12-months-period. This indicates that the pharmaceutical formulations show an excellent stability. In contrast, Example 7 shows a normalized PEG-ADM content of less than 90% after 3 months and of approx. 91% after 6 months storage, which shows that the stability of Example 7 is clearly worse than for Example 1.

In FIG. 3, the related substances and degradation of Example 1 and Example 7 are shown (method as described in section C-1 “SEC-HPLC for Purity, Monomer Portion”). From the FIG. 3 it can be seen that over a time period of 12 months at a temperature of and a relative humidity of 60%, the amount of the monomer portion of PEG-ADM in Example 1 consistently remains over 99% or in other words the respective samples show a loss of content of PEG-ADM of only 1%. Thus, only a very small amount of HMW aggregates (high molecular weight aggregates) as well as low molecular weight fragments were formed over the 12-months-period. This indicates that the pharmaceutical formulations show an excellent stability. In contrast, Example 7 shows a normalized PEG-ADM content of approx. 96% after 6 months storage, which shows that the stability of Example 7 is clearly worse than for Example 1.

These results indicate that the formulation according to Example 1 shows an excellent stability for storage at 25° C. and 60% relative humidity. Example 7 is less stable.

### D-2 Nebulization Results

The following Examples listed in table 3 were investigated:

TABLE 3

Composition of Examples used in the nebulization experiments

| | |
|---|---|
| Example | formulation |
| 8 | 3.696 PEG-ADM (mg/ml) [comprising 0.48 mg/mL ADM], 50 mg/mL trehalose dihydrate, 1.78 mg/mL sodium chloride, citrate buffer pH 4 |
| 9 | 2.464 mg/mL PEG-ADM [comprising 0.32 mg/mL ADM], 33.3 mg/mL trehalose dihydrate, 1.19 mg/mL sodium chloride, citrate buffer pH 4 |
| Placebo 1 | 20 mg/mL trehalose dihydrate, 2.24 mg/mL L-methionine, 04.5 mg/mL sodium chloride, citrate buffer pH 4 |
| Placebo 2 | 20 mg/mL trehalose dihydrate, 4.5 mg/mL sodium chloride, citrate buffer pH 4 |
| Placebo 3 | 50 mg/mL trehalose dihydrate, 2.24 mg/mL L-methionine, 1.23 mg/mL sodium chloride, citrate buffer pH 4 |
| Placebo 4 | 50 mg/mL trehalose dihydrate, 1.78 mg/mL sodium chloride, citrate buffer pH 4 |

For Examples 8 and 9, the lyophilizate of Example 1 was reconstituted thereby giving the concentrations listed in table 3 above. The reconstitution medium was water. Example 8 had the same concentration as Example 1 before lyophilization. Example 9 had approx. ⅔ of the concentration as Example 1 before lyophilization.

In addition, placebo solutions (cf. Placebo 1 to 4), which did not comprise PEG-ADM, were analyzed.

These formulations were nebulized and measured.

For each nebulized sample, the solution was transferred into the reservoir of the Aerogen® Solo nebulizer, and the nebulizer was positioned above the laser diffractometer. Diffraction spectra were recorded at a 30 second interval over the duration of the nebulization. The collected spectra for each sample are displayed in the respective figure to illustrate the development of the volumetric mean diameter over the nebulization duration. The time required to nebulize the complete amount of solution was recorded for each nebulization, and the throughput of solution per minute was calculated. Samples of normal saline solution were nebulized prior to the first samples to determine the inherent droplet size distribution generated by the nebulizer, as well as between samples to assess changes in the performance of the nebulizers.

Three experiments were conducted:
1. Nebulization of the placebo formulations
2. Nebulization of Example 8
3. Nebulization of Example 9

### D1 Nebulization of the Placebo Formulations

The placebo formulations were nebulized multiple times using the same nebulizer head. The nebulization experiment with the placebo formulations was performed to study the influence of trehalose in the pharmaceutical formulation on the nebulization performance of the nebulizers. This experiment served to investigate if trehalose or other excipient used in the formulations provided in the Examples 1 to 7 had any negative impact on the nebulization properties of the identical nebulizer (Aerogen® Solo). The nebulization of the isotonic saline solution (0.9% NaCl solution) was performed prior to nebulizing the placebo formulations in order to determine the base performance of the nebulizer prior to exposure to the placebo formulations.

The placebo formulations 1 to 4 as well as the isotonic saline solution were nebulized and analyzed as indicated above. The results are given in table 4 below.

GSD means geometric standard deviation. “output rate” in gram per minute (g/min) indicates how many grams of solution are nebulized per minute (throughput). “FPF” in percent (%) means fine particle fraction, which indicates the percentage of particles/droplets below 5 µm. Nebulization properties, expressed as volume median diameter (VDM in micrometer (µm)), of the isotonic saline solution and Examples Placebo 1 to 4.

TABLE 4

| Example | VMD [µm] | GSD | Output rate [g/min] | FPF [%] |
|---|---|---|---|---|
| isotonic saline solution | 5.5 | 2.0 | 0.43 | 52.4 |
| Placebo 1 | 5.5 | 1.9 | 0.46 | 51.2 |
| Placebo 2 | 5.5 | 2.0 | 0.45 | 53.4 |
| Placebo 3 | 5.7 | 2.0 | 0.38 | 51.9 |
| Placebo 4 | 5.4 | 2.0 | 0.43 | 54.2 |

The results clearly show that nebulization of the different trehalose-containing placebo solutions does not result in any changes in the performance of the nebulizer. The droplet size as indicated by the VMD remains identical within the measurement reproducibility, which is also reflected in the fine particle fraction. The output rate of solution per minute is also comparable for all placebo formulations tested.

### D2 Nebulization of Example 8

Example 8 was nebulized multiple times using the same nebulizer head. The nebulization experiment with the Example 8 was performed to study if nebulization of the formulation according to Example 8 results in any changes in droplet size or throughput rate during the nebulization, as well as modifications on the performance of the nebulizer for multiple samples nebulized in a row with the identical nebulizer. A new Aerogen® Solo nebulizer was used for the experiment and initially characterized by nebulizing normal saline solution. Afterwards, samples of solutions according to Example 8 were nebulized in total three times, with nebulization of normal saline samples in between to assess changes in the nebulizer caused by the formulation.

Example 8 as well as the isotonic saline solutions were nebulized and analyzed in the order shown in table 5 below. The results are shown in table 5.

GSD means geometric standard deviation. “output rate” in gram per minute (g/min) indicates how many grams of solution are nebulized per minute (throughput). “FPF” in percent (%) means fine particle fraction, which indicates the percentage of particles/droplets below 5 µm. Nebulization properties, expressed as volume median diameter (VDM in micrometer (µm)), of Example 8 and isotonic saline solution.

TABLE 5

| Sample Run Order | VMD [µm] | GSD | Output rate [g/min] | FPF [%] |
|---|---|---|---|---|
| isotonic saline run 1 | 4.01 | 1.9 | 0.27 | 71.4 |
| Example 8 -run 1 | 7.85 | 2.3 | 0.12 | 47.2 |
| isotonic saline run 2 | 5.12 | 2.1 | 0.38 | 57.2 |
| isotonic saline run 3 | 4.84 | 2.0 | 0.34 | 59.8 |
| Example 8 -run 2 | 7.37 | 2.1 | 0.12 | 46.4 |
| isotonic saline run 4 | 5.40 | 2.2 | 0.41 | 55.4 |
| Example 8 -run 3 | 6.56 | 1.9 | 0.13 | 49.6 |

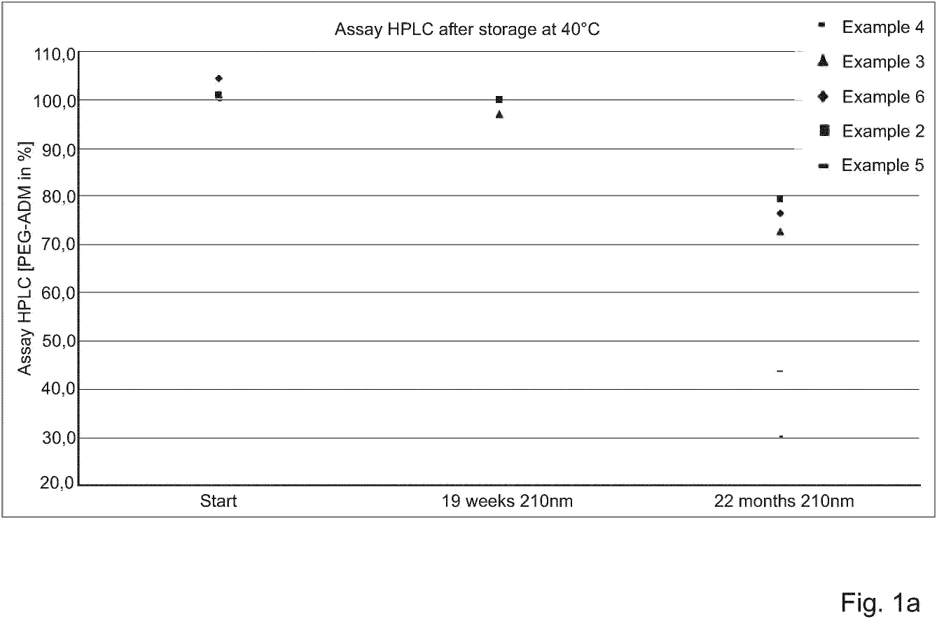

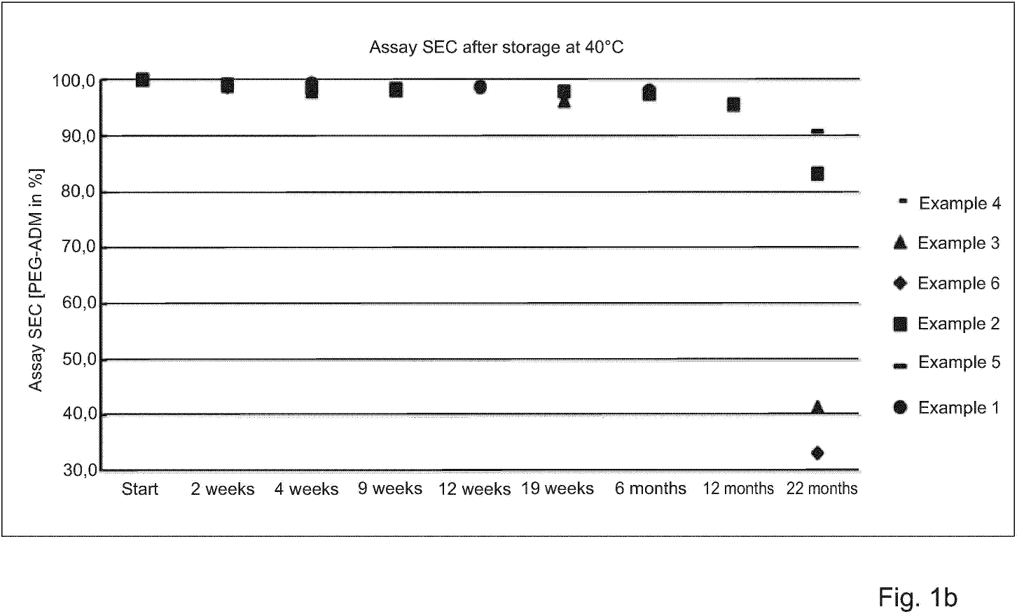

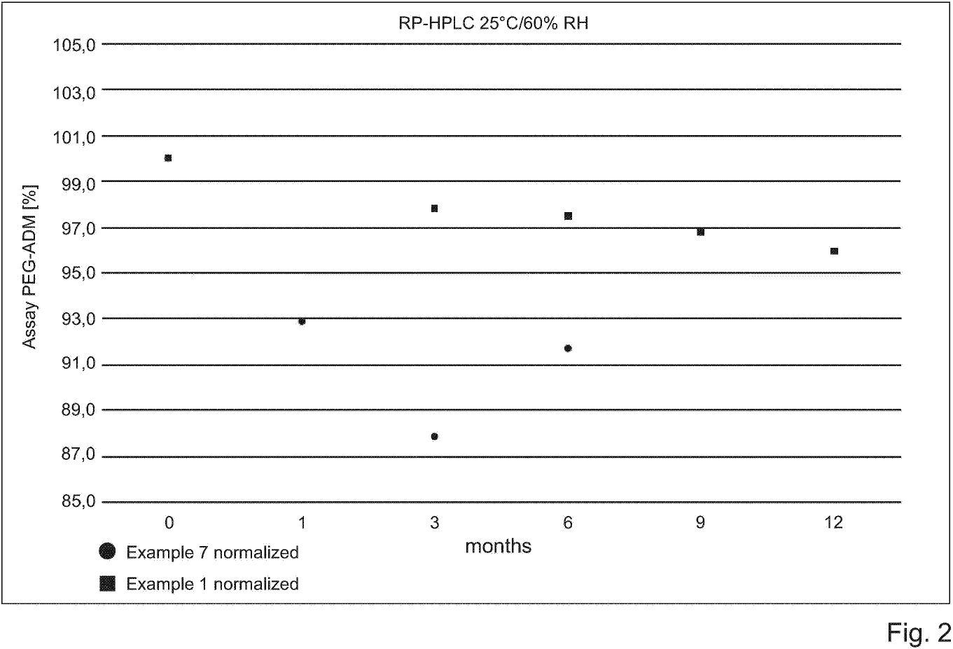

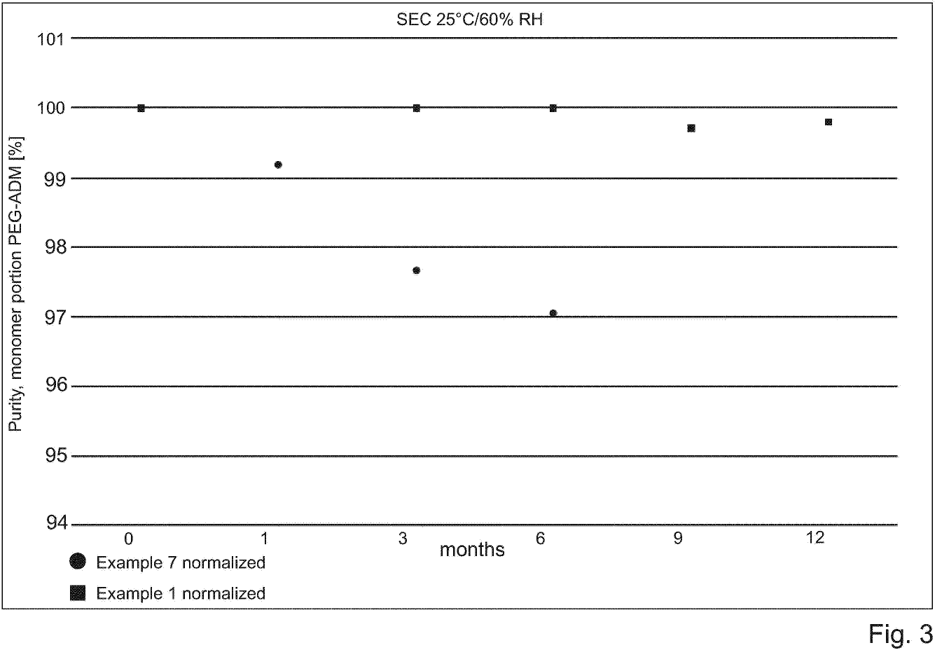

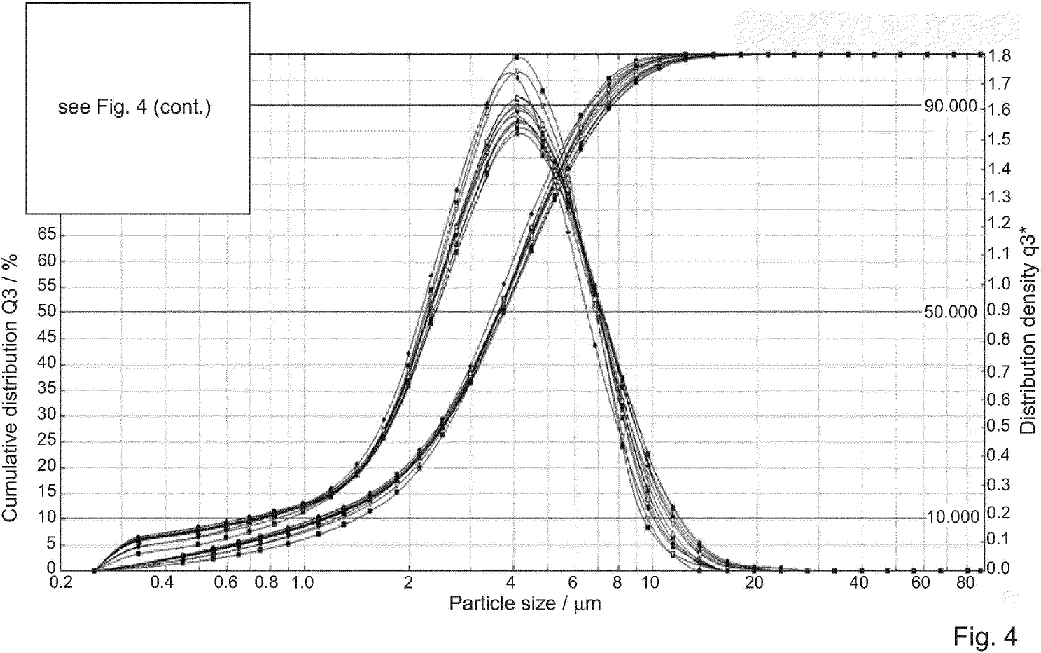

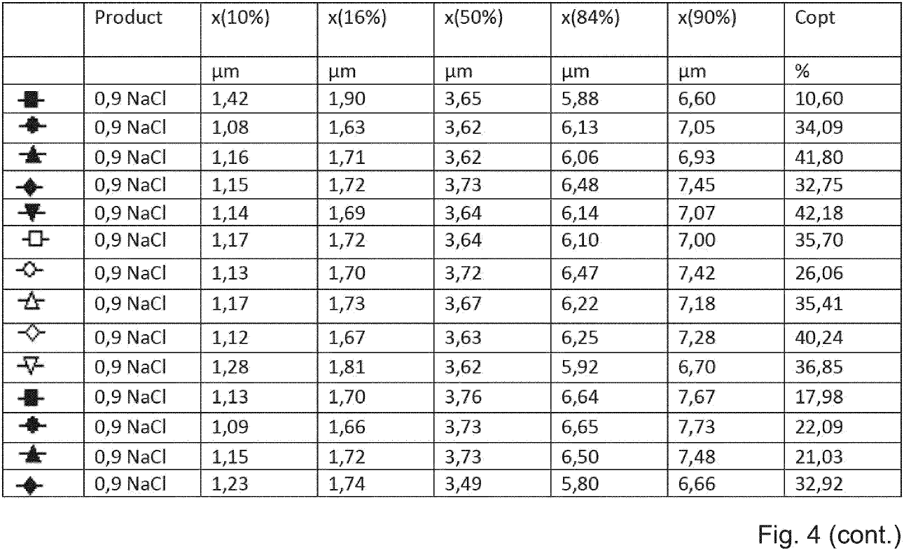

The results are depicted in FIGS. 4 to 10.

FIG. 4 depicts the nebulization of isotonic saline solution 1.

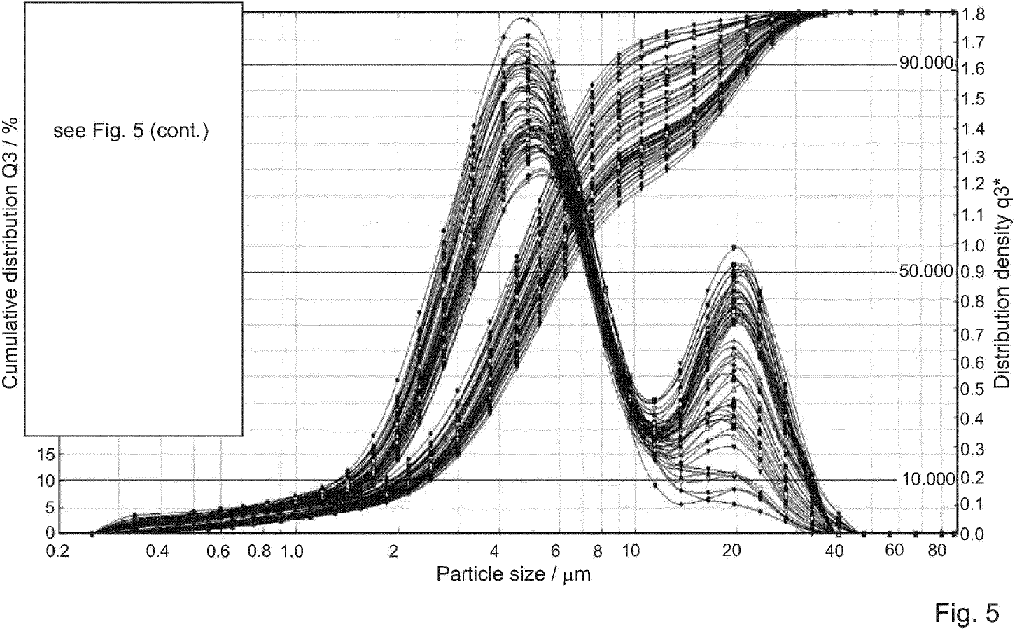

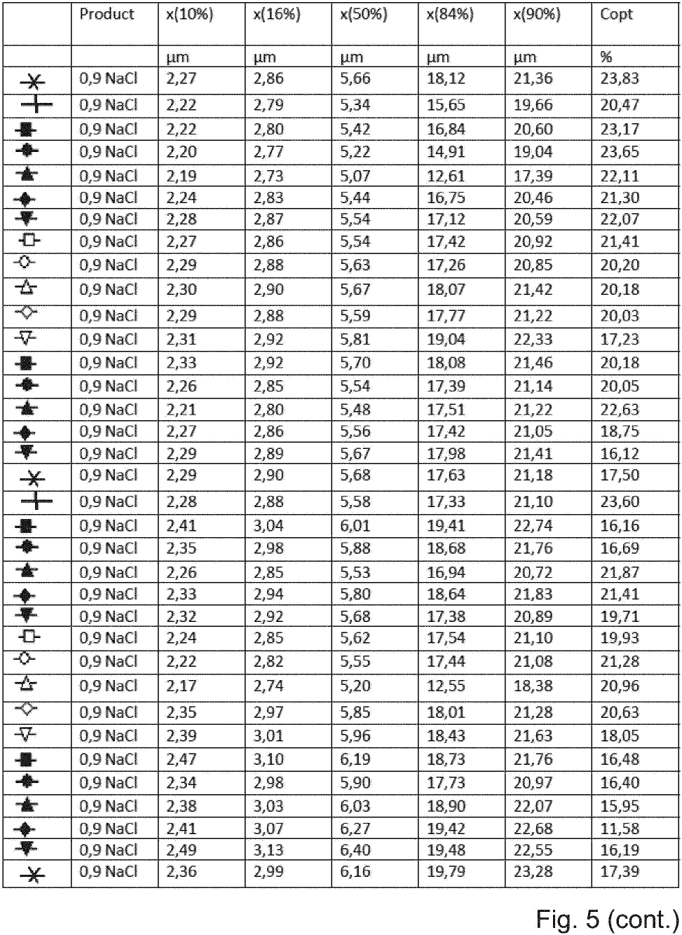

FIG. 5 depicts the nebulization of Example 8 - run 1.

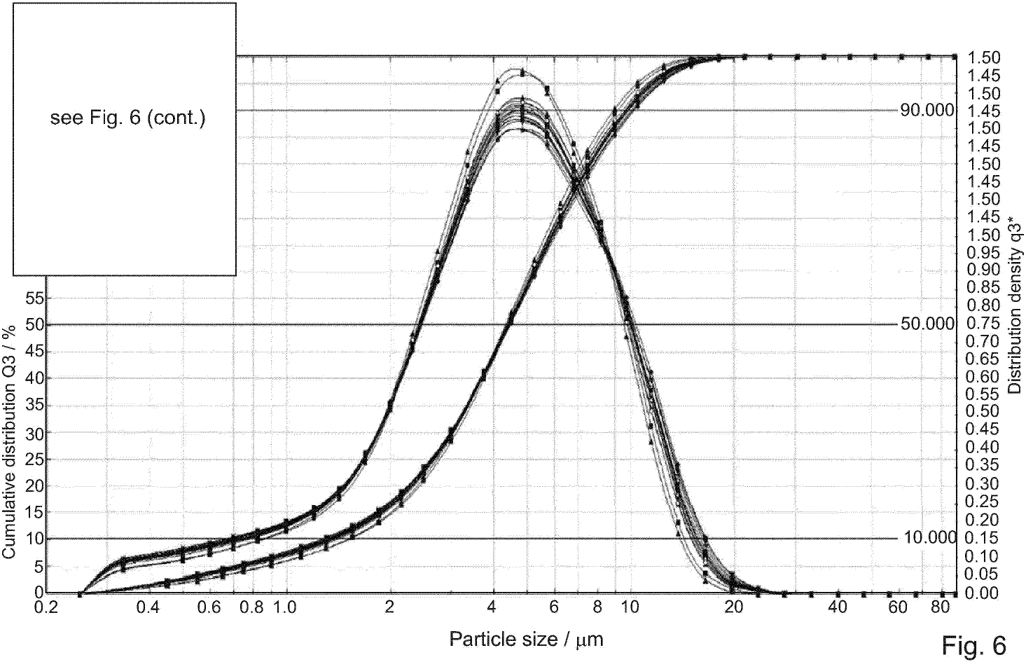

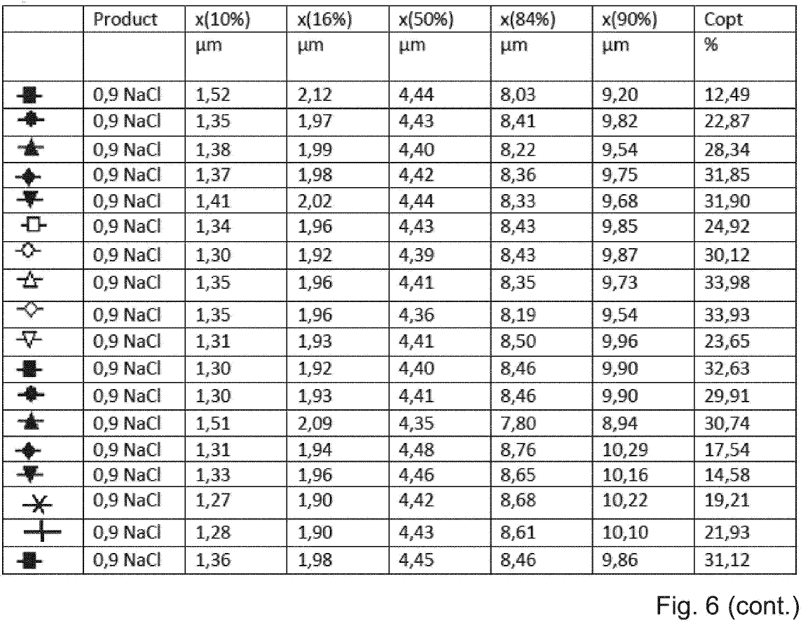

FIG. 6 depicts the nebulization of isotonic saline solution 2.

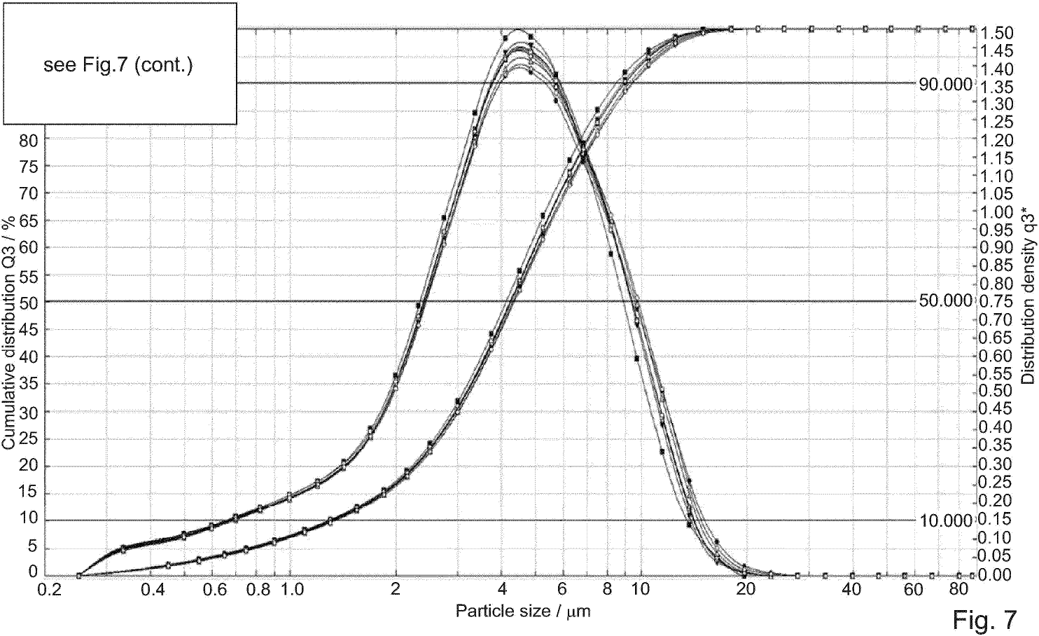

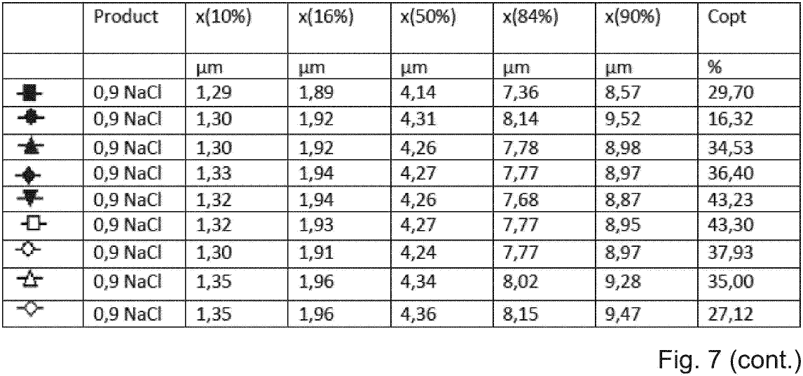

FIG. 7 depicts the nebulization of isotonic saline solution 3.

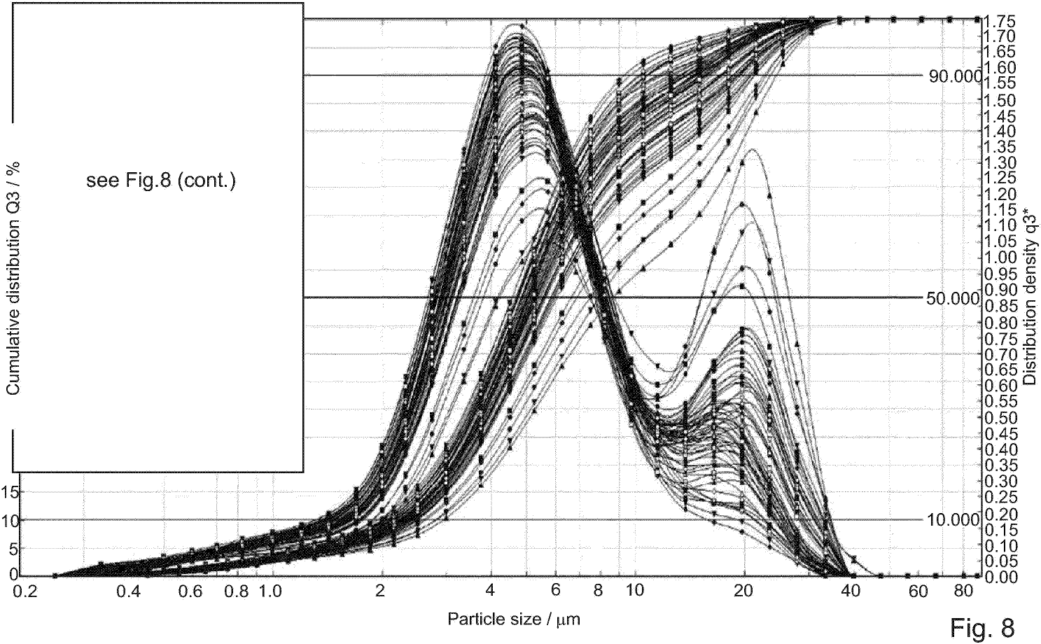

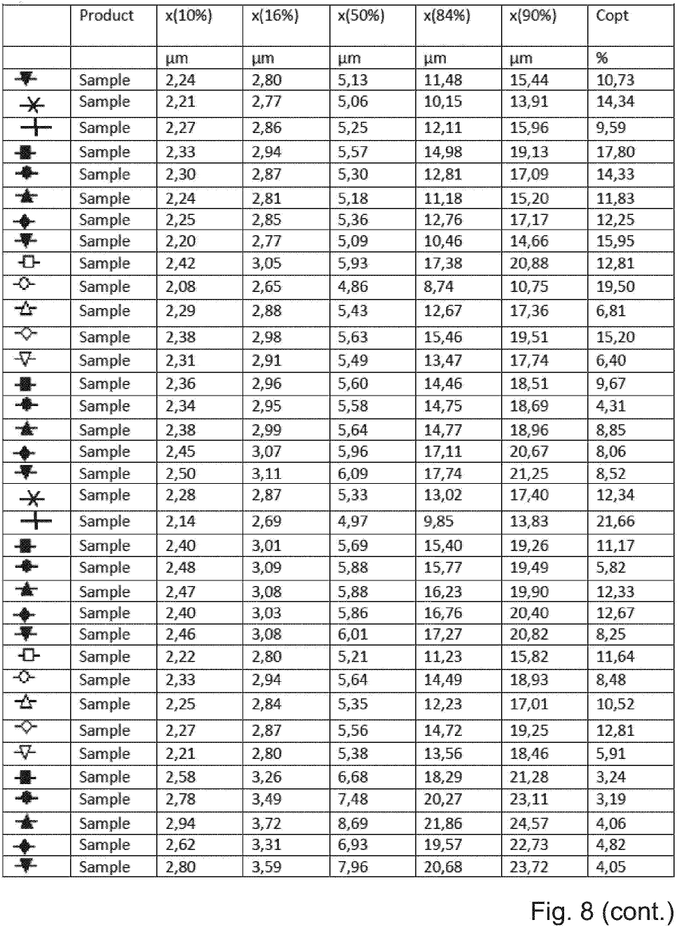

FIG. 8 depicts the nebulization of Example 8 - run 2.

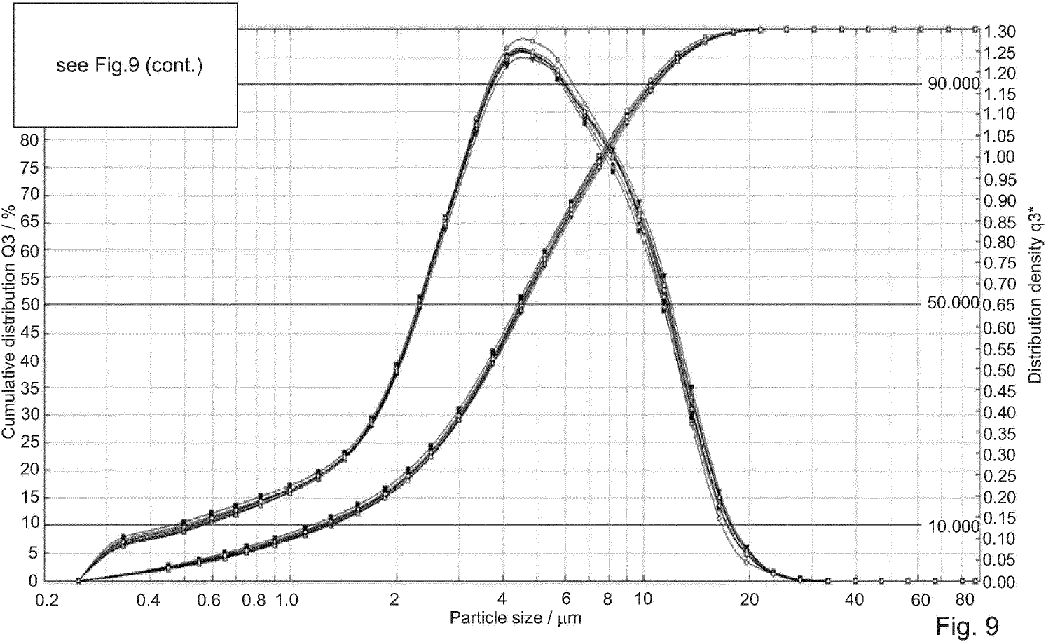

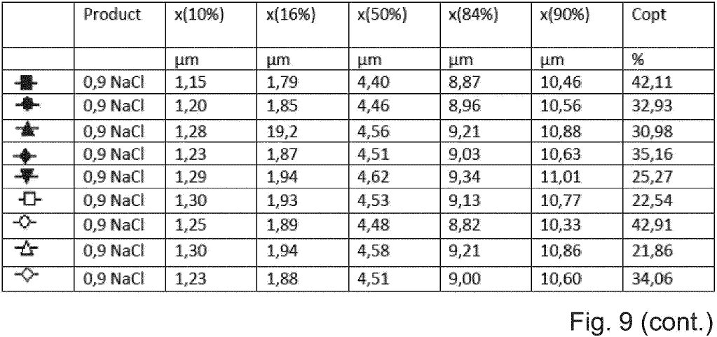

FIG. 9 depicts the nebulization of isotonic saline solution 4.

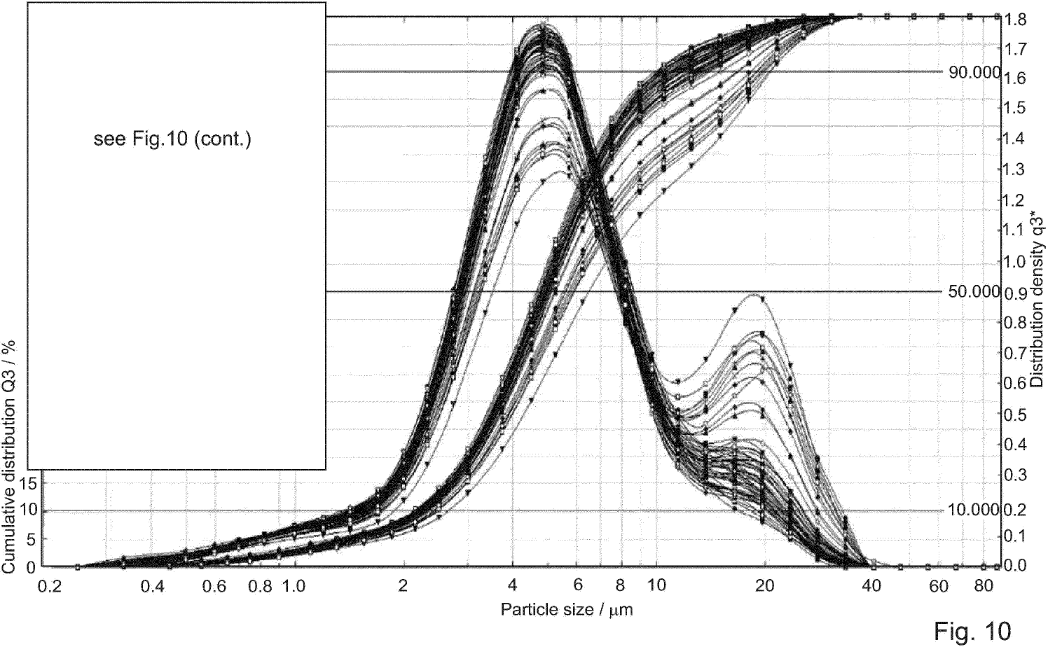

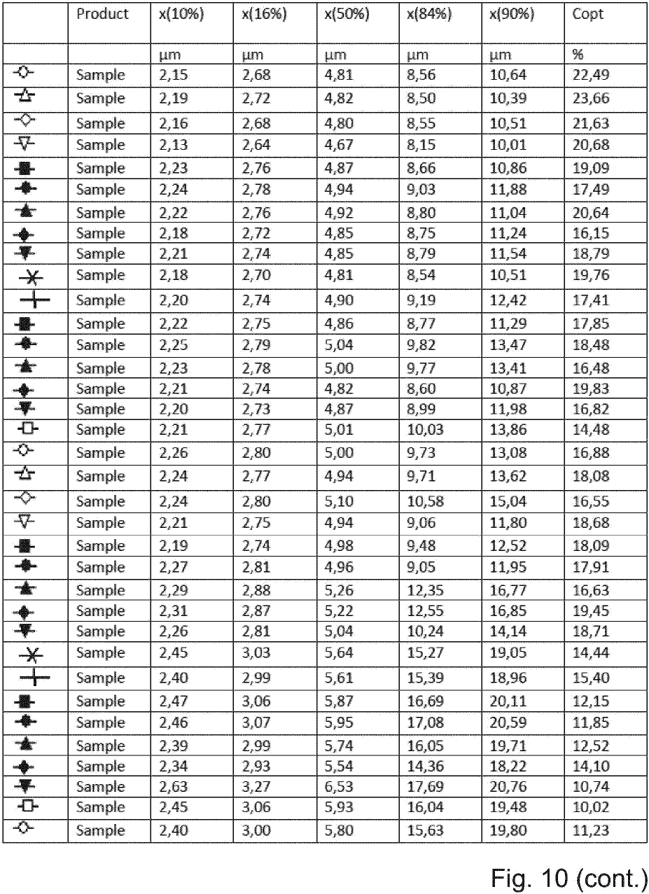

FIG. 10 depicts the nebulization of Example 8 - run 3.

From FIG. 4 it can be seen that the nebulizer, when used with normal saline initially, creates a uniform droplet distribution around a single peak at a VMD of approx. 4 µm, equivalent to a FPF of 74%. The droplet size distribution remains constant throughout the nebulization duration, and is on the order of 0.3 g/min. This droplet size range is well suited for a comparably large fraction of the generated droplets reaching the lung and alveolars.

FIG. 6 shows the nebulization of the solution according to Example 8 with the same nebulizer as used in FIG. 4 directly afterwards. While the droplet distribution is initially similar to that of FIG. 4, over the nebulization duration there is a massive shift of the droplet size towards a second peak at approx. 20 µm, corresponding to a decrease of the initial peak at approx. 4 µm. This corresponds with a severe increase of the VMD to almost twice the diameter, and a reduction of the throughput of solution to 0.12 g/min which is less than the throughput of the previous experiment. These changes in the nebulization performance are detrimental for the clinical application, as the larger droplets are deposited to a larger extent in the mouth or throat and do not reach the target area. In addition, the reduced throughput means that the nebulization of the same dose takes much longer which is inconvenient for the patient and practitioner.

FIGS. 7 and 9 show the nebulization of normal saline nebulized with the same nebulizer directly after the sample described above. For both normal saline samples, an increase in droplet size and a reduction in VMD is apparent compared to the initial normal saline nebulization. The throughput rate is not negatively affected. This shows that the membrane in the nebulizer was impaired in its performance by the nebulization of the previous sample, and the changes could not be reversed by nebulizing two normal saline samples in a row.

FIG. 6 shows a second sample of PEG-ADM formulation according to Example 8. The findings with regard to a second larger peak increasing over the nebulization duration, reduced FPF and reduced throughput are comparable to the first sample shown in FIG. 6.

FIG. 8 shows the nebulization of a normal saline solution, with comparable observations as discussed for FIGS. 7 and 9.

FIG. 10 shows the nebulization of a third sample of PEG-ADM formulation according to Example 8, with comparable findings as discussed in FIG. 6 and FIG. 5.

Overall the nebulization results for Example 8 clearly demonstrate that the formulation leads to changes in the nebulizer performance, resulting in an increased VMD, reduced fine particle fraction and reduced throughput rate. All three parameters are problematic for the therapeutic application through this nebulizer.

### D3 Nebulization of Example 9

Example 9 was nebulized multiple times using the same nebulizer head. Example 9 is a formulation according to the invention. The nebulization experiment with the Example 9 was performed to study if nebulization of the formulation according to Example 9 results in any changes in droplet size or throughput rate during the nebulization, as well as modifications on the performance of the nebulizer for multiple samples nebulized in a row with the identical nebulizer. A new Aerogen® Solo nebulizer was used for the experiment and initially characterized by nebulizing normal saline solution. Afterwards, samples of solutions according to Example 9 were nebulized in total three times, with nebulization of normal saline samples in between to assess changes in the nebulizer caused by the formulation.

Example 9 as well as the isotonic saline solutions were nebulized and analyzed in the order shown in table 6 below. The results are shown in table 6.

GSD means geometric standard deviation. “output rate” in gram per minute (g/min) indicates how many grams of solution are nebulized per minute (throughput). “FPF” in percent (%) means fine particle fraction, which indicates the percentage of particles/droplets below 5 µm. Nebulization properties, expressed as volume median diameter (VDM in micrometer (µm)), of Example 9 and isotonic saline solution.

TABLE 6

| Example | VMD [µm] | GSD | Output rate [g/min] | FPF [%] |
|---|---|---|---|---|
| isotonic saline 1 | 3.57 | 1.7 | 0.17 | 80.6 |
| Example 9 - run 1 | 4.20 | 1.7 | 0.19 | 70.0 |
| isotonic saline 2 | 4.42 | 1.9 | 0.28 | 65.3 |
| Example 9 - run 2 | 4.38 | 1.7 | 0.23 | 67.3 |
| Example 9 - run 3 | 4.78 | 1.8 | 0.30 | 61.6 |
| isotonic saline 3 | 4.33 | 1.8 | 0.32 | 66.6 |

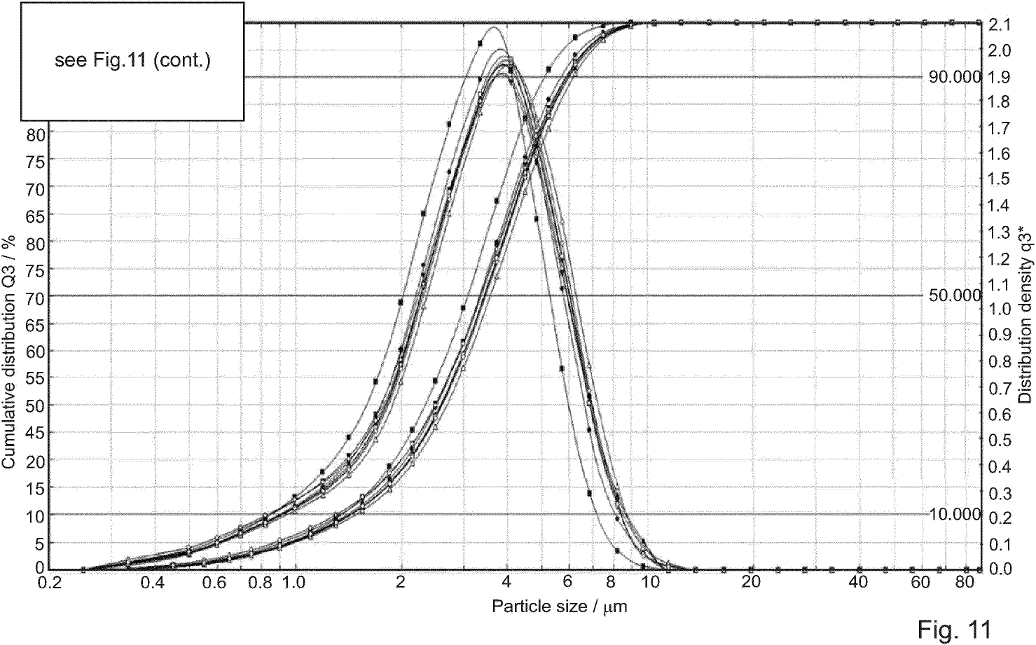

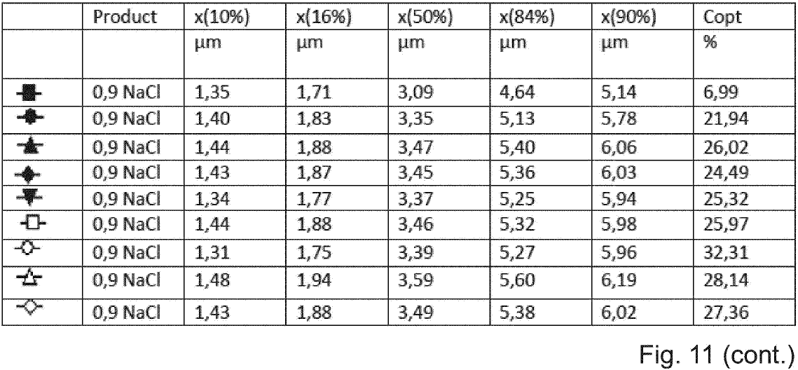

The results are depicted in FIGS. 11 to 16

FIG. 11 depicts the nebulization of isotonic saline solution 1.

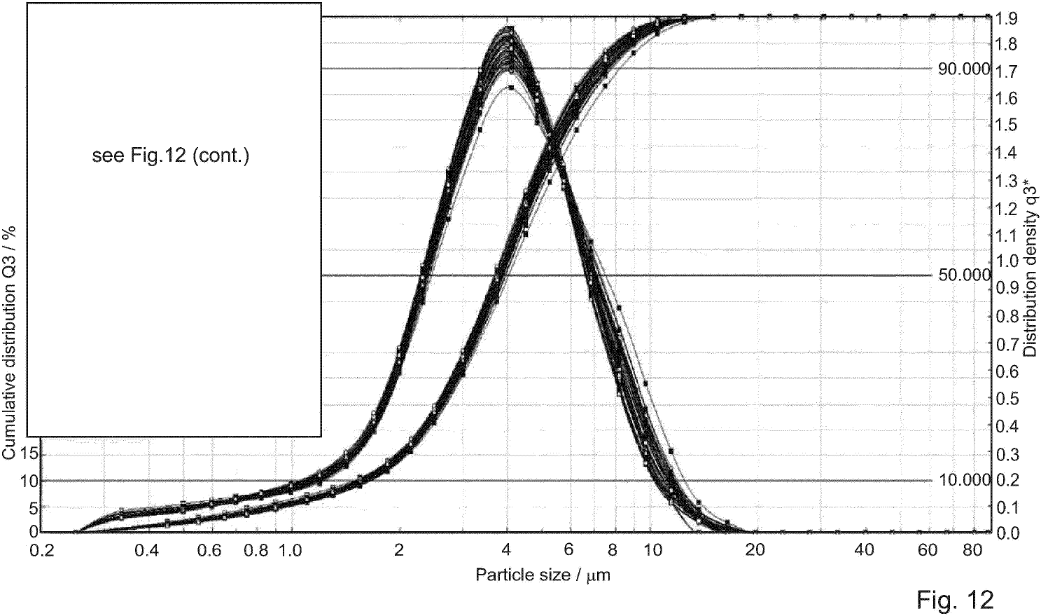

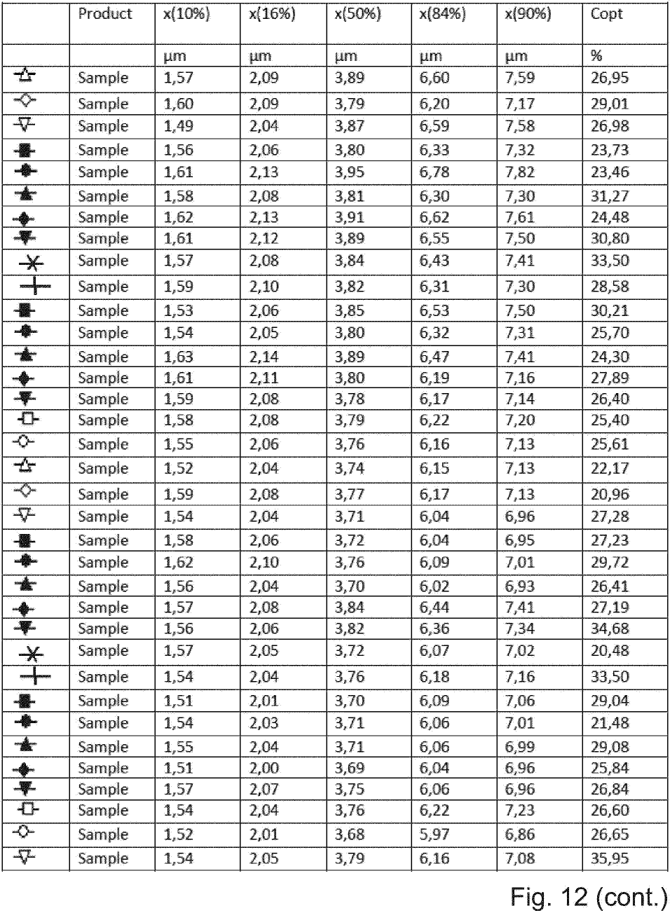

FIG. 12 depicts the nebulization of Example 9 - run 1.

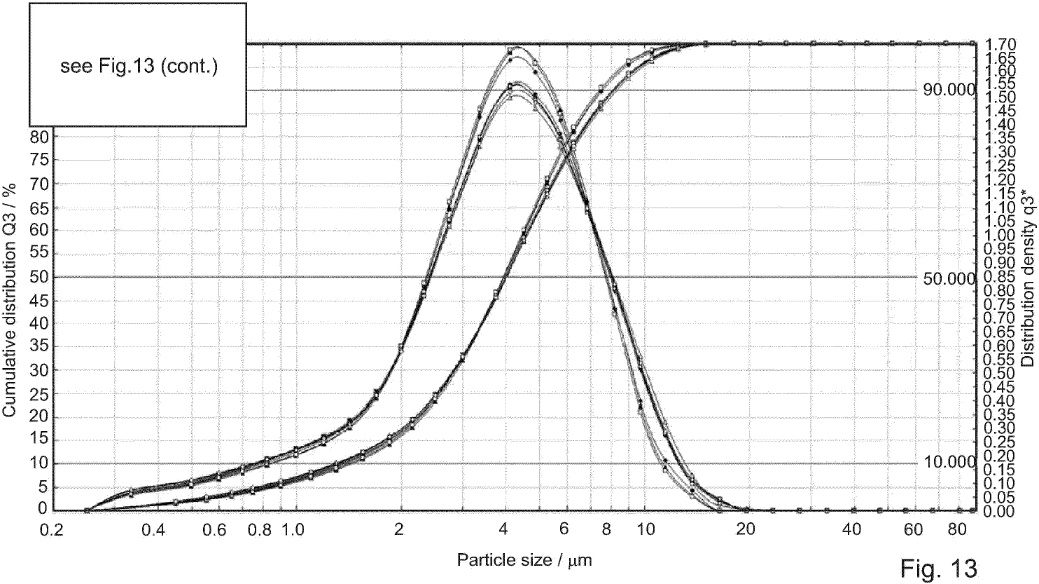

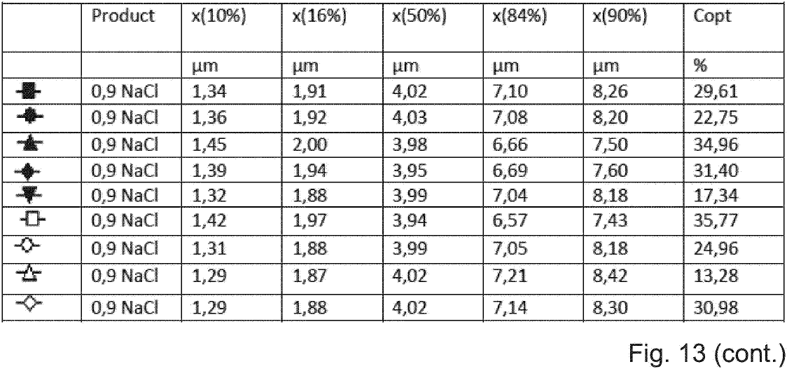

FIG. 13 depicts the nebulization of isotonic saline solution 2.

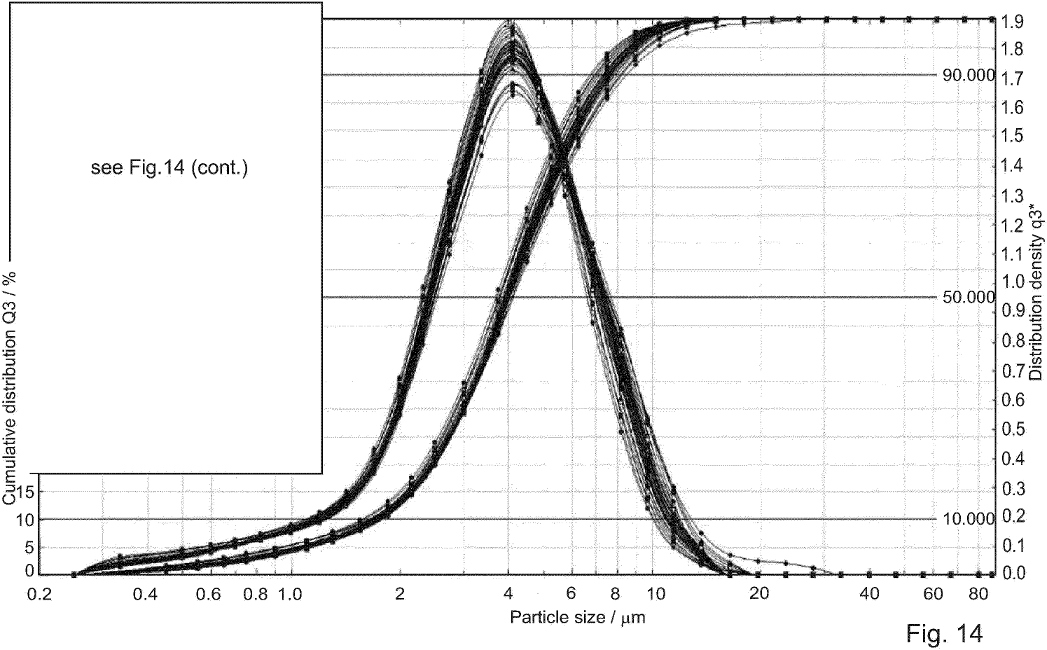

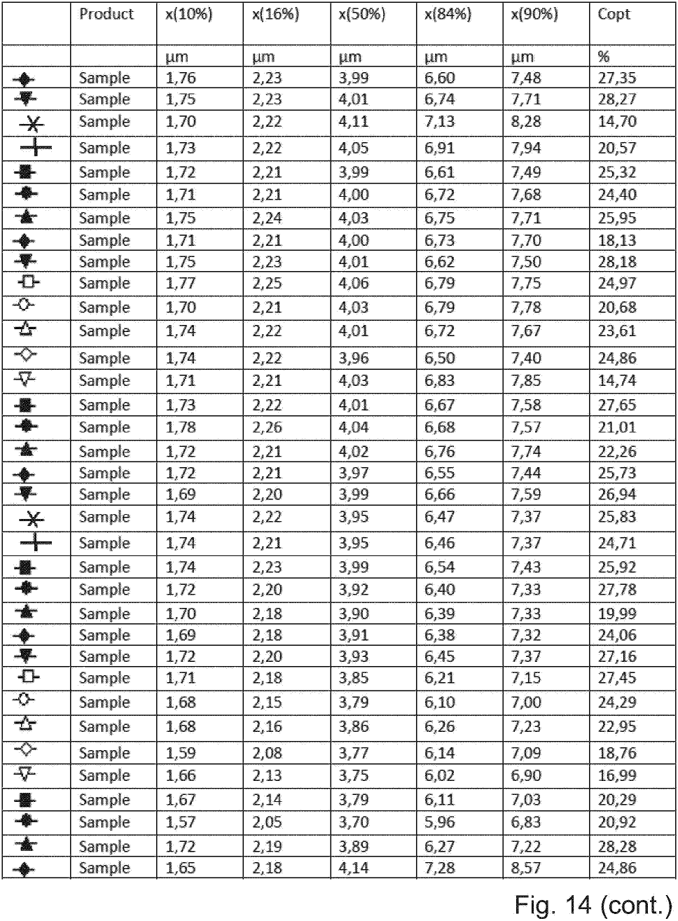

FIG. 14 depicts the nebulization of Example 9 - run 2.

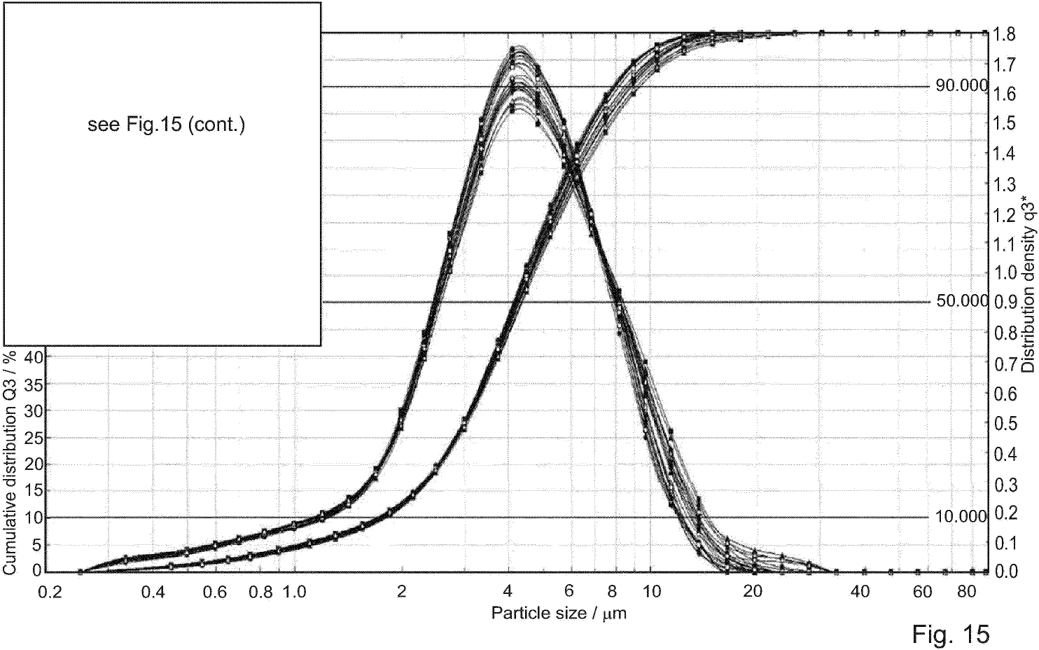

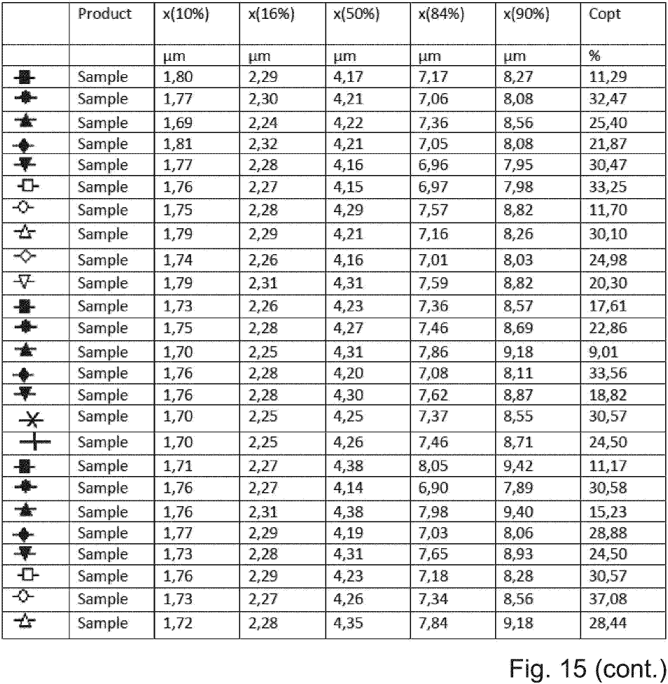

FIG. 15 depicts the nebulization of isotonic saline solution 3.

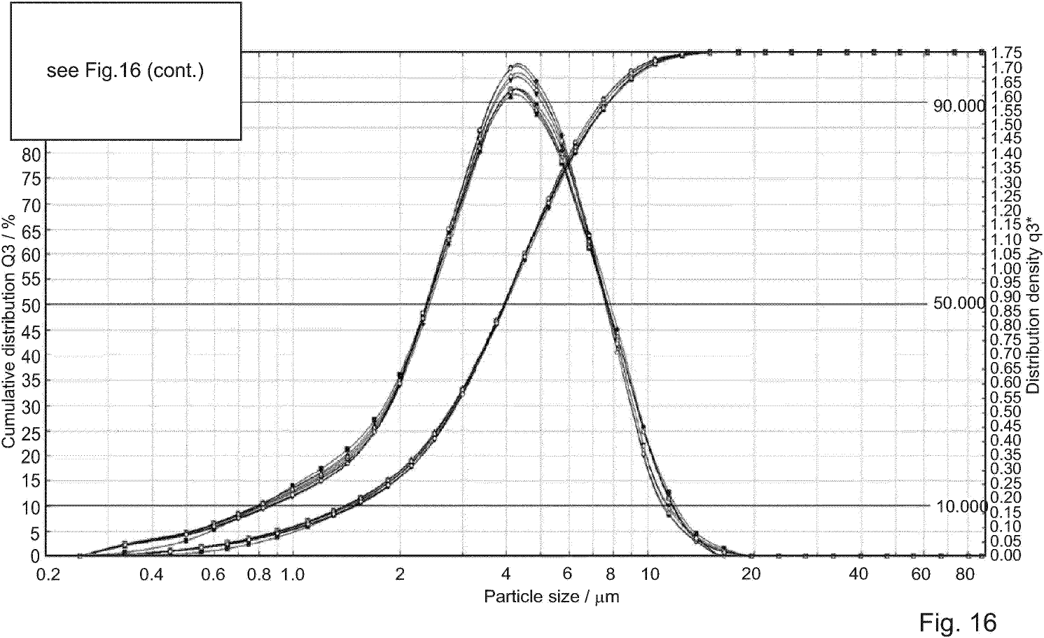

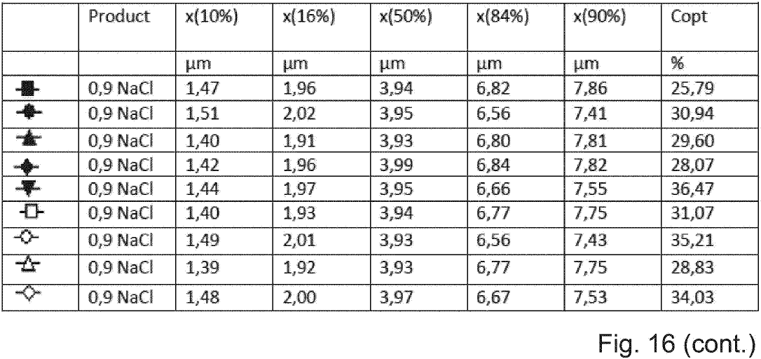

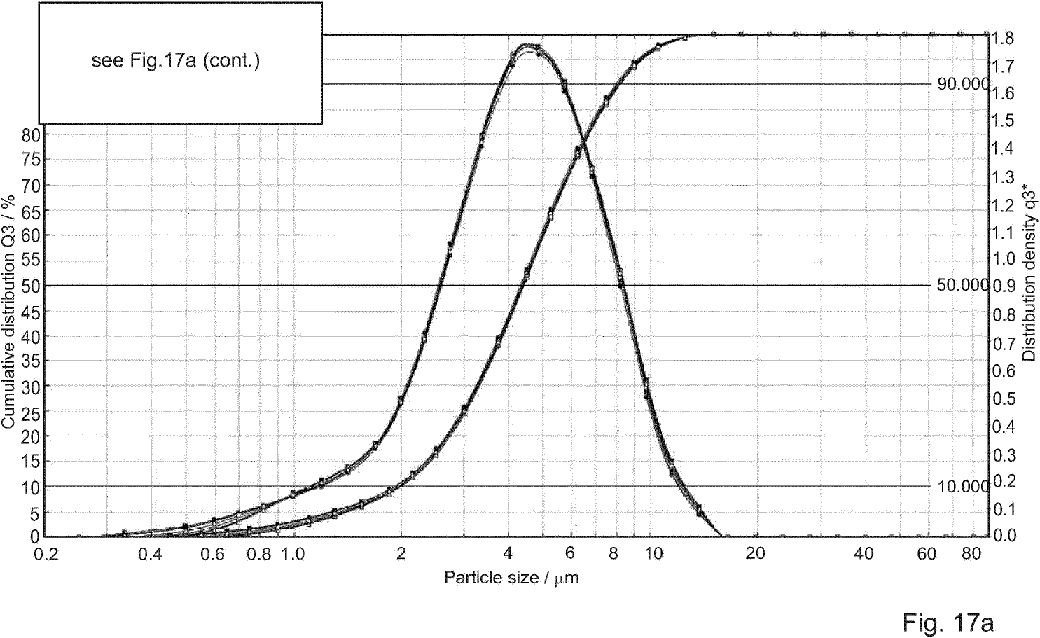

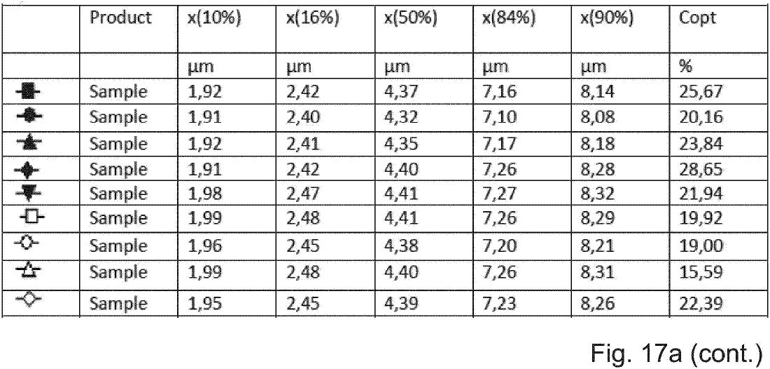

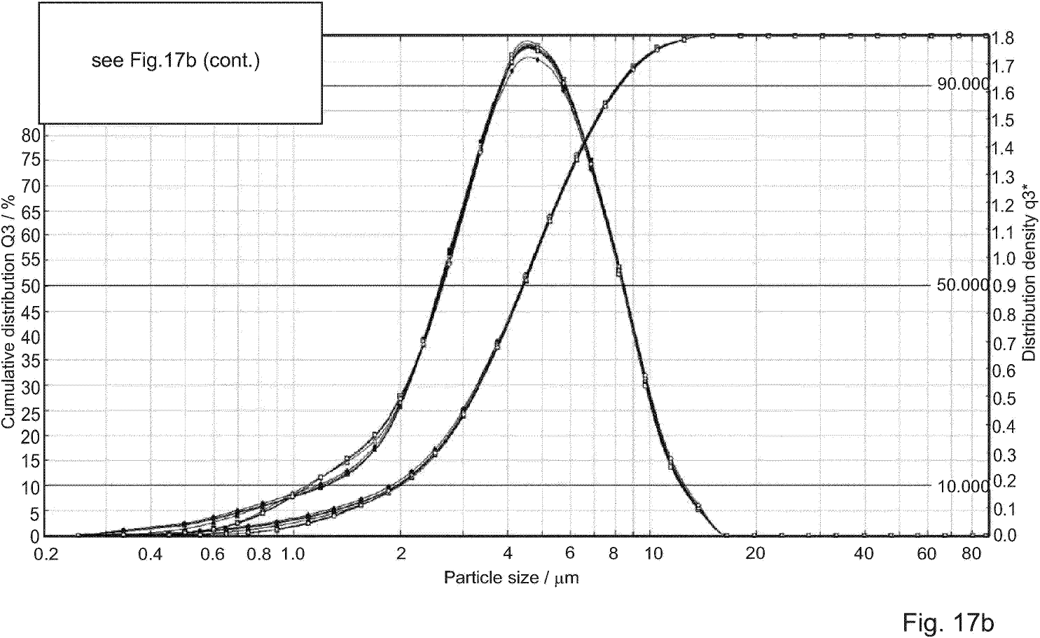

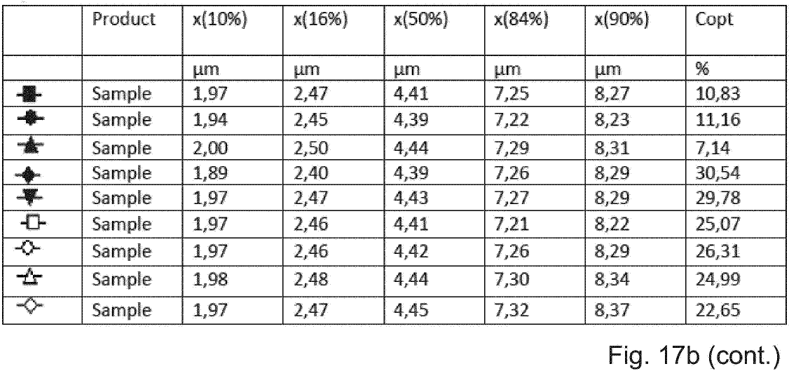

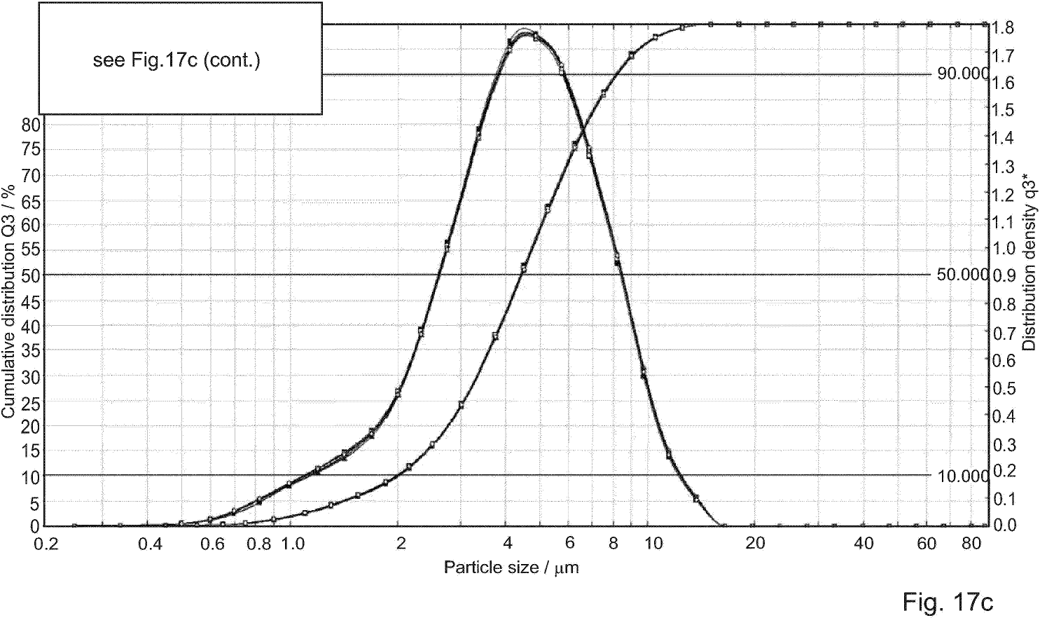

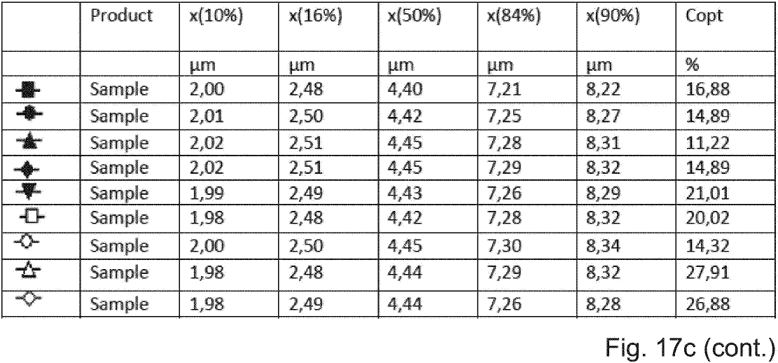

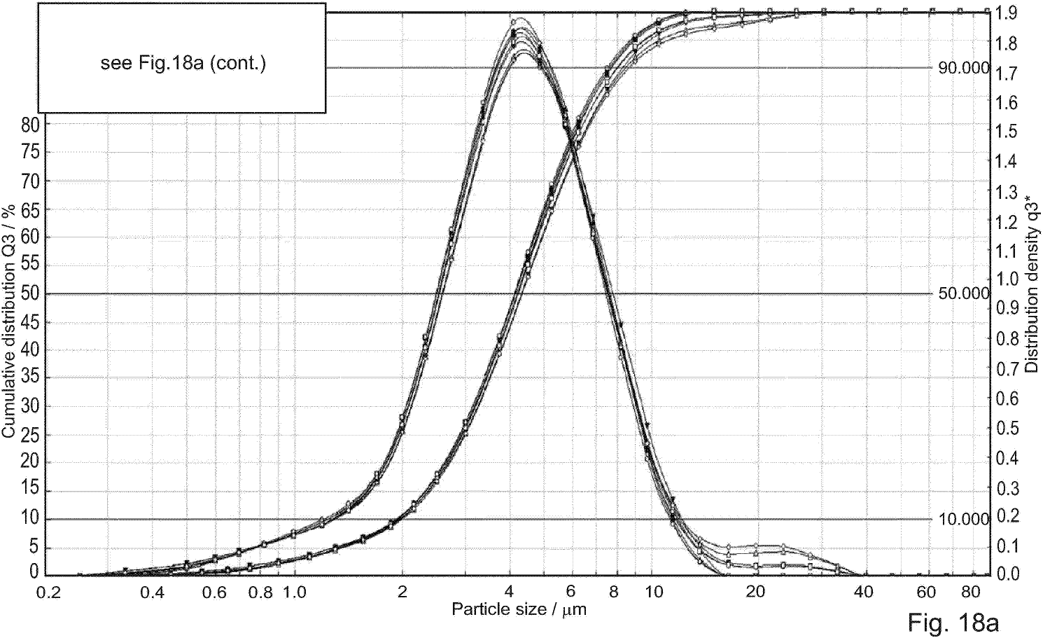

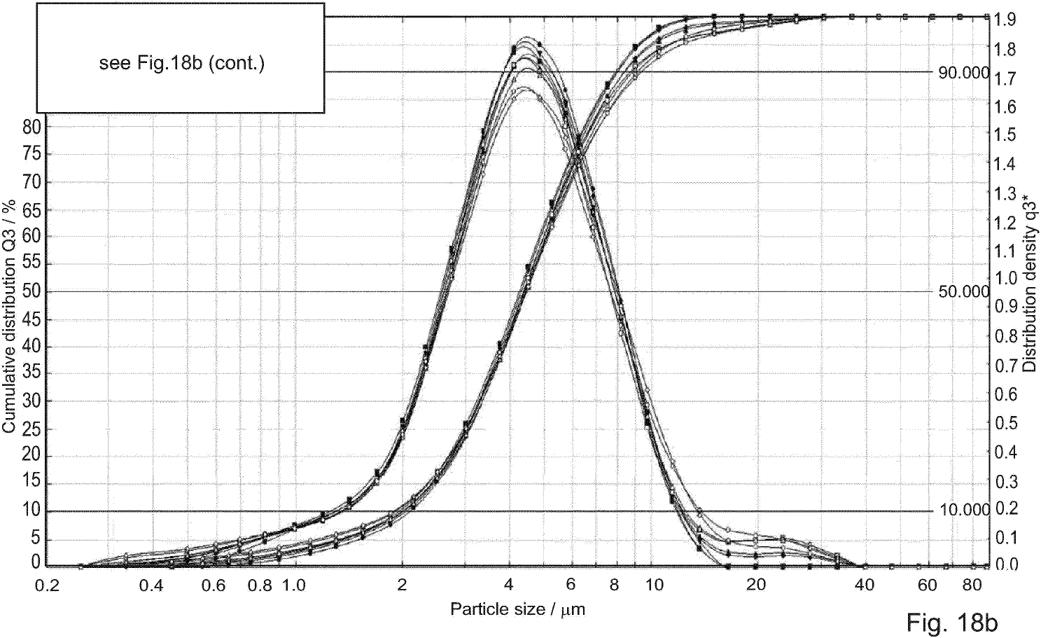

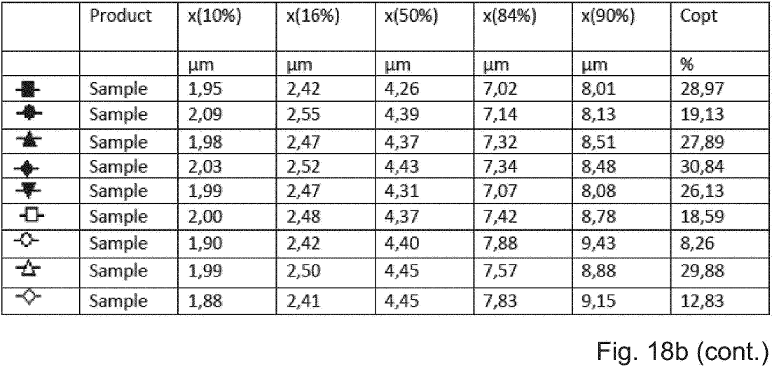

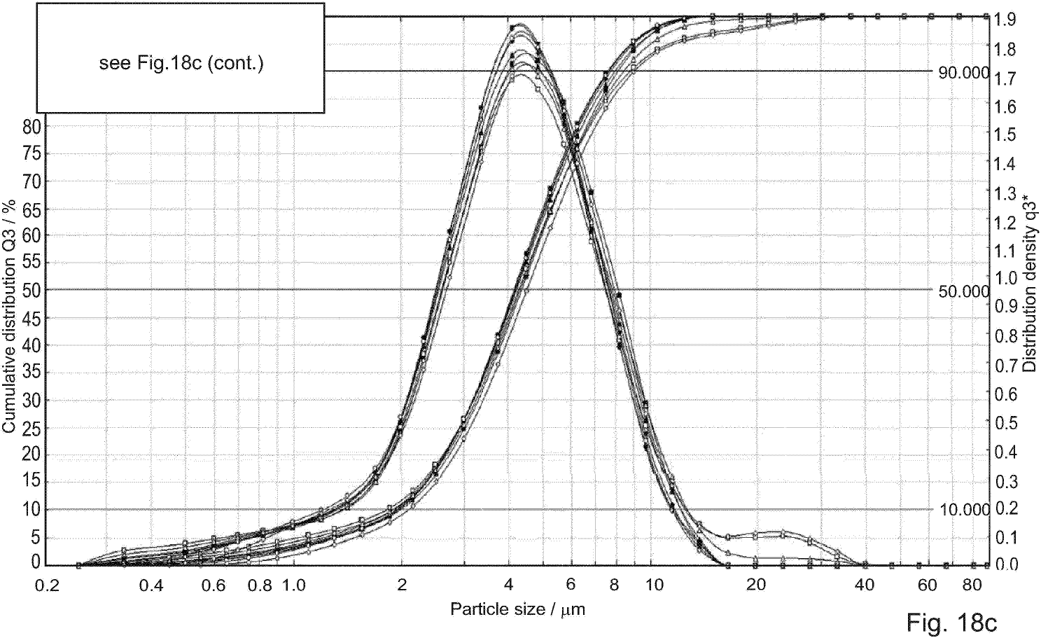

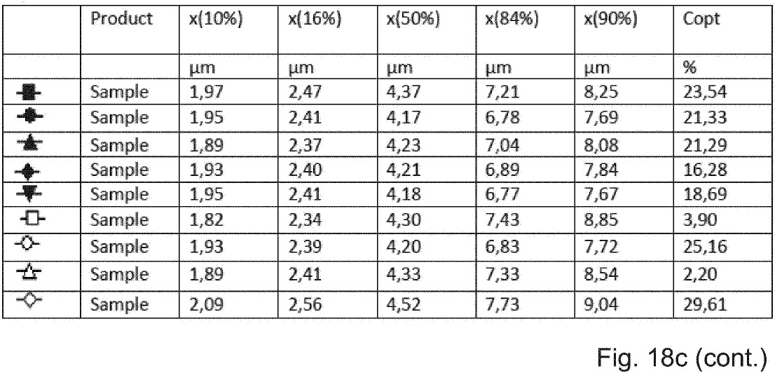

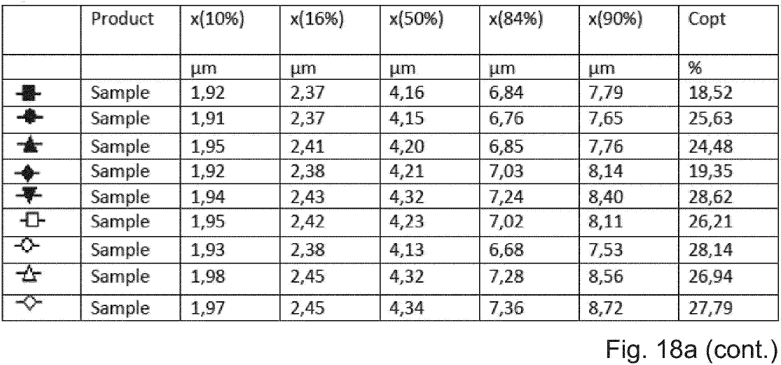

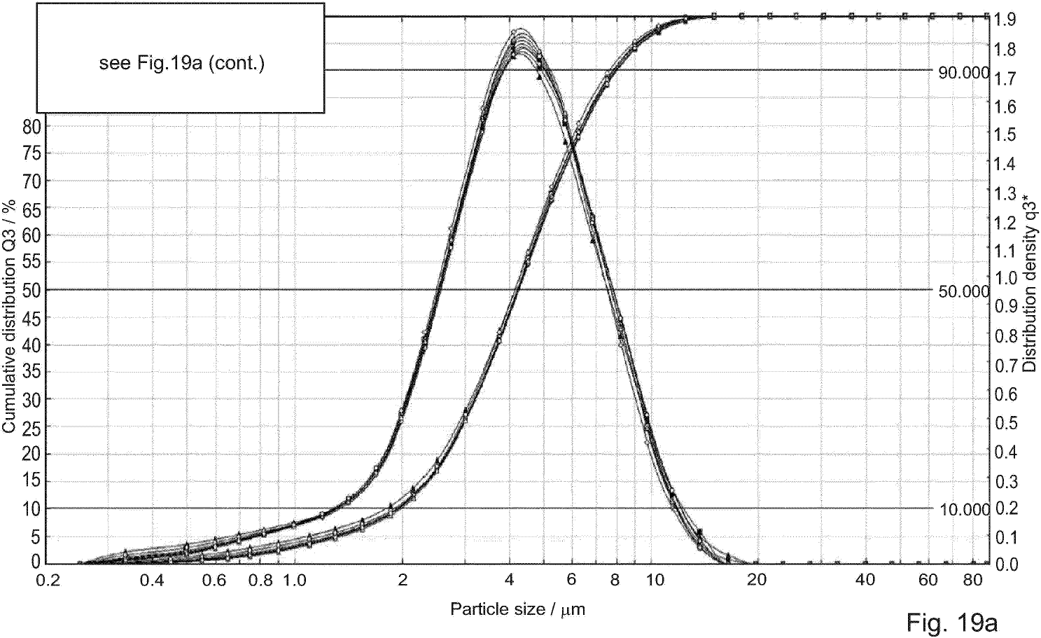

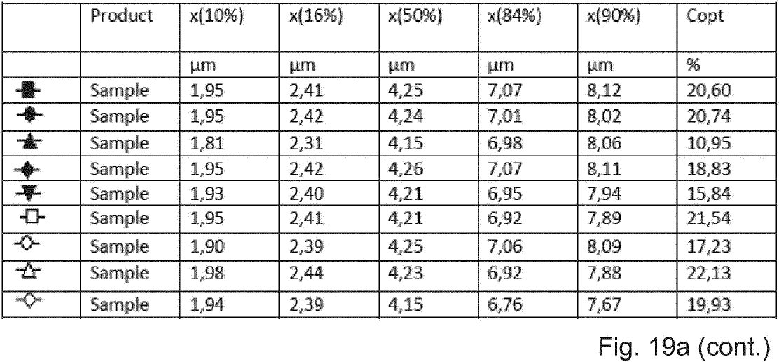

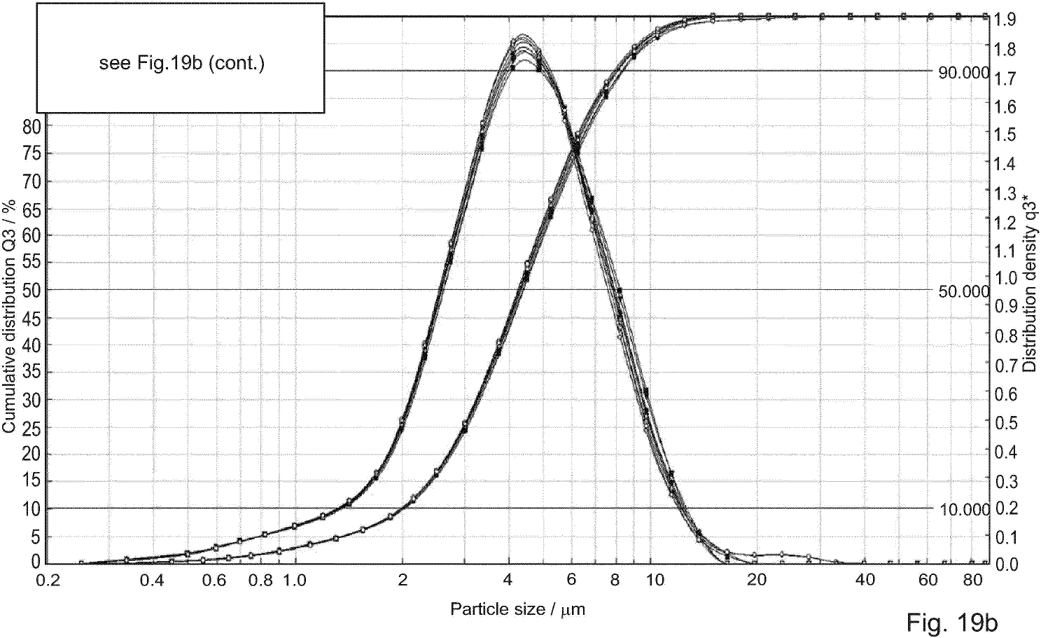

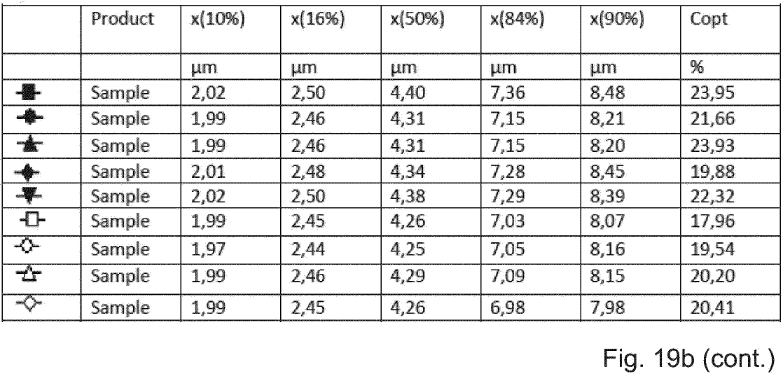

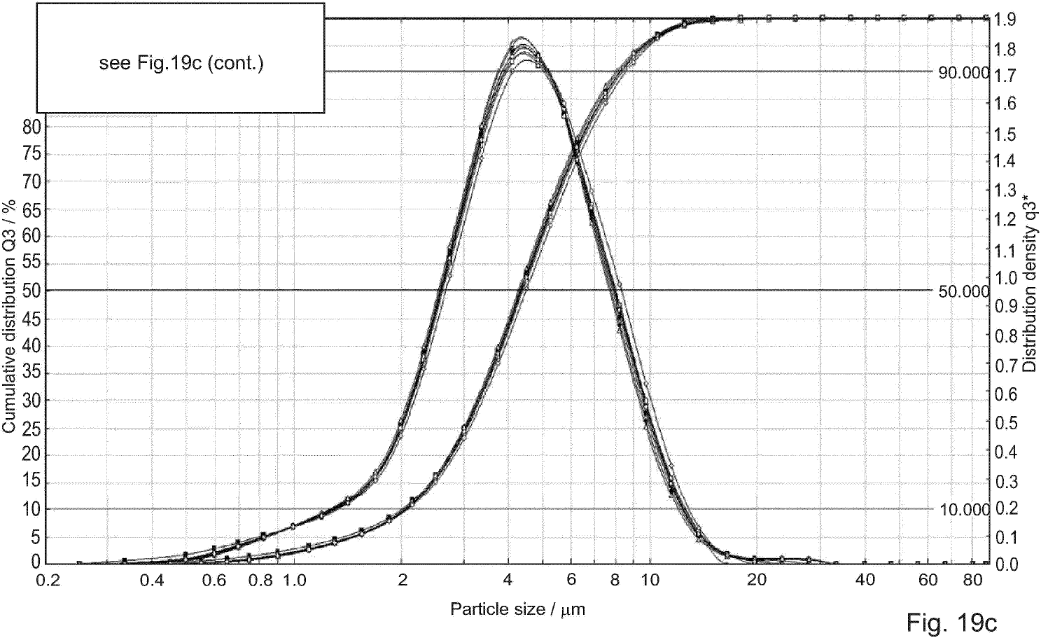

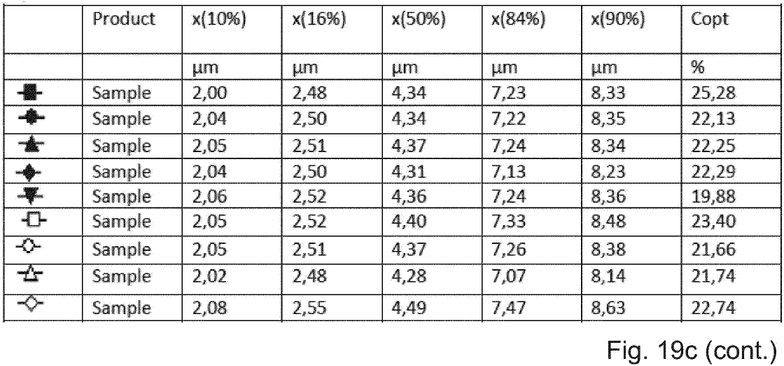

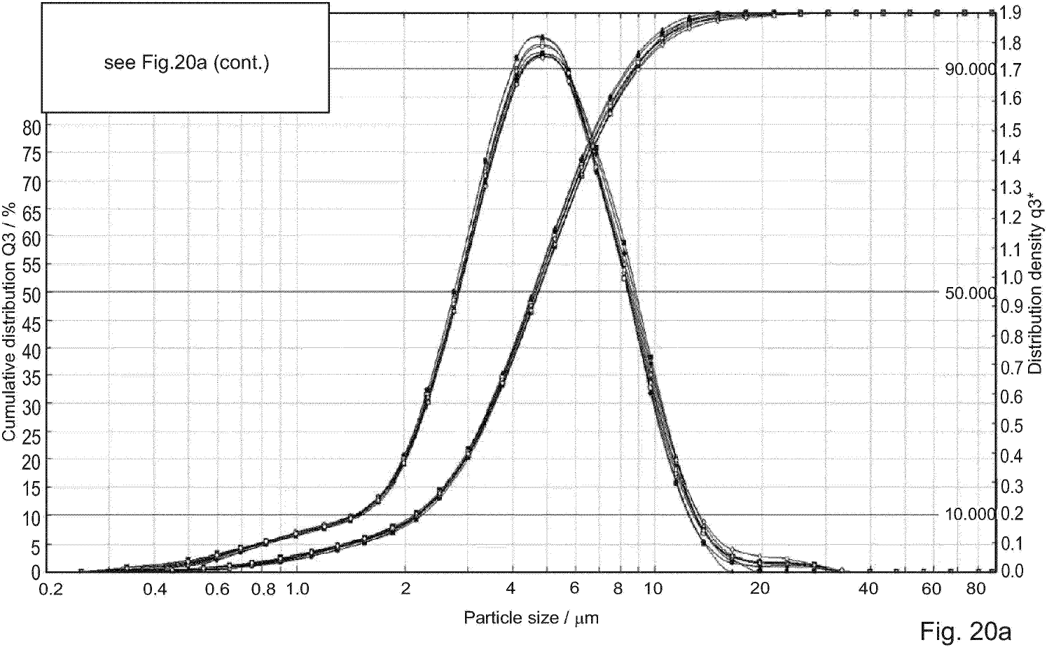

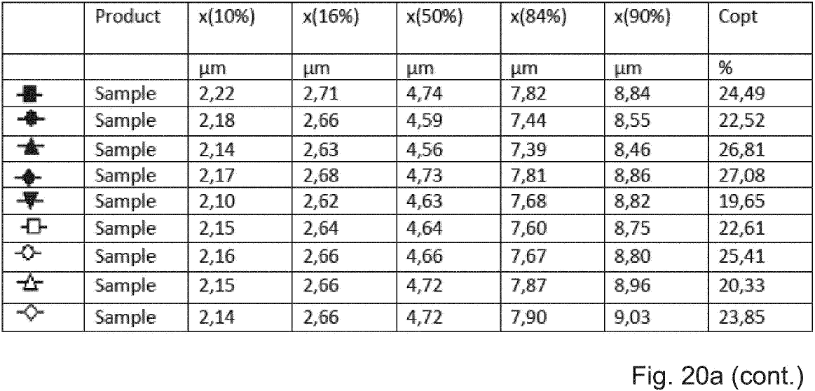

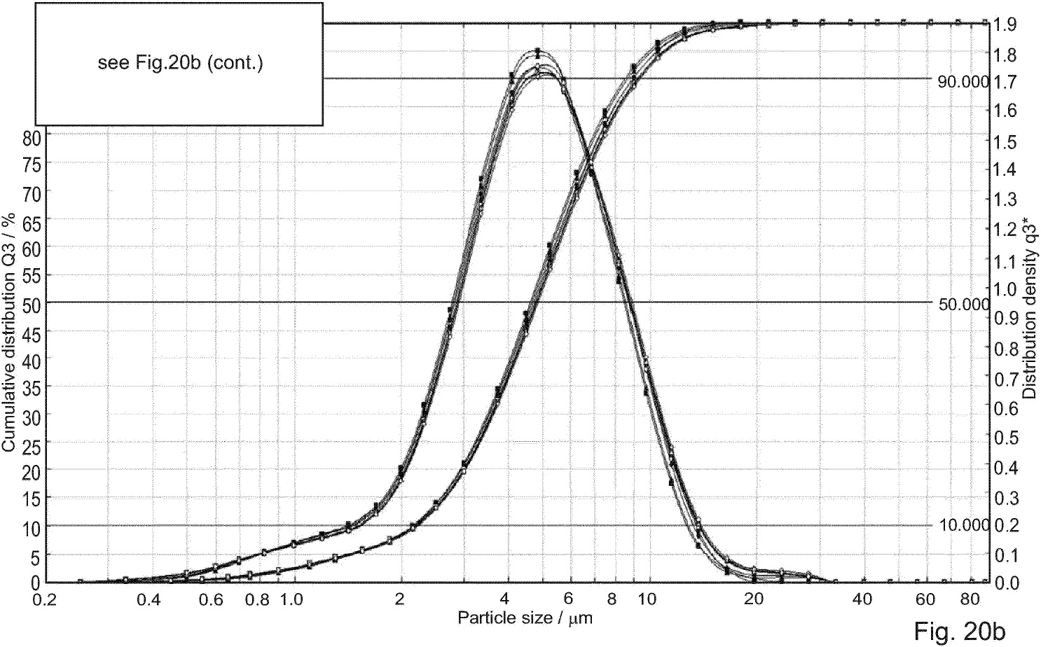

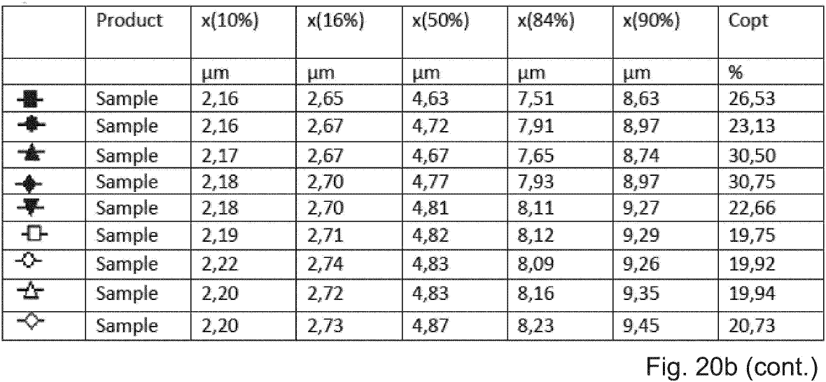

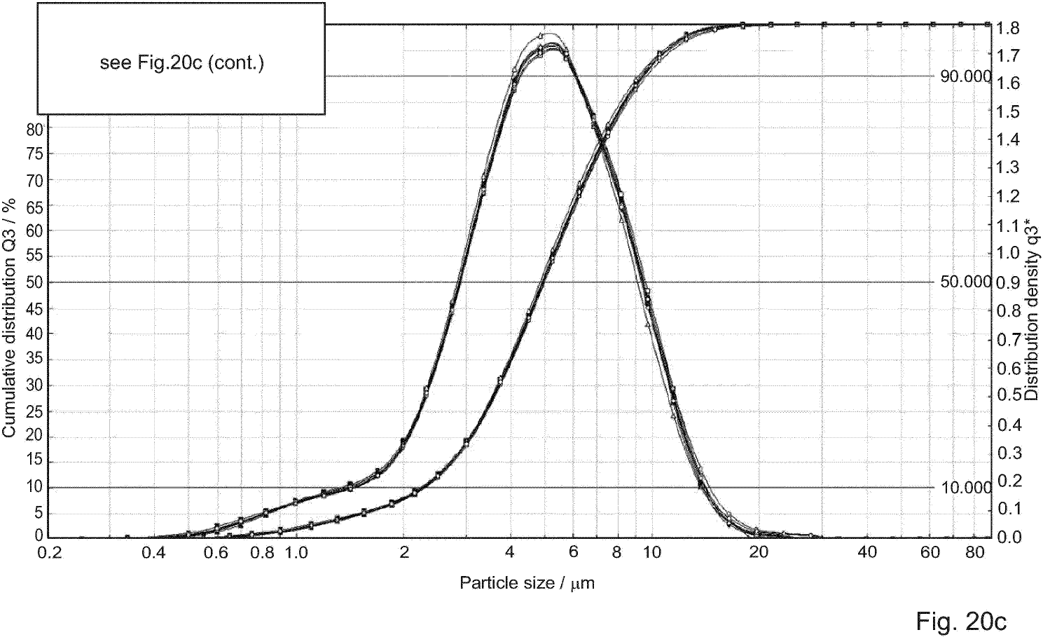

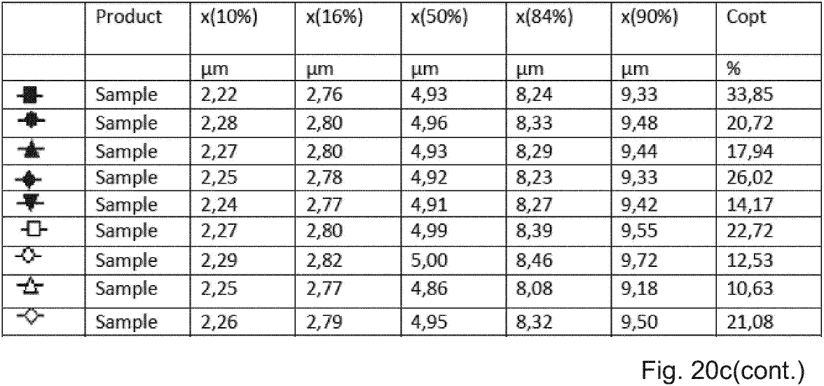

FIG. 16 depicts the nebulization of Example 9 - run 3.

From FIG. 11 it can be seen that the nebulizer when used with normal saline initially creates a uniform droplet distribution around a single peak at a VMD of approx. 3.6 µm, equivalent to a FPF of 81%. The droplet size distribution remains constant throughout the nebulization duration, and is on the order of 0.2 g/min. This droplet size range is well suited for a comparably large fraction of the generated droplets reaching the lung and alveolars.

FIG. 13 shows the nebulization of the solution according to Example 9 with the same nebulizer as used in FIG. 11 directly afterwards. The droplet distribution is slightly higher than that of FIG. 11, but remains almost constant throughout the nebulization without formation of a second peak at larger droplet sizes. The VMD is consistently at 4.2 µm, and the FPF remains at 70%. Also the throughput is not reduced compared to the throughput of the normal saline sample described above. The droplet size remains at a level which is beneficial for the therapeutic application of delivering to the lower lungs. The fact that a relatively moderate decrease in concentration leads to this major difference of changes to the nebulizer performance is unexpected.

FIG. 15 shows a sample of normal saline nebulized with the same nebulizer directly after the sample shown in FIG. 13. The VMD and FPF remain comparable to the previous sample, and the throughput rate slightly increases. There are no significant trends over the nebulization duration with regard to changes in droplet size or widening of the distribution.

FIG. 11 and FIG. 14 show two consecutive nebulization of formulation samples according to Example 9 with the same nebulizer as used in the experiments described in FIG. 15. Even during the second and third nebulization with the moderately diluted PEG-ADM formulation, no trends in the VMD and FPF are apparent compared to the first nebulization described in FIG. 13. The same applies to the throughput rate which is very comparable or even higher.

FIG. 16 shows an additional sample of normal saline solution which is nebulized after completing the two nebulizations shown in FIG. 11 and FIG. 14. Again, no detrimental changes in droplet size distribution are observable compared to the previous nebulizations.

Overall, it was clearly shown that the formulation according to Example 9 can be nebulized three times in a row with the same nebulizer head with a relative continuous droplet size as well as output rate and without detrimental effects on VMD, FPF or throughput rate.

### D4 Summary of Nebulization Results -Comparison of Results for Examples 8 and 9 as Well As Placebo:

The results presented above demonstrate that the nebulization of reconstituted solutions according to Example 8 lead to severe changes in the nebulization characteristics for nebulization using the Aerogen® Solo. These changes only occurred for the samples containing PEG-ADM and not for placebo samples containing trehalose and various other excipients. Surprisingly, the effects of PEG-ADM on the nebulizer performance are concentration-dependent and are almost completely absent for the more dilute formulation according to Example 9.

When further comparing Example 8 with Example 9 it can be seen, that Example 9 is superior than Example 8:

Example 9 shows a continuous droplet size after multiple nebulization. This is beneficial as the same nebulizer can be used several times.

Example 9 shows a smaller droplet size. This is beneficial as a higher fraction of these droplets can reach the lower lungs.

Example 9 shows a higher output rate of although the droplet size is smaller than Example 8 even for several nebulizations fo the PEG-ADM solution in a row. The skilled would expect that a smaller droplet size would result in a smaller output rate. Surprisingly, Example 9 shows smaller droplets, the output is significantly higher. This allows more formulation (PEG-ADM) to be nebulized, which is available to the patient when inhaling.

In summary, formulation according to Example 9 shows superior characteristics for clinical application and delivery of PEG-ADM to the lower lungs than the formulation according to Example 8.

### D5 Nebulization of Reconstituted Examples 10 - 15

For Examples 10 to 15, the respective lyphilizate was reconstituted in 3.3 mL water thereby giving the concentrations listed in table 7 below:

TABLE 7

Composition of reconstituted Examples 10 to 15

| COMPOSITI ON | RECONSTITUTED EXAMPLES | | | | | |
|---|---|---|---|---|---|---|
| | 10 | 11 | 12 | 13 | 14 | 15 |
| Drug substance | mg/mL | mg/mL | mg/mL | mg/mL | mg/mL | mg/mL |
| PEG-ADM [concentration ADM] | 0.4 [0.052] | 7 [0.909] | 2.464 [0.32] | 2.464 [0.32] | 2.464 [0.32] | 2.464 [0.32] |
| Excipients | | | | | | |
| Citric acid anhydrous | 3.59 | 3.59 | 3.59 | 3.59 | 3.59 | 3.59 |
| Sodium hydroxide | 1.494 | 1.494 | 1.494 | 1.494 | 1.494 | 1.494 |
| Sodium chloride | 1.19 | 1.19 | 1.19 | 1.19 | 1.19 | 1.19 |
| Trehalose dihydrate | 33.33 | 33.33 | 13.1 | 20.33 | 49.33 | 70.66 |

These formulations were nebulized and measured. Reconstituted Examples 10 - 15 were nebulized three times each using one nebulizer head per Example. The nebulization experiments with the reconstituted Examples 10 - 15 were performed to study if nebulization of the formulations result in any changes in droplet size or throughput rate during the nebulization, as well as modifications on the performance of the nebulizer for multiple samples nebulized in a row with the identical nebulizer. A new Aerogen® Solo nebulizer was used for each Example, and initially characterized by nebulizing normal saline solution. The nebulization results for reconstituted Examples 10 - 15 are shown in table 8-1 to table 8-6 below.

TABLE 8-1

Nebulization results for reconstituted Example 10

| Reconstituted Example | VMD [µm] | GSD | Output rate [g/min] | FPF [%] |
|---|---|---|---|---|
| isotonic saline 1 | 4.52 | | 0.35 | |
| Example 10 - run 1 | 4.79 | 1.7 | 0.36 | 59.9 |
| Example 10 - run 2 | 4.83 | 1.7 | 0.37 | 59.2 |
| Example 10 - run 3 | 4.85 | 1.7 | 0.37 | 59.0 |

TABLE 8-2

Nebulization results for reconstituted Example 11

| Reconstituted Example | VMD [µm] | GSD | Output rate [g/min] | FPF [%] |
|---|---|---|---|---|
| isotonic saline 1 | 4.61 | | 0.37 | |
| Example 11 - run 1 | 5.38 | 1.6 | 0.17 | 54.1 |
| Example 11 - run 2 | 6.05 | 1.7 | 0.16 | 51.9 |
| Example 11 - run 3 | 5.50 | 1.7 | 0.13 | 53.8 |

TABLE 8-3

Nebulization results for reconstituted Example 12

| Reconstituted Example | VMD [µm] | GSD | Output rate [g/min] | FPF [%] |
|---|---|---|---|---|
| isotonic saline 1 | 4.39 | | 0.30 | |
| Example 12 - run 1 | 4.79 | 1.7 | 0.27 | 62.9 |
| Example 12 - run 2 | 5.05 | 1.7 | 0.31 | 59.8 |
| Example 12 - run 3 | 4.82 | 1.7 | 0.30 | 62.0 |

TABLE 8-4

Nebulization results for reconstituted Example 13

| Reconstituted Example | VMD [µm] | GSD | Output rate [g/min] | FPF [%] |
|---|---|---|---|---|
| isotonic saline 1 | 4.41 | | 0.33 | |
| Example 13 - run 1 | 5.03 | 1.8 | 0.30 | 57.7 |
| Example 13 - run 2 | 5.54 | 1.8 | 0.32 | 52.9 |
| Example 13 - run 3 | 5.51 | 1.8 | 0.31 | 52.9 |

TABLE 8-5

Nebulization results for reconstituted Example 14

| Reconstituted Example | VMD [µm] | GSD | Output rate [g/min] | FPF [%] |
|---|---|---|---|---|
| isotonic saline 1 | 4.47 | | 0.36 | |
| Example 14 - run 1 | 4.63 | 1.7 | 0.23 | 63.1 |
| Example 14 - run 2 | 4.79 | 1.7 | 0.24 | 61.3 |
| Example 14 - run 3 | 4.87 | 1.7 | 0.24 | 60.4 |

TABLE 8-6

Nebulization results for reconstituted Example 15

| Reconstituted Example | VMD [µm] | GSD | Output rate [g/min] | FPF [%] |
|---|---|---|---|---|
| isotonic saline 1 | 4.5 | | 0.33 | |
| Example 15 - run 1 | 5.19 | 1.7 | 0.28 | 55.3 |
| Example 15 - run 2 | 5.34 | 1.7 | 0.29 | 53.6 |
| Example 15 - run 3 | 5.49 | 1.7 | 0.31 | 50.9 |

Overall, nebulization of the reconstituted Examples 10 to 15 using the Aerogen Solo nebulizer was successful and resulted in generation of droplets. For the reconstituted Example 10, nebulization was very fast with a high consistency of droplet size and fine particle fraction.

For the reconstituted Example 11, the throughput was reduced due to the higher content of PEG-ADM, which also affected the droplet size and the fine particle fraction.

For the reconstituted Examples 12 to 15, the results regarding throughput and droplet size are relatively comparable, and relatively little change is apparent over the three nebulizations.

In summary, the lyophilizates according to Examples 10 to 15 could be reconstituted. For the reconstituted Examples 10 to 15, it was shown that these could be nebulized. All Examples 10 to 15 show a fine particle fraction (FPF, [%]) above 50 %.

1. A pharmaceutical formulation comprising:
PEG-ADM, wherein the PEG-ADM is a compound according to formula (I), 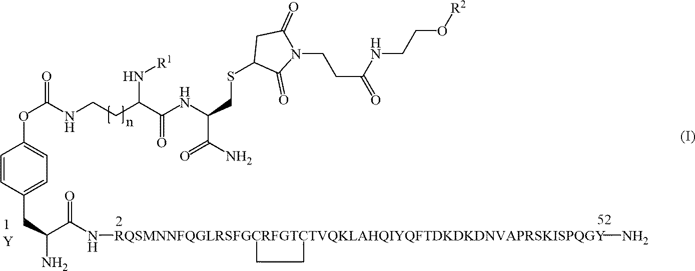
in which
n represents the number 0, 1, 2 or 3,
R¹ represents hydrogen, methyl, ethyl, n-propyl or isopropyl,
R² represents linear or branched PEG 20 kDa to 80 kDa endcapped with a methoxy-group,
And/or a hydrate thereof, solvate thereof, salt thereof, pharmaceutically acceptable salt thereof, and/or a solvate of a salt thereof;
a pH regulator; and
trehalose and/or a hydrate thereof, solvate thereof, salt thereof, pharmaceutically acceptable salt thereof, and/or a solvate of a salt thereof.

2. The pharmaceutical formulation according to claim 1, wherein the pharmaceutical formulation comprises 60 wt.-% to 98 wt.-% trehalose, wherein concentrations of components thereof are based on total weight of the pharmaceutical formulation.

3. The pharmaceutical formulation according to claim 1, wherein the pharmaceutical formulation comprises
1 wt.-% to 15 wt.% of PEG-ADM and/or a hydrate thereof, solvate thereof, salt thereof, pharmaceutically acceptable salt thereof, and/or a solvate of a salt thereof;
0.01 wt.-% to 25 wt.% of the pH regulator; and
60 wt.-% to 98 wt.-% trehalose and/or a hydrate thereof, solvate thereof, salt thereof, pharmaceutically acceptable salt thereof, and/or a solvate of a salt thereof;
wherein concentrations of components thereof are based on total weight of the pharmaceutical formulation.

4. The pharmaceutical formulation according to claim 1 wherein the PEG-ADM is the compound according to formula (Ia) 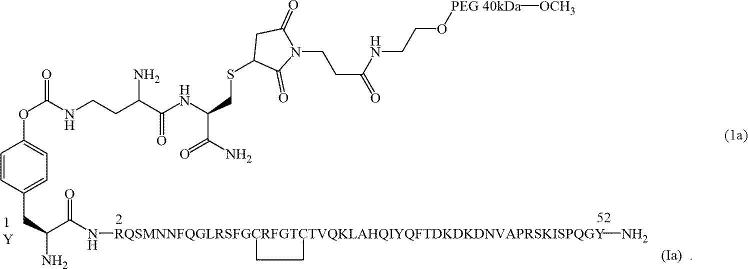
.

5. The pharmaceutical formulation according to claim 1, wherein the pharmaceutical formulation is a lyophilizate.

6. A liquid pharmaceutical formulation comprising:
a 0.04 mg/mL to 145 mg/mL of PEG-ADM, wherein the PEG-ADM is a compound according to formula (I), 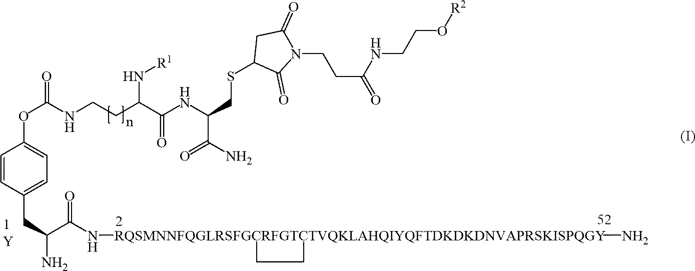
in which
n represents the number 0, 1, 2 or 3,
R¹ represents hydrogen, methyl, ethyl, n-propyl or isopropyl,
R² represents linear or branched PEG 20 kDa to 80 kDa endcapped with a methoxy-group,
And/or a hydrate thereof, solvate thereof, salt thereof, pharmaceutically acceptable salt thereof, and/or a solvate of a salt thereof;
b. a solvent;
c. a pH regulator;
d. an osmolarity regulator; and
e. trehalose;
wherein the presence of the osmolarity regulator (component d) is optional;
wherein the pharmaceutical formulation has a pH between 3 and 5; and
wherein concentrations of components thereof are based on the total volume of the liquid pharmaceutical formulation.

7. The pharmaceutical formulation according to claim 6, wherein the liquid pharmaceutical formulation comprises
0.385 mg/mL to 77 mg/mL PEG-ADM, wherein the PEG-ADM is a compound according to formula (I) or formula (Ia), and/or a hydrate thereof, solvate thereof, salt thereof, pharmaceutically acceptable salt thereof, and/or a solvate of a salt thereof; and
10 mg/mL to 100 mg/mL trehalose.

8. The pharmaceutical formulation according to claim 6, wherein the liquid pharmaceutical formulation comprises a lyophilizate and a solvent.

9. The pharmaceutical formulation according to claim 1 for inhalation.

10. A medicament comprising the pharmaceutical formulation according to claim 1 or medicament comprising the pharmaceutical formulation in combination with an inert nontoxic pharmaceutically suitable excipient, optionally in combination with a further active ingredient.

11. A combined pharmaceutical dose form comprising components (1) and (2), wherein
component (1) comprises a pharmaceutical formulation according to claim 1 and
component (2) comprises a solvent.

12. A combination pack comprising component (1) and (2), wherein
component (1) comprises the pharmaceutical formulation of claim 1, a medicament thereof comprising, or a combined pharmaceutical dose form comprising said formulation ; and component (2) comprises a nebulizer, optionally a mesh nebulizer.

13. The pharmaceutical formulation according to claim 1, a medicament thereof a combined pharmaceutical dose form comprising said formulation or a combination pack comprising said formulation for use in treatment and/or prevention of one or more diseases.

14. The pharmaceutical formulation, the medicament, the combined pharmaceutical dose form, or the combination pack according to claim 13 for use in treatment and/or prevention of one or more diseases, wherein the disease is selected from
pulmonary disorders, optionally pulmonary hypertension; secondary pulmonary hypertension; pulmonary hypertension following pulmonary embolism with and without acute cor pulmonale; primary pulmonary hypertension; chronic obstructive pulmonary disease; asthma; acute pulmonary edema; chronic pulmonary edema; allergic alveolitis; pneumonitis due to inhaled organic dust; pneumonitis due to inhaled particles of fungal, actinomycetic or other origin; acute chemical bronchitis; acute chemical pulmonary edema and/or chronic chemical pulmonary edema (optionally after inhalation of phosgene, nitrogen oxide); neurogenic pulmonary edema; acute pulmonary manifestations due to radiation; chronic pulmonary manifestations due to radiation; acute and/or chronic interstitial lung disorders optionally but not restricted to drug-induced interstitial lung disorders, optionally secondary to Bleomycin treatment); acute lung injury (ALI); acute lung injury (ALI) in adult or child including newborn; acute respiratory distress syndrome (ARDS); acute respiratory distress syndrome (ARDS) in adult or child including newborn; ALI/ARDS secondary to pneumonia and sepsis, aspiration pneumonia and ALI/ARDS secondary to aspiration optionally but not restricted to aspiration pneumonia due to regurgitated gastric content; ALI/ARDS secondary to smoke gas inhalation; transfusion-related acute lung injury (TRALI), ALI/ARDS or acute pulmonary insufficiency following surgery; trauma or burns, ventilator induced lung injury (VILI); lung injury following meconium aspiration; pulmonary fibrosis; and mountain sickness;
ALI/ARDS secondary to pneumonia caused by bacterial infection of the lungs, optionally, but not restricted to, bacterial pneumonia caused by Pneumococci, Haemophilus Influenzae, Mycoplasma Pneumoniae, Chlamydia species, Enterococci, beta-hemolytic Streptococci, Staphylococci, Gram-negative Enterobacteriaceae, Pseudomonas species, Klebsiella species, Acinetobacter species, Legionella species, and Mycobacteria;
ALI/ARDS secondary to pneumonia caused by viral infections optionally, but not restricted to, Influenza viruses (optionally caused by strains of serotypes H1N1, H5N1, H7N9), Corona viruses (optionally SARS-CoV, the pathogen of severe acute respiratory syndrome (SARS), MERS-CoV, the pathogen of Middle East respiratory syndrome (MERS), and SARS-CoV-2 the pathogen of COVID-19 pandemic), Respiratory-Syncytial-Virus (RSV), and Cytomegalovirus (CMV);
ALI/ARDS secondary to pneumonia caused by fungal infections optionally, but not restricted to, fungal pneumonia caused by Pneumocystis Jirovecii;
ALI/ARDS secondary to pneumonia irrespective of the context of pneumonia origin as optionally for community acquired pneumonia (CAP) as well as for hospital acquired pneumonia (HAP), optionally for HAP acquired in the context of artificial ventilation (VAP);
ALI/ARDS secondary to pneumonia irrespective of the diverse pathoanatomical appearances of pneumonias optionally, but not restricted to, lobar (optionally affecting an entire lung lobe), lobular (optionally affecting smaller lung lobules), interstitial (optionally diffuse affection of the lung tissue);
ALI/ARDS secondary to pneumonia occurring in consequence of bacterial and/or virus infection;
ALI/ARDS secondary to pneumonia occurring in consequence of a bacterial superinfection of a primary lung affection by viruses; and
prevention and/or treatment of lung dysfunction after lung transplantations.

15. The lyophilizate according to claim 5 obtainable by freeze-drying of a liquid pharmaceutical formulation.

16. The liquid pharmaceutical formulation according to claim 6 obtainable by mixing a lyophilizate with a solvent.

17. A method for preparation of the pharmaceutical formulation according to claim 5, comprising :
step 1. Providing at least components a, c and e; and
step 2. Mixing the components provided in step 1;
step 3: freeze-drying the pharmaceutical formulation obtained after any one of 1 and/or 2
whereby the pharmaceutical formulation 5 is obtained.
